(19) United States
(12) Patent Application Publication
(10) Pub. No.: US 2009/0217396 A1
(43) Pub. Date: Aug. 27, 2009

(54) Inflammation models in neurodegenerative and arthritic disorders

(76) Inventors: Stephanos Kyrkaniders, Rochester, NY (US); M. Kerry O'Banion, Pittsford, NY (US)

Correspondence Address: Ballard Spahr Andrews & Ingersoll, LLP, SUITE 1000, 999 PEACHTREE STREET, ATLANTA, GA, 30309-3915, US

(21) Appl. No.: 11/667,392

(22) PCT Filed: Nov. 14, 2005
(86) PCT No.: PCT/US05/42058

§ 371 Date: Mar. 4, 2008

Related U.S. Application Data
(60) Provisional application No. 60/627,604, filed on Nov. 12, 2004.
(60) Provisional application No. 60/646,097, filed on Jan. 20, 2005.

(51) Int. Cl. A01K 67/027; C12N 15/11; C12N 15/00; C12N 15/87; C12N 5/06

(52) U.S. Cl. 800/13; 536/23.5; 536/23.2; 435/320.1; 435/325; 435/455

(57) ABSTRACT

Disclosed are compositions and methods for inducing temporally conditional mediators or inflammation and the transgenic animals produced by these compositions and method that can be used as models of inflammatory disease.

### CROSS-REFERENCE TO RELATED APPLICATIONS

This application claims benefit of U.S. Provisional Application No. 60/627,604, filed Nov. 12, 2004 and of U.S. Provisional Application No. 60/646,097 filed Jan. 20, 2005, which are hereby incorporated herein by reference in their entirety.

### STATEMENT REGARDING FEDERALLY SPONSORED RESEARCH

This invention was made with government support under Grants RO1 NS33553 and R21 NS048522 awarded by the National Institutes of Health. The government has certain rights in the invention.

### BACKGROUND

There are a number of diseases and disorders related to inflammation, as well as a number of pathways and molecules relate to inflammation. Models for studying these diseases and disorders are helpful in identifying and testing potential pharmaceuticals to treat these diseases and disorders. Disclosed are animal models which conditionally express one or more inflammatory molecules either spatially or temporally, as well as the nucleic acids to construct these models. Also disclosed are methods of using the models.

### SUMMARY

Disclosed are methods and compositions related to vectors, cells, transgenic animals, and methods of making and using thereof related to inflammation molecules.

### BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows IL-1β^{XAT} an excisionally activated transgene. IL-1β^{XAT} is a bicistronic gene comprised of the cytomegalovirus promoter (CMV), followed by a “floxed” transcriptional termination cassette [symbol] STOP [symbol] the human IL-1 RA peptide secretion signal (ss) fused to the mature human IL-1β ORF (ssIL-1β), the reporter lacZ gene and the bovine growth hormone poly A mRNA tail (pA). An internal ribosomal entry signal facilitates translation and expression of the second ORF, lacZ, at approximately 45% of the first ORF.

FIG. 2 shows that Cre-mediated activation of the inducible IL-1β^{XAT} transgene. The IL-1β^{XAT} gene was transfected into the murine fibroblast NIH 3T3 cell line. Transient expression of Cre recombinase following co-transfection of the expression vector pRc/CMV-CreWT resulted in IL-1β^{XAT} activation and higher levels of IL-1β mRNA detected by RT-PCR, as well as lacZ expression assessed by X-gal histochemistry (10×). Control conditions included (a) plain NIH 3T3 cells, as well as (a) cells co-transfected with IL-1β^{XAT} and (c) the pRc/CMV-backbone vector, which displayed background levels of IL-1β and lacZ expression presumably due to minimal spontaneous read-through from the strong CMV promoter.

FIG. 3 shows that CrePr induces loxP-directed IL-1β^{XAT} excisional recombination and gene activation. The IL-1β^{XAT} gene was transiently transfected into 293HGLVP/CrePr cells [Maguire-Zeiss K A, et al. (2002). Neurobiol Aging 23:977-84] and the expression of IL-1β and lacZ was evaluated following RU486 (10-7M) administration. (A) Activation of Cre recombinase by RU486 resulted in up-regulation of both IL-1β and lacZ mRNA as assessed by RT-PCR. For demonstration purposes, an IL-1β standard curve (1 ug-10-5 ug) is included in this panel. (B) Concomitantly, significantly higher levels of secreted IL-1β protein were found in the supernatant media of RU486-treated cells as assessed by ELISA for human IL-1β. (C) The expression of the reporter gene β-galactosidase was also confirmed by Xgal histochemistry: naïve cells present only minimal levels of background staining, whereas addition of RU486 in the culture media resulted in significant increase in the number of X-gal positive cells. (D) IL-1β^{XAT} excisional DNA recombination was confirmed by PCR of genomic DNA extracts from cells treated with plain growth media as well as media containing RU486 (10-7M) using a primer set (UP & LP) that flanked the [symbol] STOP [symbol] sequence. PCR amplification of cells under plain media yielded a full-length product (˜3 Kb), indicative of a dormant IL-1β^{XAT} state. In contrast, RU486-treated cells yielded a PCR product of 1 Kb in size, indicative of DNA recombination and excision of the [symbol] STOP [symbol] cassette.

FIG. 4 shows that IL-1β^{XAT} activation results in expression of biologically potent IL-1β cytokine. The biological potency of the transgene-derived IL-1β cytokine was evaluated in vitro as follows. Murine fibroblasts were treated with conditioned media collected from cultured NIH 3T3 cells that had been previously transfected with Cre-induced IL-1β^{XAT} as described in FIG. 2 above (co-transfection with the pRc/CMV-creWT vector). COX-2 transcript levels was measured in the target cells (murine fibroblasts) and was employed as a measure of IL-1β biological potency. Conditioned media were incubated with the neutralizing antibodies for 2 hours at 37° C. prior to addition to target cells. (A) Conditioned medium collected form naïve NIH 3T3 cells (containing <3.9 pg/mL hIL-1β as determined by ELISA) were placed on murine fibroblasts, which in turn showed low levels of murine COX-2 mRNA. Moreover, (B) conditioned medium from NIH 3T3 cells transfected with IL-1β^{XAT}+pRc/CMV-backbone vector (contained <3.9 pg/mL hIL-1β) also showed low levels of murine COX-2 mRNA. In contrast, (C) conditioned medium from IL-1β^{XAT}+pRc/CMV-CreWT transfected NIH 3T3 cells (1 ng/mL hIL-1β) significantly induced COX-2 mRNA in the target cells; (D) pre-incubation of the conditioned medium with a control rabbit IgG antibody (5 μg/mL IgG1 isotype) had minimal effects on COX-2 regulation. However, (E) pre-incubation of the conditioned medium with a rabbit anti-hIL-1β (5 μg/mL IgG1) antibody attenuated the COX-2 induction. (F) Positive control: additional of human recombinant IL-1β (1 ng/mL+5 μg IgG1 isotype). (G) human recombinant IL-1β pre-incubated with 5 μg/mL neutralizing antibody. Results are shown as fold induction of COX-2 mRNA relative to group A. In conclusion, this experiment demonstrated that activation of the IL-1β^{XAT} gene results in production of biologically potent IL-1β and subsequently up-regulation of the inducible COX-2. (N=3). *p<0.05; S.E.M.

FIG. 5 shows Cre-mediated activation of the COLL1-IL-1β^{XAT} gene. The 3.6 Kb promoter of the A1 chain of pro-collagen I gene, which has been shown to target gene expression in bone and cartilage, drove the expression of the IL-1β^{XAT} gene in NIH 3T3 stable cell line following transfection with the pRc/CMV-CreWT vector and infection with the HIV(Cre) virus. Panel (A) depicts transfection (+) of such a stable cell line with pRc/CMV-CreWT, leading to expression of human IL-1β expression concomitantly with Cre recombinase as detected by RT-PCR. In contrast, untreated cells (−) were characterized by the absence of IL-1β and Cre recombinase. Panel (B) depicts similar IL-1β and Cre expression as assessed by RT-PCR following infection of the COLL1-IL-1β^{XAT} cell line with the HIV(Cre) virus. The presence of IL-1β^{XAT} in the cells was confirmed by PCR as shown.

FIG. 6 shows that IL-1β induces inflammation-related genes. The effects of IL-1β were evaluated in vitro utilizing primary rat endothelial cell cultures as a representative rodent cell type. In this experiment, murine IL-1β (10 ng/mL) was administered exogenously to cultured primary cells, and subsequently examined the regulation of several inflammation-related genes at the transcript level over the course of 72 hours. These molecules include (A) the inducible isoform of cyclooxygenase (COX-2), intercellular adhesion molecule-1 (ICAM-1) and monocyte chemoattractant protein-1 (MCP-1), as well as (B) the collagenase-A (MMP-2) and -B (MMP-9). Panel (C) depicts enzyme activity levels of MMP-2 and MMP-9 as evaluated by zymography.

FIG. 7 shows that recombinant IL-1β induced transcriptional expression of (A) intercellular adhesion molecule-1 (ICAM-1), monocyte chemoattractant protein-1 (MCP-1), and (B) inducible collagenase-B (MMP-9) as assessed at the mRNA level by RT-PCR in rodent endothelial cells in vitro. As anticipated, enzyme activity for both collagenase-A (MMP-2) and -B (MMP-9) was upregulated by IL-1β as assessed by zymography. Interestingly, simultaneous administration of the representative NSAID indomethacin (INDO)+IL-1β resulted in exacerbation of the effects elicited by IL-1β alone. Specifically, ICAM-1, MCP-1 (A) and MMP-9 (B) transcript levels were further increased by INDO, as was collagenase activity for both MMP-2 and MMP-9 (C). The regulation of pro-collagen IV was also included in the study as control (not an inflammatory gene)—Panel B.

FIG. 8 shows resistance to mouth opening as a behavioral measure. Patients with TMJ dysfunction and pain are characterized by a common array of clinical features, including limitation of jaw opening and increased pain from jaw function (see discussions herein). Methods were adopted based on these principles for the assessment of symptoms from the TMJ. (A) The mice have orthodontic hooks fixed on the upper and lower incisor teeth with light cure orthodontic resin; the maxillary hook stabilizes the upper jaw vertically, whereas the lower hook is attached to an electronic dynamometer (B). The lower hook with the dynamometer are lowered at predefined vertical distances (5 mm, 10 mm, 15 mm, 20 mm and 25 mm) and the resistance to jaw opening is recorded. (C) Previous experiments in mice have demonstrated that the animal will attempt to close the mouth when the mandible is depressed. This type of experiment was performed on TNFα transgenic mice displaying TMJ rheumatoid arthritis at 12 weeks. There was a 75% reduction in closing bite force compared to controls. The data represent the mean±standard deviation. Five animals were tested three times and the mean and S.D. of resistance to mouth opening were determined. ** P<0.01.

FIG. 9 shows electromyography of the masticatory muscles in evaluating TMJ pain. Electromyography signals were obtained with a telemetry system using a fully implantable device that combines continuous registration of one biopotential (right masseter muscle). The implant (ETA-F20, Data Sciences International—DSI, St. Paul, Minn.) consists of an electronics module and a battery, which transmits data for at least 6 months with a magnetically activated on-off switch, extending battery life. Extending from the silicone housing are two flexible leads, i.e. bipolar electrodes and one ground lead. Each 40 mm lead contains a helix of stainless steel wire (diameter: 0.45 mm) with an insulating layer of silicone tubing (diameter: 0.8 mm). The 45×17×10 mm implant is biocompatible, sealed, sterile and calibrated. Within the implant, the biopotentials are filtered (first order low-pass filter, 158 Hz; personal communication, DSI) and sampled (5,000 Hz) and using a carrier frequency (455 kHz), the output is transferred to the transmitter leads. A receiver is placed under the cage (RMC-1, 31×24.5×3.5 cm, DSI) that collects this signal and the extracted data strings are saved on hard disk using the Dataquest A.R.T. data acquisition system (DSI). For the F20 implant device, specification of the maximum cage size is 42×42×18 cm (using a single receiver). Panel A represents a 10 second sample representing 50,000 data points. The individual chewing strokes that represent the highest generated muscle activity can be identified as a peak utilizing the mathematical procedure of the moving window criteria. Panel B represents 3 chewing strokes from Panel A that have been isolated and rectified. Each maximal peak represents the 100 percent activity (maximal activity) of the individual chewing stroke. These data can be rectified, and a moving average can be used to smooth the curves. From these, the ascending and descending 50% and 25% activities can be established simultaneously (C). The integrated signal from the first chewing motion in panel B was analyzed. The area of the chewing stroke activity was identified utilizing Simpson's rule in which the area is approximated by parabolas. Peak activity is identified at 100% and the corresponding 25% and 50% of peak activity for ascending and descending curve can be calculated. Times and areas are simultaneously calculated for each period. The skewness and kurtosis of these individual curves can be calculated and analyzed individually and as an average for the five 10 seconds periods of EMG. Statistical analysis includes the following process. Multiple chewing strokes can be used to evaluate the reliability of the time and amplitude of the EMG signal using intraclass correlation coefficient. There are about 25 chewing strokes for each 10 seconds of sampling and the EMG signal (Panel A) and the chewing of a cheerio can be recorded for one minute. The ascending and descending 50% and 25% activities and maximal activity can be used for the analysis as well as the times to reach each time point.

FIG. 10 shows FIV(Cre): a self-inactivating feline immunodeficiency viral vector. A custom nlscre transgene was constructed and cloned in the FIV transfer vector flanked by loxP sites. FIV virus is produced in vitro by co-transfecting the recombinant FIV transfer vector into 293H cells along with the packaging and viral envelop (VSV-G) vector.

FIG. 11 shows recombination with the IL-1β XAT construct. Equimolar mixtures of IL-1β XAT (CMV promoter) and pRC/CMV or pRC/CMV-cre (2 μg total plasmid DNA) were transfected into 293H cells (Invitrogen, Carlsbad, Calif.) in 12 well plates using Lipofectamine 2000 (Invitrogen). 72 hours following transfection, cells were fixed and stained for lacZ activity using X-gal histochemistry.

FIG. 12 shows FIV-lacZ transduces murine astrocytes in vitro. Murine astrocyte cultures at approximately 60% confluence were infected with the FIV-lacZ vector using a multiplicity of infection (MOI) of 1. Staining for lacZ expression in fixed cells was carried out using X-gal histochemistry at 48 hours.

FIG. 13 shows that the IL-1^{XAT} Construct is composed of a 2.2 kb human GFAP promoter, a loxP element (red), three exons from the human growth hormone, one of which has a frameshift mutation (FS) to disrupt the open reading frame, a β-globin translational terminator, a second loxP element, the signal sequence from hIL-1ra (ss) fused in frame to the coding sequence for mature human IL-1β, an internal ribosomal entry site (IRES) followed by coding sequence for β-galactosidase, and 3′ flanking DNA from the human growth hormone. Following cre recombinase mediated excision, the β-globin terminator was removed, allowing transcription of sshIL-1β and lacZ.

FIG. 14 shows that FIV production is accomplished in vitro following co-transfection of the aforementioned vectors into 293-T cells. The FIV-rich supernatant is then collected, filtered and can be used directly or following concentration by centrifugation. Titers routinely range between 107-108 infectious particles/mL. Although pFIV(lacZ) is shown in this illustration, pFIV-gfp and pFIV-cregfp have been made also.

FIG. 15 shows a map of the pGFGH plasmid containing the 2.2 kbp murine GFAP promoter. The promoter was excised using EcoRI and NotI restriction enzymes.

FIG. 16 shows a map of the linearized IL-1β^{XAT} construct (analogous to RAP^{XAT}, except for the substitution of hIL-1RA for ssIL-1β).

FIG. 17 shows recombination and gene induction in a rat astrocyte stable cell line (RBA2) expressing RAP^{XAT} under control of the GFAP promoter. Stable cell lines were established after transfection with RAP^{XAT} and selection with G418. 1=Naïve cells; 2=Transfection with Prc/CMV; 3=Transfection with Prc/CMV-Cre; 4=Viral Infection with FIVCre. a) LoxP DNA recombination; b) IL-1RA protein concentrations in media determined by ELISA; c) X-gal histochemistry.

FIG. 18 shows identification of 2 IL-1β^{XAT} transgenic founders using primers flanking the 5′ and 3′ ends of the ssIL-1β transgene (HIL-1B-FIXUP and IL-1B-17 kD-LP), producing a 539 bp PCR product in transgenic positive mice.

FIG. 19 shows identification of 3 RAP^{XAT} transgenic founders using primers flanking the 5′ and 3′ ends of the hIL-1RA transgene (HIL-1B-FIXUP and HSIL-1RA-LP), producing a single 534 bp PCR product in transgenic positive mice.

FIG. 20 shows identification of 6 RAP^{XAT} F1 transgenic mice (all offspring of mouse 786-5-4 bred with a wild-type mouse). Primers used are identical to FIG. 5 (HIL-1B-FIXUP and HSIL-1RA-LP).

FIG. 21 shows the Cre-mediated activation of the COL1-IL1β^{XAT} gene. The 3.6 Kb promoter of the A1 chain of pro-collagen I gene, which has been shown to target gene expression in bone and cartilage, drove the expression of the IL-1^{XAT} gene in NIH 3T3 stable cell line following transfection with the HIV(Cre) vector and also infection with the HIV(Cre) virus. Panel (A) depicts transfection (+) of such a stable cell line with HIV(Cre) vector, leading to expression of human IL-1β expression (IL-1β RT-PCR) concomitantly with Cre recombinase (Cre RT-PCR) as detected by RT-PCR at the mRNA transcript level. In contrast, untreated cells (−) were characterized by the absence of IL-1β and Cre recombinase. Panel (B) depicts similar IL-1β and Cre expression as assessed by RT-PCR following infection of the COL1-IL1^{XAT} stable cell line with the HIV(Cre) virus. The presence of the IL-1^{XAT} transgene in the cells was confirmed by PCR as shown (IL-1β^{XAT}).

FIG. 22 shows the development of COL1-IL1β^{XAT} cell lines. Five COL1-IL1β^{XAT} clones were picked and expanded based on the presence of the IL-1B^{XAT} transgene in their genomic DNA. (A) The presence of the IL-1B^{XAT} transgene was confirmed by DNA amplification of the ssIL1β sequence using the HIL-1B-FIXUP and IL1B-17kD-LP primers (described in section A). As positive control for the PCR reaction, the pCOL1-IL1β^{XAT} vector was employed (cntl). (B) The induction of COL1-IL1β^{XAT} was evaluated in cells that were transfected with the HIV(Cre) vector. Cell line #2 and #3 were positively identified. (C) The expression of Cre recombinase in these conditions was confirmed by Cre RT-PCR. These cell line have also named as follows based on their derivation: Line #1=>naïve cells; Line #2=>9-2 cell line; Line #3=>9-3 cell line; Line #4=>9-4 cell line; Line #5=>13-1 cell line.

FIG. 23 shows FIV(nlsCre) viral vector development and induction of COL1-IL1β^{XAT} in vitro. Packaged FIV(nlsCre) virus was used to infect NIH 3T3 cells, which were subsequently transfected with the CMV-IL1BXAT genes. Activation of the dormant gene was evaluated by lacZ expression as assessed by X-gal histochemistry (blue cells).

FIG. 24 shows COL1-IL-1β^{XAT} transgenic mouse lines. The University of Rochester Transgenic Animal facility has performed a series of microinjections, which yielded 3 strong candidate transgenic COL1-IL-1β^{XAT} mouse lines: #4, #11 and #12. This figure depicts PCR amplification of the transgene using a set of primers that also amplify the endogenous murine IL-1β gene at low levels. Transgene transmission in the offspring of #4, 11 and 12 transgenic founders is analyzed. c=control (C57B1/6 stock); 4, 11, 12, 13, 14=Transgenic mouse lines; +=PCR positive control; −=PCR primers control.

FIG. 25 shows COL1-IL-1β^{XAT} transgenic mouse lines: Colony Status.

FIG. 26 shows COL1-IL-1β^{XAT} transgenic mouse: Founder #4—detail of the line.

FIG. 27 shows behavioral changes in Col1-IL1β^{XAT} mice after injection of FIV(Cre) in the knees. A group of Col1-IL1β^{XAT} transgenic mice (N=3) received a single intra-articular injection of 10⁶ infectious particles of FIV(Cre) in the right and left knees at 2 months of age. In addition, a second group of mice (N=3) received saline injection and served as controls. During a session, each mouse was videotaped for 1 hour. The tape was then transferred digitally to a computer and analyzed in 20 periods of 3 minutes each. The duration of each mouse displaying grooming and licking was recorded and summed as seconds. The analysis of the behaviors was made by an investigator who was blind to the animal group assignment. Statistical analysis was performed by t-Test. Error bars=SEM. *=P<0.05.

FIG. 28 shows locomotive deterioration in Col1-IL1β^{XAT} mice after injection of FIV(Cre) in the knees. Four groups of mice (N=3) were evaluated in terms of locomotive behavior by the rotorod appliance (Columbus Instruments, Columbus Ohio) and the lapse time until the mice well off the rotating cylinder (20 rpm) was recorded. The mice were evaluated over a period of 8 weeks following the intra-articular injections (8 wks-16 wks of age).

FIG. 29 shows FIV(Cre) injection in the knee of Col1-IL1β^{XAT} mice resulted in transgene induction. Immunocytochemical detection of the reporter gene β-galactosidase was employed to confirm the activation of the Col1-IL1β^{XAT} transgene by FIV(Cre) in this mouse model using antibodies raised against β-galactosidase and Cre recombinase. (A) FICT-conjugated immunodetection of β-galactosidase, (B) Texas Red-conjugated immunodetection of Cre recombinase, and (C) B/W image of the same microscopic field. (D) Overlap of panels A+B, and (E) overlap of panels A+B+C demonstrating co-expression of β-galactosidase and Cre recombinase in vivo (solid arrows). Note that there are more red cells than green cells (open arrows) indicating that not all infected cells express the transgene Col1A1→IL1β-IRES-lacZ in the same capacity. All images were captured at a magnification of 20×. “m”=meniscus; “a”=articular surface; “I”=intra-articular space.

FIG. 30 shows arthritic changes in the knee joint of Col1-IL1β^{XAT} mice following injection of FIV(Cre). (A) H&E staining of a knee section harvested from a 4 month old Col1-IL1β^{XAT} transgenic mouse injected with FIV(Cre) revealed the formation of fibrillations (solid arrow) and of an articular lip (open arrow). In contrast, (B) a transgenic mouse that received the control vector FIV(GFP) did not develop such anatomic aberrations. (C) Alcian blue/orange semi-quantitative evaluation showed a decrease in cartilage (less blue stain) and bone (less red stain) density in the Col1-IL1β^{XAT}+FIV(Cre) knees compared to (D) controls. Moreover, increased cloning along with thickening of the articular surfaces was observed in the experimental animals (indicated by small arrows).

FIG. 31 shows brain inflammation in Col1-IL1β^{XAT} mice following injection of FIV(Cre) in the knee and TMJ. Eight weeks after FIV(Cre) injection in the knee and TMJ of Col1-IL1β^{XAT} mice we evaluated the brain for activation of microglia and astrocytes by immunocytochemistry. (A) Using a monoclonal antibody raised against the MHC-class II antigen, we detected the presence of activated microglia in the brain. In contrast, control animals did not display any MHC-II positive cells. (C) Larger magnification of panel A. (B) There was lack of astrocyte activation in the brains of these animals as assessed by glial fibrillary acidic protein (GFAP). (D) Larger magnification of panel B.

FIG. 32 shows arthritis-like changes in the TMJ of Col1-IL1β^{XAT} mice after intra-articular injection of FIV(Cre). Eight weeks after FIV(Cre) injection in the TMJ of Col1-IL1β^{XAT} mice we evaluated anatomic aberrations of the joint by semi-quantitative Alcian blue—orange G histochemistry. (A) TMJ section from an inactive Col1-IL1β^{XAT} mouse depicting the condylar head as well as the meniscus. In comparison, (B) a TMJ section harvested from a Col1-IL1β^{XAT} mouse injected with FIV(Cre) in the TMJ. (C) Larger magnification of the identified area of panel A. (D) Larger magnification of the identified area of panel B.

FIG. 33 shows GFP expression in the mouse hippocampus 1 week following FIV-GFP injection. Coordinates: 1.8 mm lateral and caudal to bregma, 1.7 mm deep to brain surface.

FIG. 34 shows glial activation following hIL-1β induction. ICC was performed 2-weeks following FIV-Cre injection in heterozygous IL1b-XAT (Lines A/a and B/b) or wild-type (WT) mice. There is robust upregulation of MHC-II and GFAP (B/b>A/a) as compared to wild-type animals.

FIG. 35 shows inflammatory marker upregulation. ICC was performed 2-weeks following FIV-Cre or FIV-LacZ injection in heterozygous IL1b-XAT mice (Lines A/a and B/b). ICAM-1 and MCP-1 are markedly upregulated compared to FIV-LacZ injected line B/b control animals.

FIG. 36 shows neutrophil recruitment to the mouse hippocampus. 2-weeks following FIV-Cre injection there were numerous neutrophils recruited to the hippocampal parenchyma (B/b>>A/a) as evidenced by 7/4 antibody staining. Parenchymal 7/4 antibody staining in absent in wild-type (WT) mice.

FIG. 37 shows time course of gene transcript induction. IL1b-XAT line B/b demonstrates significant upregulation of MHC-II, GFAP and MCP-1 in the ipsilateral hemisphere extending up to 4 weeks after gene activation. IL1b-XAT line A/a shows a milder, distinct phenotype. i=ipsilateral; c=contraleral. **=p<0.1; ***=p<0.001.

FIG. 38 shows upregulation of the ELR+CXC chemokines. 2 weeks following IL-1β induction there is significant upregulation of KC and MIP-2 in line B/b. These chemokines are members of the neutrophil chemoattractant ELR+CXC chemokine family. CXCR2 receptor expression is also significantly increased in line B/b, likely due to expression by infiltrating neutrophils.

FIG. 39 shows orofacial grooming as a behavioral measure of formalin-induced TMJ pain. Intra-articular TMJ injection of formalin (10 μL of 0.625% formalin in saline) in 2 month old male C57BL/6 mice resulted in significantly increased orofacial grooming (TMJ-F) compared to mice receiving saline (TMJ-S) or no injection (CNTL). Pre-treatment of the mice with morphine (intraperitoneal administration 30 min prior to formalin injection) resulted in attenuation of orofacial grooming in formalin-challenged mice to near normal levels. N=5; *P<0.05

FIG. 40 shows resistance to mouth opening as a behavioral measure of formalin-induced TMJ pain. Intra-articular TMJ injection of formalin (10 μL of 0.625% formalin in saline) in 2 month old C57BL/6 male mice resulted in significantly decreased resistance to mouth opening (TMJ-F) compared to mice receiving saline (TMJ-S) or no injection (CNTL) 90 min after the formalin injection. Moreover, pre-treatment of mice with morphine (intraperitoneal administration 30 min prior to formalin injection) resulted in attenuation of orofacial grooming in formalin-challenged mice to near normal levels (F+MOR). N=5; *P<0.05.

FIG. 41 shows FIV(Cre) injection in the knee of Col1-IL1β^{XAT} mice resulted in transgene induction. Immunofluorescent detection of the reporter gene β-galactosidase was employed to confirm the activation of the Col1-IL1β^{XAT} transgene by FIV(Cre) in this mouse model using antibodies raised against β-galactosidase and Cre recombinase. Panel (A) depicts FITC-conjugated immunodetection of β-galactosidase, (B) Texas Red-conjugated immunodetection of Cre recombinase. (C) Overlap of panels A+B, and (D) overlap of panel C with respective dark field image demonstrating histology. Note that not all infected cells express the IL1β^{XAT} transgene in the same capacity. All images were captured at a magnification of 100×.

FIG. 42 shows FIV(Cre) injection in the knee of Col1-IL1β^{XAT} mice resulted in chronic expression of hIL-1β. Immunohistochemical analysis of knee sections harvested from adult Col1-IL1β^{XAT} transgenic mice 8 weeks following intra-articular injections of (A) FIV(Cre) and (B) FIV(gfp). A commercially available antibody (Abcam cat. No. ab2105; Cambridge, Mass.) raised against recombinant human mature IL-1β that does not cross-react with the murine or rat cytokine was employed (black staining). (40×) c-cartilage; i-intra articular space; p-pannus.

FIG. 43 shows arthritic changes in the knee joint of Col1-IL1β^{XAT} mice following injection of FIV(Cre). (A) Alcian blue—orange G staining of a knee section harvested from a 4 month old Col1-IL1β^{XAT} transgenic mouse injected with FIV(Cre) compared to (B) a control mouse (littermate Col1-IL1β^{XAT} transgenic mouse) injected with FIV(gfp). This revealed the formation of multiple fibrillations. Also, there is appreciable cartilage erosion and loss of the resting chondrocyte layer in experimental Col1-IL1β^{XAT} mice accompanied by remodeling of subchondral bone. (C) IL-6 expression, a marker of joint inflammation, was found upregulated by immunohistochemistry in experimental Col1-IL1β^{XAT} mice compared to (D) controls. (40×).

FIG. 44 shows Col1-IL1β^{XAT} gene activation in the TMJ of transgenic mice by FIV(Cre) injection. Immunofluorescent detection of the reporter gene β-galactosidase was employed to confirm the activation of the IL1β^{XAT} transgene in the TMJ by FUV(Cre) in this mouse model. (A) FITC-conjugated detection of β-galactosidase, (B) Texas Red-conjugated detection of Cre, and (C) B/W image of the same microscopic field. (D) Overlap of panels A+B, and (E) overlap of panels A+B+C demonstrating co-expression of β-galactosidase and Cre recombinase in vivo. Note that there are more red cells than green cells indicating that not all infected cells express the transgene Col1A1->IL1β-IRES-lacZ in the same capacity. (100×).

FIG. 45 shows long term expression of human IL-1β protein in the TMJ of activated Col1-IL1β^{XAT} mice. Immunohistochemical analysis of TMJ sections harvested from adult Col1-IL1β^{XAT} transgenic mice 8 weeks following intra-articular injections of (A) FIV(Cre) and (B) FIV(gfp). A commercially available antibody (Abcam cat. No. ab2105; Cambridge, Mass.) raised against recombinant human mature IL-1β that does not cross-react with the murine or rat cytokine was used (black staining). c-condyle; i-intra articular space; d-articular disc.

FIG. 46 whose COL1-IL1β^{XAT} activation in the TMJ induces the expression of inflammatory mediators. Eight weeks following FIV(Cre) injection in the TMJ of Col1-IL1β^{XAT} transgenic mice, the TMJ's of (A-C-E) control (Tg+gfp) and (B-D-F) experimental (Tg+Cre) were harvested and evaluated by immunocytochemistry using antibodies against murine IL-6, COX-2 and MMP-9. A total of 20 COL1-IL1β^{XAT} transgenic mice and 16 wild type littermates were employed in this experiment. (A,B) Induction of IL-6 was observed in the proliferative zone of the articular surface, as well as (C-D) increased COX-2 expression (red staining). Moreover, MMP-9 (gelatinase B) was also found increased in the experimental mice compared to controls (E-F) as assessed by immunohistochemistry (red stain—hematoxylin nuclear counter stain). Induction of IL-6, COX-2 and MMP-9 indicated the presence of inflammation in the TMJ of adult activated transgenic mice (40×).

FIG. 47 shows arthritic changes in the TMJ. (A) The number of cells staining positive for COX-2, IL-6 and MMP-9 were counted in TMJ immunohistochemistry sections. A total of 20 Col1-IL1β^{XAT} transgenic mice and 16 wild type littermates were employed in this experiment. Experimental mice (Tg+Cre) showed higher numbers of immunoreactive cells than controls (Tg+gfp) at a statistically significant degree. (B) In addition, COX-2 and iNOS transcript levels were increased in the experimental group compared to controls as assessed by quantitative RT-PCR in TMJ total RNA extracts. (C) Cartilage loss at the articular surface of the joints was scored on a scale 0-5. (D) Chondrocyte cloning in the articular cartilage was assessed in experimental and control mice. N=5; **p<0.01; *p<0.05 Mean+/−S.D.

FIG. 48 shows Col1-IL1β^{XAT} activation in the adult TMJ results in orofacial pain and joint dysfunction. (A) Pain was evaluated by assessing orofacial grooming in adult transgenic mice after a period of 8 weeks following transgene activation. Transgene activation by FIV(Cre) (N=6) intra-articular injections in the TMJ resulted in increased levels of grooming behavior compared to FIV(gfp)-(N=5) or saline-injected (N=4) Col1-IL1β^{XAT} transgenic mice. (B) Joint dysfunction was evaluated by assessing resistance to jaw opening. FIV(Cre) injected transgenic mice demonstrated significantly decreased levels of resistance to vertical mandibular displacement. (C) CGRP expression in the trigeminal ganglia of experimental and control mice was calculated as total immunoreactivity in 4× fields and presented as relative % ratio. (D) RCP expression was assessed by immunohistochemistry in brain stem sections and calculated as total immunoreactivity in 20× fields. (E) Representative section (40×) of a trigeminal ganglion of a Col1-IL1β^{XAT} transgenic mouse injected with FIV(Cre) stained for CGRP. (F) Representative section (40×) of RCP (receptor complimentary protein, inducible component of the CGRP receptor previously associated with inflammatory pain) immunostaining in the principal trigeminal nucleus of a Col1-IL1β^{XAT} transgenic mouse injected with FIV(Cre). *p<0.05. Bar-100 μm.

FIG. 49 shows murine IL-1β is induced in the brain stem of mice suffering from chronic TMJ arthritis. The level of murine IL-1β, was analyzed at the protein level in the brain stem of Col1-IL1β^{XAT} transgenic mice suffering from chronic TMJ arthritis. In brief, transgene expression was induced by FIV(Cre) intra-articular injection in the TMJ of adult transgenic mice. Eight weeks following viral transduction, the level of murine IL-1β expression was found significantly increased at the level of the main sensory nuclear of their brain stem compared to FIV(gfp)-injected (control) mice.

FIG. 50 shows astrocyte activation in the brain stem of Col1-IL1β^{XAT} mice exhibiting TMJ arthritis and pain. Astrocyte activation, as assessed by GFAP IHC, was observed in the brain stem of Col1-IL1β^{XAT} transgenic mice 8 weeks following the induction of TMJ arthritis. Specifically, the activated astrocytes were located proximally to the IL-1β induction at the main sensory nucleus of mice suffering from TMJ arthritis and orofacial pain. (A) Base-line GFAP staining in control mouse (Tg+gfp). (B) Increased GFAP expression was observed in experimental mice (Tg+Cre). (C) Higher magnification of panel B.

FIG. 51 whose IL-1β injection into the cisterna magna induces neuronal excitation and astrocyte activation. Recombinant IL-1β was injected into the cisterna magna of adult mice (10 ng in 2 μl of aqueous solution). Neuronal excitation was evaluated by CGRP and astrocyte activation by GFAP IHC. (A) The saline-injected mouse is characterized by lack of CGRP expression at the main sensory nucleus. Conversely, (B) the IL1β-injected mice displayed pronounced CGRP immunoreactivity at the level of the main sensory nucleus. Furthermore, activation of astrocytes by GFAP upregulation was observed at the same brain stem level. GFAP immunohistochemistry in (C) control and (D) experimental mice.

FIG. 52 shows FIV(IL1ra) successfully transduces cells with a gene expressing IL-1ra receptor antagonist. FIV(IL1ra) was constructed as depicted in panel A and confirmed by restriction enzyme analysis depicted in panel B. FIV(IL1ra) was then tested in vitro; IL1ra expression was evaluated in murine NIH 3T3 cells infected with this virus at the mRNA and protein levels. (C) RT-PCR analysis of infected cells demonstrated the expression of IL1ra mRNA. In contrast, naïve cells did not display any IL1ra expression. The housekeeping gene G3PDH was also employed. (D) IL1ra protein level in the media of injected cells was assessed by ELISA. Infection of cells by FIV(IL1ra) resulted in therapeutic IL1ra levels (>30 μg/mL). In contrast, FIV(gfp) and naïve cells did not express IL1ra.

### DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

“Primers” are a subset of probes which are capable of supporting some type of enzymatic manipulation and which can hybridize with a target nucleic acid such that the enzymatic manipulation can occur. A primer can be made from any combination of nucleotides or nucleotide derivatives or analogs available in the art which do not interfere with the enzymatic manipulation.

“Probes” are molecules capable of interacting with a target nucleic acid, typically in a sequence specific manner, for example through hybridization. The hybridization of nucleic acids is well understood in the art and discussed herein. Typically a probe can be made from any combination of nucleotides or nucleotide derivatives or analogs available in the art.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular vector is disclosed and discussed and a number of modifications that can be made to a number of molecules including the promoters are discussed, specifically contemplated is each and every combination and permutation of vectors and promoters and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

### B. COMPOSITIONS

Provided herein are compositions and methods for the temporally and spatially-regulated transgene expression of inflammation related molecules, such as IL-1β or its antagonists. An inflammation related molecule is a molecule that is involved in an inflammation signal transduction pathway. Inflammation related molecules in these pathways can promote or inhibit inflammation. The provided Cre/loxP molecular genetic methods utilize a germline-transmitted recombinational substrate containing a dormant transcription unit and somatic gene transfer of a viral vector that expresses Cre recombinase to activate the gene of interest. Gene activation is accomplished by a recombinant self-inactivating vector expressing Cre. Recombination-mediated gene “activation” permanently alters the genetic constitution of infected cells thus allowing chronic IL-1β expression.

Provided herein are compositions and methods for the generation of animal models of inflammatory disease. The animal models provided herein comprise temporally- and/or spatially-regulated transgenic expression of inflammatory mediators. It is understood that the compositions and methods provided herein can be applied to any mediator of inflammation. Examples of inflammatory mediators include COX, such as COX-2, IL-1, such as IL-1β, IL-1ra.

Disclosed are methods and compositions related to vectors, cells, transgenic animals, and methods thereof that provide a model(s) to explore inflammation, such as the contribution of IL-1 to neurodegenerative disorders and other neurological conditions such as stroke and traumatic brain injury as well as joint disorders, such as arthritis. Provided are transgenic mice that harbor transcriptionally silent transgenes for IL-1β and its native antagonist, IL-1ra, where the transgenes can be turned on in a cell specific or temporally specific manner. Further provided are viral vectors expressing cre recombinase, wherein sustained expression of the transgenes can be initiated at a selected age and in a specific region of brain. Further provided are methods to conditionally and regionally secrete IL-1β or IL-1ra within specific tissues, allowing studies of chronic elevation of these cytokines without confounding issues of compensatory changes during development.

A particular advantage of the provided compositions and methods is the herein described ability to activate the herein described transgenes in the brain by means of peripheral administration. For example, FIV vectors are disclosed herein that can deliver the herein disclosed nucleic acids (e.g. Cre Recombinase) to target sites within the subject. The disclosed FIV constructs can be delivered systemically by injection into the circulation or locally by injection into the target site, such that either method of administration can result in the delivery of the nucleic acid to cells in the brain, such as, for example, microglia or astrocytes. The use of FIV vectors to deliver nucleic acids or transgenes to the brain following systemic administration is described in Patent Cooperation Treaty Application No. PCT/US03/13672 and U.S. Provisional patent application Ser. No. 10/781,142, which are herein incorporated by reference in their entirety as they related to this teaching.

1. Joint Disorders and IL-1β

Inflammatory mediators, including pro-inflammatory cytokines and prostanoids, such as interleukin-1β (IL-1β) and prostaglandin E₂ (PGE₂), have also been implicated in temporomandibular joint disorders (TMJD) pathology in humans. Clinically, orofacial pain is frequently associated with disturbances in somatosensory and jaw motor function, such as pain during mastication. Of concern is the possibility that tissue injury caused by trauma, disc displacement, mechanical stress, infection or iatrogenic procedures results in chronic expression of pro-inflammatory cytokines in the temporomandibular joint that ultimately can lead to arthritis, hyperalgesia and tissue degeneration.

a) Arthritis

Arthritis as a disease can include many different disorders and symptoms and can affect many parts of the body. Arthritis typically causes pain, loss of movement and sometimes swelling.

Arthritis is actually a term used for a set of more than 100 medical conditions. Arthritis is most commonly associated with older individuals, but can start as early as infancy. Some forms affect people in their young-adult years.

A common aspect among arthritic conditions is that they affect the musculoskeletal system and specifically the joints—where two or more bone meet. Arthritis-related joint problems can include pain, stiffness, inflammation and damage to joint cartilage (the tough, smooth tissue that covers the ends of the bones, enabling them to glide against one another) and surrounding structures. Such damage can lead to joint weakness, instability and visible deformities that, depending on the location of joint involvement.

Many of the arthritic conditions are systemic, in that they affect the whole body. In these diseases, arthritis can cause damage to virtually any bodily organ or system, including the heart, lungs, kidneys, blood vessels and skin.

Some different types of arthritis are Osteoarthritis, Rheumatoid arthritis, Gout, Ankylosing spondylitis, Juvenile arthritis, Systemic lupus erythematosus (lupus), Scleroderma, and Fibromyalgia.

Osteoarthritis is a degenerative joint disease in which the cartilage that covers the ends of bones in the joint deteriorates, causing pain and loss of movement as bone begins to rub against bone. It is the most prevalent form of arthritis.

Rheumatoid arthritis is an autoimmune disease in which the joint lining becomes inflamed as part of the body's immune system activity. Rheumatoid arthritis is one of the most serious and disabling types, affecting mostly women.

Gout affects mostly men. It is usually the result of a defect in body chemistry. This painful condition most often attacks small joints, especially the big toe. Fortunately, gout almost always can be completely controlled with medication and changes in diet.

Ankylosing spondylitis is a type of arthritis that affects the spine. As a result of inflammation, the bones of the spine grow together.

Juvenile arthritis is a general term for all types of arthritis that occur in children. Children may develop juvenile rheumatoid arthritis or childhood forms of lupus, ankylosing spondylitis or other types of arthritis.

Systemic lupus erythematosus (lupus) is a disorder that can inflame and damage joints and other connective tissues throughout the body.

Scleroderma is a disease of the body's connective tissue that causes a thickening and hardening of the skin.

Fibromyalgia is a disorder in which widespread pain affects the muscles and attachments to the bone. It affects mostly women.

2. Neuroinflammation and IL-1β

Neuroinflammation, characterized by activated microglia and astrocytes and local expression of a wide range of inflammatory mediators, is a fundamental reaction to brain injury, whether by trauma, stroke, infection, or neurodegeneration. This local tissue response is surely part of a repair and restorative process. Yet, like many inflammatory conditions in peripheral diseases, neuroinflammation can contribute to the pathophysiology of CNS disorders. For example, in Alzheimer's disease (AD), glial-driven inflammatory responses to Aβ deposition are thought to promote neurodegeneration, as evidenced by the extent of neuroinflammation in AD, increased risk for AD with certain polymorphisms of proinflammatory cytokine genes, and reduction in disease risk for individuals taking nonsteroidal anti-inflammatory drugs (NSAIDs).

Likewise, inflammatory processes are thought to be involved in various arthritic conditions of the joints, such as osteoarthritis.

IL-1 is a potent immunomodulating cytokine that exists as two principal isoforms, IL-1α and IL-1β. These two molecules show significant divergence in sequence and have somewhat different roles with IL-1α generally thought to be involved in direct cell:cell communication whereas IL-1β is secreted. Nevertheless, these two molecules act through the same membrane-associated receptor known as IL-1 receptor type 1 (IL-1R1) to promote a proinflammatory signaling cascade that includes the activation of NFκB and MAP kinases [Rothwell, N. J. and G. N. Luheshi. Trends Neurosci. (2000) 23:618-625]. Interestingly, at least two molecules have been identified that antagonize the effects of IL-1: IL-1 receptor antagonist (IL-1ra) competes for receptor binding, and IL-1 receptor type 2 (IL-1R2) lacks an intracellular domain and is thought to serve as a decoy receptor [Rothwell, N. J. and G. N. Luheshi. Trends Neurosci. (2000) 23:618-625]. Expression of each of these molecules is regulated. Thus, the contribution of IL-1 to an inflammatory response depends on the relative balance of expression between these family members [Arend, W. P. Cytokine & Growth Factor Rev. (2002) 13:323-340].

Although both isoforms of IL-1 are made in brain, most work has focused on the role of IL-1β. Principally produced by microglia, IL-1β is rapidly induced following CNS injury. IL-1β affects many cellular targets, including astrocytes, neurons, and endothelial cells. In these cells, IL-1 up-regulates cytokines and chemokines, induces the expression of cell surface adhesion molecules and matrix metalloproteases, and stimulates cell proliferation [St Pierre, B. A., et al. Effects of cytokines on CNS cells: glia, in: (Ed.) Ransohoff, R. M., E. N. Beveniste, Cytokines and the CNS, CRC Press, Boca Raton, (1996) pp. 151-168]. Moreover, it has been demonstrated that IL-1β induces cyclooxygenase-2 (COX-2) in brain astrocytes, leading to production of the proinflammatory prostaglandin PGE₂ [O'Banion, M. K., et al. Neurochem. (1996) 66:2532-2540]. Taken together, the myriad effects of IL-1 on multiple brain cell types suggest a critical role for IL-1 family members in coordinating brain neuroinflammatory responses.

The profound influence of IL-1 on neuroinflammation and its ubiquitous expression in conditions ranging from frank brain trauma to neurodegenerative disease suggests that it might contribute to CNS injury [Rothwell, N. J. and G. N. Luheshi. Trends Neurosci. (2000) 23:618-625]. This appears to be the case. For example, IL-1β is induced in experimental models of stroke [Minami, M., K. et al. J. Neurochem. (1992) 58:390-392] and infusion of IL-1β exacerbates damage whereas treatment with IL-1ra or IL-1 blocking antibodies significantly attenuates tissue injury [Loddick, S. A. and N. J. Rothwell. J. Cereb. Blood Flow Metab. (1996) 16:932-940 and Yamasaki, Y., N. Matsuura, H. Shozuhara, H. Onodera, Y. Itoyama and K. Kogure. Stroke (1995) 26:676-681]. Similarly, ischemic injury is significantly attenuated in interleukin-1 converting enzyme deficient mice [Friedlander, R. M., et al. J. Exp. Med. (1997) 185:933-940]. As another example, GFAP directed expression of a human IL-1ra transgene attenuates edema, cytokine production and neurological deficits in a murine model of closed head injury [Tehranian, R., S. et al. J. Neurotrauma (2002) 19:939-951]. Finally, studies of penetrating brain injury in mice lacking the type 1 IL-1 receptor showed dramatic attenuation in microglial activation, leukocyte infiltration, and astrocyte activation [Basu, A., et al. J. Neurosci. (2002) 22:6071-6082]. Expression of numerous inflammatory mediators, including vascular cell adhesion molecule-1, several cytokines, and COX-2 was also greatly reduced in the IL-1R1 knockout mice, indicating that the IL-1 signaling pathway is essential for glial activation and the neuroinflammatory response. However, short-term infusion and viral delivery systems do not provide chronic stimuli and the genetic knockout systems are complicated by potential compensatory changes during development.

3. Nucleic Acids Related to Inflammation

Disclosed are a variety of nucleic acids which are related to inflammation. The nucleic acids can be used to produce transgenic cells or animals, for example, and they can be used in cell systems which are designed to produce or analyze the nucleic acids.

In certain embodiments the nucleic acids typically comprise a number of elements. Each of these elements is discussed below, and it is understood that at a fundamental level the elements can be defined functionally by what they do in combination with known or understood function for that type of element.

The disclosed nucleic acids can comprise an inactivating element, such as a stop sequence, which often can be flanked by recombination sites, such as flox sites, a positive transcription regulator sequence, such as a promoter and/or enhancer, a signal sequence, such as a sequence for trafficking of a the protein product(s) expressed from the nucleic acid, an inflammation element, which is typically an element encoding an inflammation sequence, a marker sequence, such as lacZ, typically an Intra Ribosome Expression Sequence (IRES), if multiple proteins will be expressed from the same construct, and a poly A tail. Alternatively, delivery of a ssIL-1β-IES-gfp gene can be delivered to the site of choice using a viral vector, such as the feline immunodeficiency virus vector system, adeno-associated viral system, etc. These elements are discussed herein. An example of a nucleic acid comprising these elements is shown in SEQ ID NO:70.

a) Inactivating Cassette

The inactivating cassette is a sequence which can prevent the transcription of one or more gene sequences contained within the nucleic acid. The inactivating cassette often can comprise a stop sequence, or transcriptional termination sequence, such as the open reading frame of a drug resistance gene that can be used as a selection marker, typically followed by the poly A tail sequence. In one example, it is a neomycin gene driven by the PGK promoter, followed by the bovine poly A tail, and this can be flanked by recombination sites, such as loxP sites (See SEQ ID NO:34).

As discussed herein, the inactivating cassette is often flanked by recombination sequences, such that in the presence of a cognate recombinase, the inactivating cassette is excised from the inflammation nucleic acid. Recombination sequences and their use are discussed herein.

b) Positive Transcription Regulator Cassette

A positive transcription regulator cassette is a cassette that is typically operably linked to the inflammatory cytokine or other proteins to be expressed, and which causes transcription of the operably linked sequence at either a basal, background, level or typically at a level above basal transcription levels. The positive transcription regulator often is a promoter or an enhancer, can be constitutively active, such as a CMV promoter, or conditionally active, such as a neural specific promoter, such as a neuronal enolase promoter (NSE) or a collagen or bone specific promoter, such as the COLL1A1 promoter (Example, SEQ ID NO:29) and the COLL2A1 (Example, SEQ ID NO:30), or astrocyte specific promoter, such as the glian fibrillary acidic protein promoter (GFAP).

(1) Promoter

The nucleic acids that are delivered to cells typically contain expression controlling systems, such as positive transcription regulators. However, the specific regulatory nucleotide sequence can be any sequence. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

Thus, the regulatory nucleotide sequence can comprise a promoter. Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus a CMV promoter, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell. Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell. Bio. 4: 1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, α-fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promoter and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases, for example). Other preferred promoters are SV40 promoters, cytomegalovirus (full length promoter), and retroviral vector LTF. In one aspect, the promoter of the provided composition is CMV (Example SEQ ID NO:26).

Various CMV and beta actin promoters are set forth in SEQ ID NOs:26-27, 49-69. Other human cytomegalovirus promoter regions can be found in accession numbers M64940, Human cytomegalovirus IE-1 promoter region, M64944 Human cytomegalovirus IE-1 promoter region, M64943 Human cytomegalovirus IE-1 promoter region, M64942 Human cytomegalovirus IE-1 promoter region, M64941 Human cytomegalovirus IE-1 promoter region (All of which are herein incorporated by reference at least for their sequence and information).

The promoter of the provided composition can be a cell-selective promoter. It has been shown that all specific regulatory elements can be cloned and used to construct expression vectors that are selectively expressed in specific cell types such as melanoma cells. The specific promoter used will therefore depend on the desired cell type to be targeted. For example, the glial fibrillary acetic protein (GFAP) promoter has been used to selectively express genes in cells of glial origin. The COLL1A1 promoter (Example SEQ ID NO:29) can be used to selectively express genes in chondrocytes, osteocytes, and fibroblasts, which can be found in joints. It is understood that all known and compatible selective promoters are considered in the provided composition. In one aspect, the promoter of the provided composition is GFAP (Example SEQ ID NO:28). In another aspect, the promoter is COLL1A2 promoter (Example SEQ ID NO:30).

c) Signal Sequence

In the provided composition, the nucleic acid can further comprises a nucleic acid encoding a peptide signal sequence (SS), such as a secretion signal sequence. In one aspect, the peptide secretion signal is derived from the IL-1 receptor antagonist (IL-1ra) gene. (Example SEQ ID NO:32).

d) Recombination Sequence

Provided herein are compositions and methods utilizing recombinase technology, such as Cre recombinase or Flp recombinase, wherein the composition comprises a recombination site, such as a loxP-flanked “floxed” nucleic acid sequence, for Cre recombinase. The properties and characteristics of Cre recombinase and flox sites are are exemplary of recombinases and recombination sites.

(1) LoxP

U.S. Pat. No. 4,959,317 and U.S. Pat. No. 5,434,066 are incorporated herein by reference for their teaching of the use of Cre recombinase in the site-specific recombination of DNA in eukaryotic cells. The term “Cre” recombinase, as used herein, refers to a protein having an activity that is substantially similar to the site-specific recombinase activity of the Cre protein of bacteriophage P1 (Hamilton, D. L., et al., J. Mol. Biol. 178:481-486 (1984), herein incorporated by reference for its teaching of Cre recombinase). The Cre protein of bacteriophage P1 mediates site-specific recombination between specialized sequences, known as “loxP” sequences. Hoess, R., et al., Proc. Natl. Acad. Sci. USA 79:3398-3402 (1982) and Sauer, B. L., U.S. Pat. No. 4,959,317 are herein incorporated by reference for their teaching of the lox sequences. The loxP site has been shown to consist of a double-stranded 34 bp sequence (SEQ ID NOS: 46 and 47):

SEQ ID NO:46

5′ ATAACTTCGTATAATGTATGCTATACGAAGTTAT 3′

SEQ ID NO:47

5′ ATAACTTCGTATAGCATACATTATACGAAGTTAT 3′

This sequence contains two 13 bp inverted repeat sequences which are separated from one another by an 8 bp spacer region. Other suitable lox sites include LoxB, LoxL and LoxR sites which are nucleotide sequences isolated from E. coli. These sequences are disclosed and described by Hoess et al., Proc. Natl. Acad. Sci. USA 79:3398-3402 (1982), herein incorporated by reference for the teaching of lox sites. Lox sites can also be produced by a variety of synthetic techniques which are known in the art. For example, synthetic techniques for producing lox sites are disclosed by Ito et al., Nuc. Acid Res., 10:1755 (1982) and Ogilvie et al., Science 214:270 (1981), the disclosures of which are incorporated herein by reference for their teaching of these synthetic techniques.

The Cre protein mediates recombination between two loxP sequences (Sternberg, N., et al., Cold Spring Harbor Symp. Quant. Biol. 45:297-309 (1981)). These sequences may be present on the same DNA molecule, or they may be present on different molecules. Because the internal spacer sequence of the loxP site is asymmetrical, two loxP sites can exhibit directionality relative to one another (Hoess, R. H., et al., Proc. Natl. Acad, Sci. 81:1026-1029 (1984)). Thus, when two sites on the same DNA molecule are in a directly repeated orientation, Cre will excise the DNA between the sites (Abremski, K., et al., Cell 32:1301-1311 (1983)). However, if the sites are inverted with respect to each other, the DNA between them is not excised after recombination but is simply inverted. Thus, a circular DNA molecule having two loxP sites in direct orientation will recombine to produce two smaller circles, whereas circular molecules having two loxP sites in an inverted orientation simply invert the DNA sequence flanked by the loxP sites.

e) Inflammation Element

One element of the disclosed nucleic acids is the inflammation element. The inflammation element comprises sequence which encodes a protein that affects inflammation, a mediator or inflammation, such as COX, such as COX-2, IL-1, such as IL-1β, or IL-1ra. It is understood that these variants of these proteins having activities of at least 30%, 40%, 50%, 60%, 70%, 80%, 85%, 90%, or 95% are disclosed and can also be used.

(1) IL-1/IL-1ra

In one aspect, the inflammation element includes interleukin-1 (IL-1). IL-1 is a potent immunomodulating cytokine that exists as two principal isoforms, IL-1α and IL-1β. These two molecules show significant divergence in sequence and have somewhat different roles with IL-1α generally thought to be involved in direct cell:cell communication, whereas IL-1β is secreted. Nevertheless, these two molecules act through the same membrane-associated receptor known as IL-1 receptor type 1 (IL-1R1) to promote a proinflammatory signaling cascade that includes the activation of NFκB and MAP kinases [Rothwell, N. J. and G. N. Luheshi. Trends Neurosci. (2000) 23:618-625]. At least two molecules have been identified that antagonize the effects of IL-1. IL-1 receptor antagonist (IL-1ra) competes for receptor binding, and IL-1 receptor type 2 (IL-1R2), which lacks an intracellular domain, is thought to serve as a decoy receptor [Rothwell, N. J. and G. N. Luheshi. Trends Neurosci. (2000) 23:618-625]. Expression of each of these molecules is regulated. The contribution of IL-1 to an inflammatory response therefore depends on the relative balance of expression between these family members [Arend, W. P. Cytokine & Growth Factor Rev. (2002) 13:323-340]. In one example, the mature form of IL-1β is attached to the secretion signal from IL-ra which is the same sequence as the secretion signal sequence of IL-1β. Thus, the nucleic acid of the provided composition can encode human IL-1 (Examples SEQ ID NO:31 and 44).

The mediator of inflammation provided herein can also be an IL-1 antagonist. Thus, in one aspect, the nucleic acid of the provided composition can encode IL-1ra (Example SEQ ID NO: 32).

(2) Cyclooxygenase COX

In one aspect, the inflammation element includes the enzyme cyclooxygenase (COX). Cyclooxygenase is the principal target of non-steroidal anti-inflammatory drugs (NSAIDs), which are a mainstay of treatment for many inflammatory conditions. Cyclooxygenase catalyzes the first step in the conversion of arachidonic acid to prostanoids, a group of potent lipid mediators acting in diverse physiological processes.

Cyclooxygenase is known to exist in two isoforms: COX-1, which in many tissues appears to be constitutively expressed and responsible for homeostatic production of prostanoids, and COX-2, which is often referred to as the “inducible” isoform since its expression is rapidly modulated in response to diverse stimuli such as growth factors, cytokines, and hormones [O'Banion M K, et al. (1991). J Biol Chem 266: 23261-7; O'Banion M K, et al. (1992). Proc Natl Acad Sci U.S.A. 89:4888-92]. The distinction between these two COX isoforms, the roles they play, and the actions of prostanoids have been previously reviewed [Vane J R, et al. (1998). Annu. Rev. Pharmocol. Toxicol. 38:97-120; Smith, W L, et al. (2000). Annu Rev Biochem 69:145-82]. Thus, the nucleic acid of the provided composition can encode COX-2 (Example SEQ ID NO:33).

f) IRES Element

The IRES element is an internal ribosomal entry sequence (integrated) which can be isolated from the encephalomyocarditis crius (ECMV). This element allows multiple genes to be expressed and correctly translated when the genes are on the same construct. IRES sequences are discussed in for example, U.S. Pat. No. 4,937,190, which is herein incorporated by reference at least for material related to IRES sequences and their use. The IRES sequence can be obtained from a number of sources including commercial sources, such as the pIRES expressing vector from Clonetech (Clontech, Palo Alto Calif. 94303-4230). The sequence of an IRES sequence is set forth in SEQ ID NO:48 (Example).

g) Markers

In the provided composition, the nucleic acid can comprise a marker sequence. This marker product is used to determine if the gene has been delivered to the cell and once delivered is being expressed. The specific marker which is employed is typically not critical. In one aspect, the marker sequence comprises the E. Coli lacZ gene encoding β-galactosidase (lacZ). In another aspect, the marker sequence comprises nucleic acids encoding a fluorochrome. The fluorochrome can comprise, for example, green fluorescent protein (GFP).

In some embodiments the marker may be a selectable marker. Examples of suitable selectable markers for mammalian cells are dihydrofolate reductase (DHFR), thymidine kinase, neomycin, neomycin analog G418, hygromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puromycin and the green fluorescent protein.

h) Poly A Sequences

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct. One example of a poly A tail is the poly region of Bovine Growth Hormone

i) Vectors

Provided herein is a composition comprising a vector, wherein the vector comprises any of the nucleic acids provided herein.

4. Nucleic Acids Related to Recombinases

Sustained IL-1β expression by collagen 1-producing cells, including fibroblasts, chondrocyte and osteocytes, is expected to result in a mouse model of TMJ arthrosis and dysfunction. IL1β^{XAT} regulation is controlled in a temporal (time) and spatial (location) fashion by the Cre/loxP molecular genetic method utilizing (1) a germline transmitted recombinational substrate (COLL1-IL1β^{XAT}) containing a dormant transcription unit and (2) somatic gene transfer of a viral vector that expresses Cre recombinase which “activates” the gene of interest.

The somatic gene transfer of the recombinase, such as Cre can be performed using any type of vector system producing the recombinase. However, in certain embodiments, the vector system is a self inactivating vector system, wherein the promoter, for example, of the recombinase is flanked by recombination sites so that upon production of the recombinase, the recombinase will down regulate its own production. The delivery vectors for the recombinase can be CRE mediated.

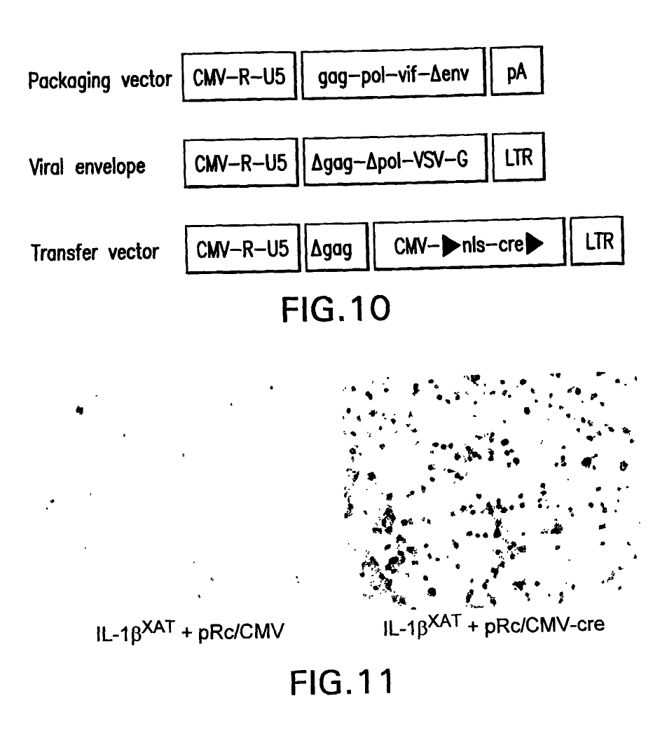

For example, activation of the dormant COLL1-IL1β^{XAT} can be mediated by the transfer of Cre recombinase to the area of interest (e.g. TMJ) via a self-inactivating Cre feline immunodeficiency virus FIV(Cre). The effects of this FIV vector system have been previously examined using the reporter gene lacZ (β-galactosidase) in mice that received intra-articular injections of a viral solution [Kyrkanides S, et al. (2004). J Dental Res 83: 65-70], wherein transduction of soft (articular disc) and hard (cartilage) TMJ tissues was demonstrated. The FIV(Cre)vector has been constructed by cloning a loxP-flanked (“floxed”) nlsCre cassette in the place of the lacZ gene; the nuclear localization signal (nls) was fused to the cre open reading frame by PCR and subsequently cloned into the TOPO 2.1 vector (Invitrogen) per manufacturer's instructions employing a custom-made floxed cloning cassette. The reason for developing a self-inactivating cre gene is based on a recent paper [Pfeifer A and Brandon E P, Kootstra Neeltje, Gage F H, Verma I M (2001). Proc Natl Acad Sci U.S.A. 98: 11450-5], whereby the authors reported cytotoxicity due to prolonged expression of Cre recombinase mediated by infection using a lentiviral vector. In the provided construct, upon production of adequate levels of Cre recombinase to produce excisional activation of COLL1-IL1β^{XAT} following successful transduction of target cells with FIV(Cre), Cre is anticipated to de-activate the cre gene by loxP-directed self excisional recombination. This strategy is anticipated to result in activation of COLL1-IL1β^{XAT} by FIV(Cre) avoiding any cytotoxic effects from Cre. Please see FIG. 10.

5. Nucleic Acids Properties and Primers and Probes

Provided herein is a composition comprising a nucleic acid sequence encoding an inflammatory mediator operably linked to a regulatory sequence via a loxP-flanked (floxed) inactivating cassette.

There are a variety of molecules disclosed herein that are nucleic acid based, including for example the nucleic acids that encode, for example IL-1β (Examples SEQ ID NO:31 and 44), or any of the nucleic acids disclosed herein for making the disclosed transgenics and models, or fragments thereof, as well as various functional nucleic acids. The disclosed nucleic acids are made up of, for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantageous that the antisense molecule be made up of nucleotide analogs that reduce the degradation of the antisense molecule in the cellular environment.

a) Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosine-1-yl (C), guanine-9-yl (G), uracil-1-yl (U), and thymine-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate). There are many varieties of these types of molecules available in the art and available herein.

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties. There are many varieties of these types of molecules available in the art and available herein.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid. There are many varieties of these types of molecules available in the art and available herein.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989, 86, 6553-6556). There are many varieties of these types of molecules available in the art and available herein.

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

b) Sequences

There are a variety of sequences related to the protein molecules involved in the signaling pathways disclosed herein, for example SEQ ID NO:31 and 44, or any of the nucleic acids disclosed herein for making IL-1β, all of which are encoded by nucleic acids or are nucleic acids. The sequences for the human analogs of these genes, as well as other analogs, and alleles of these genes, and splice variants and other types of variants, are available in a variety of protein and gene databases, including Genbank. Those sequences available at the time of filing this application at Genbank are herein incorporated by reference in their entireties as well as for individual subsequences contained therein. Genbank can be accessed at http://www.ncbi.nih.gov/entrez/query.fcgi. Those of skill in the art understand how to resolve sequence discrepancies and differences and to adjust the compositions and methods relating to a particular sequence to other related sequences. Primers and/or probes can be designed for any given sequence given the information disclosed herein and known in the art.

c) Primers and Probes

Disclosed are compositions including primers and probes, which are capable of interacting with the disclosed nucleic acids, such as IL-1β, as disclosed herein. In certain embodiments the primers are used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with the disclosed nucleic acids or region of the nucleic acids or they hybridize with the complement of the nucleic acids or complement of a region of the nucleic acids.

The size of the primers or probes for interaction with the nucleic acids in certain embodiments can be any size that supports the desired enzymatic manipulation of the primer, such as DNA amplification or the simple hybridization of the probe or primer. A typical primer or probe would be at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In other embodiments a primer or probe can be less than or equal to 6, 7, 8, 9, 10, 11, 12 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

The primers for the IL-1β gene typically will be used to produce an amplified DNA product that contains a region of the IL-1β gene or the complete gene. In general, typically the size of the product will be such that the size can be accurately determined to within 3, or 2 or 1 nucleotides.

In certain embodiments this product is at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In other embodiments the product is less than or equal to 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

d) Sequence Similarities

It is understood that as discussed herein the use of the terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

In general, it is understood that one way to define any known variants and derivatives or those that might arise, of the disclosed genes and proteins herein, is through defining the variants and derivatives in terms of homology to specific known sequences. This identity of particular sequences disclosed herein is also discussed elsewhere herein. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity, and be disclosed herein.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

e) Hybridization

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization may involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987:154:367, 1987 which is herein incorporated by reference for material at least related to hybridization of nucleic acids). A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

6. Peptides

a) Protein Variants

As discussed herein there are numerous variants of the proteins, such as the IL-1β protein that are known and herein contemplated. In addition, to the known functional strain variants there are derivatives of the disclosed proteins which also function in the disclosed methods and compositions. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 1
Amino Acid Abbreviations

| | |
|---|---|
| Amino Acid | Abbreviations |

| | | |
|---|---|---|
| alanine | Ala | A |
| allosoleucine | AIle | |
| arginine | Arg | R |
| asparagine | Asn | N |
| aspartic acid | Asp | D |
| cysteine | Cys | C |
| glutamic acid | Glu | E |
| glutamine | Gln | Q |
| glycine | Gly | G |
| histidine | His | H |
| isolelucine | Ile | I |
| leucine | Leu | L |
| lysine | Lys | K |
| phenylalanine | Phe | F |
| proline | Pro | P |
| pyroglutamic acid | pGlu | |
| serine | Ser | S |
| threonine | Thr | T |
| tyrosine | Tyr | Y |
| tryptophan | Trp | W |
| valine | Val | V |

TABLE 2
Amino Acid Substitutions

| Original | Exemplary Conservative Substitutions |
|---|---|
| Residue | others are known in the art. |
| Ala | Ser |
| Arg | Lys; Gln |
| Asn | Gln; His |
| Asp | Glu |
| Cys | Ser |
| Gln | Asn, Lys |
| Glu | Asp |
| Gly | Pro |
| His | Asn; Gln |
| Ile | Leu; Val |
| Leu | Ile; Val |
| Lys | Arg; Gln |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and aspartyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. For example, SEQ ID NO:31 sets forth a particular sequence of IL-1β and SEQ ID NO:32 sets forth a particular sequence of a IL-1ra encoding their respective proteins. Specifically disclosed are variants of these and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. It is also understood that while no amino acid sequence indicates what particular DNA sequence encodes that protein within an organism, where particular variants of a disclosed protein are disclosed herein, the known nucleic acid sequence that encodes that protein in the particular species from which that protein arises is also known and herein disclosed and described.

It is understood that there are numerous amino acid and peptide analogs which can be incorporated into the disclosed compositions. For example, there are numerous D amino acids or amino acids which have a different functional substituent then the amino acids shown in Table 1 and Table 2. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way (Thorson et al., Methods in Molec. Biol. 77:43-73 (1991), Zoller, Current Opinion in Biotechnology, 3:348-354 (1992); Ibba, Biotechnology & Genetic Engineering Reviews 13:197-216 (1995), Cahill et al., TIBS, 14(10):400-403 (1989); Benner, TIB Tech, 12:158-163 (1994); Ibba and Hennecke, Bio/technology, 12:678-682 (1994) all of which are herein incorporated by reference at least for material related to amino acid analogs).

Molecules can be produced that resemble peptides, but which are not connected via a natural peptide linkage. For example, linkages for amino acids or amino acid analogs can include CH₂NH—, —CH₂S—, —CH₂—CH₂—, —CH═CH—(cis and trans), —COCH₂—, —CH(OH)CH₂—, and —CHH₂SO—(These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CH H₂—S); Hann J. Chem. Soc Perkin Trans. I 307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations. (Rizo and Gierasch Ann. Rev. Biochem. 61:387 (1992), incorporated herein by reference).

7. Cells

It is understood that prokaryotic and/or eukaryotic cells may be used in the creation, propagation, or delivery of the provided nucleic acids and vectors. The specific selection of the cell used is typically not important and is typically driven by the end goal for the cell. Therefore, provided herein is a composition comprising a cell, wherein the cell comprises any one of the vectors or nucleic acids or proteins provided herein. Examples of cells include primary mouse or rat or human fibroblasts, primary mouse or rat or human chondrocytes, NIH 3T3 fibroblast cell line, and ATDC5 chondrocyte cell line.

8. Animals

Provided herein are transgenic animals comprising germline transmission of any of the vectors or nucleic acids provided herein. In one aspect, the transgenic animal provided herein is an excision activated transgenic (XAT) animal. The disclosed transgenic animals can have have temporally and spatially regulated transgene expression (Brooks, A I, et al. 1991. Nature Biotech 15:57-62; Brooks, A I, et al. 1999. Neuroreport 10:337-344; Brooks, A I., et al. 2000. Proc Natl Acad Sci USA 97:13378-13383) of an inflammation element. It is understood that where the transgenic animal comprises a nucleic acid comprising a recombination site, as disclosed herein, delivery of a recombinase, such as Cre recombinase to cells within the provided transgenic animal will result in the expression of the inflammatory modulator, e.g., IL-1β, IL-1ra, COX-2, within those cells.

By a “transgene” is meant a nucleic acid sequence that is inserted by artifice into a cell and becomes a part of the genome of that cell and its progeny. Such a transgene may be (but is not necessarily) partly or entirely heterologous (e.g., derived from a different species) to the cell. The term “transgene” broadly refers to any nucleic acid that is introduced into an animal's genome, including but not limited to genes or DNA having sequences which are perhaps not normally present in the genome, genes which are present, but not normally transcribed and translated (“expressed”) in a given genome, or any other gene or DNA which one desires to introduce into the genome. This may include genes which may normally be present in the nontransgenic genome but which one desires to have altered in expression, or which one desires to introduce in an altered or variant form. A transgene can include one or more transcriptional regulatory sequences and any other nucleic acid, such as introns, that may be necessary for optimal expression of a selected nucleic acid. A transgene can be as few as a couple of nucleotides long, but is preferably at least about 50, 100, 150, 200, 250, 300, 350, 400, or 500 nucleotides long or even longer and can be, e.g., an entire genome. A transgene can be coding or non-coding sequences, or a combination thereof. A transgene usually comprises a regulatory element that is capable of driving the expression of one or more transgenes under appropriate conditions. By “transgenic animal” is meant an animal comprising a transgene as described above. Transgenic animals are made by techniques that are well known in the art. The disclosed nucleic acids, in whole or in part, in any combination, can be transgenes as disclosed herein.

Disclosed are animals produced by the process of transfecting a cell within the animal with any of the nucleic acid molecules disclosed herein. Disclosed are animals produced by the process of transfecting a cell within the animal any of the nucleic acid molecules disclosed herein, wherein the animal is a mammal. Also disclosed are animals produced by the process of transfecting a cell within the animal any of the nucleic acid molecules disclosed herein, wherein the mammal is mouse, rat, rabbit, cow, sheep, pig, or primate.

The disclosed transgenic animals can be any non-human animal, preferably a non-human mammal (e.g. mouse, rat, rabbit, squirrel, hamster, rabbits, guinea pigs, pigs, micro-pigs, prairie dogs, baboons, squirrel monkeys and chimpanzees, etc), bird or an amphibian, in which one or more cells contain heterologous nucleic acid introduced by way of human intervention, such as by transgenic techniques well known in the art. The nucleic acid is introduced into the cell, directly or indirectly, by introduction into a precursor of the cell, such as by microinjection or by infection with a recombinant virus. The disclosed transgenic animals can also include the progeny of animals which had been directly manipulated or which were the original animal to receive one or more of the disclosed nucleic acids. This molecule may be integrated within a chromosome, or it may be extrachromosomally replicating DNA. For techniques related to the production of transgenic animals, see, inter alia, Hogan et al (1986) Manipulating the Mouse Embryo-A Laboratory Manual Cold Spring Harbor Laboratory, Cold Spring Harbor, N.Y., 1986).

Animals suitable for transgenic experiments can be obtained from standard commercial sources such as Charles River (Wilmington, Mass.), Taconic (Germantown, N.Y.), and Harlan Sprague Dawley (Indianapolis, Ind.). For example, if the transgenic animal is a mouse, many mouse strains are suitable, but C57BL/6 female mice can be used for embryo retrieval and transfer. C57BL/6 males can be used for mating and vasectomized C57BL/6 studs can be used to stimulate pseudopregnancy. Vasectomized mice and rats can be obtained from the supplier. Transgenic animals can be made by any known procedure, including microinjection methods, and embryonic stem cells methods. The procedures for manipulation of the rodent embryo and for microinjection of DNA are described in detail in Hogan et al., Manipulating the Mouse Embryo (Cold Spring Harbor Laboratory, Cold Spring Harbor, N.Y., 1986), the teachings of which are generally known and are incorporated herein.

Transgenic animals can be identified by analyzing their DNA. For this purpose, for example, when the transgenic animal is an animal with a tail, such as rodent, tail samples (1 to 2 cm) can be removed from three week old animals. DNA from these or other samples can then be prepared and analyzed, for example, by Southern blot, PCR, or slot blot to detect transgenic founder (F (0)) animals and their progeny (F (1) and F (2)). The present invention further provides transgenic non-human animals that are progeny of crosses between a transgenic animal of the invention and a second animal. Transgenic animals can be bred with other transgenic animals, where the two transgenic animals were generated using different transgenes, to test the effect of one gene product on another gene product or to test the combined effects of two gene products.

a) Somatic Mosaic Technology

Somatic mosaic technology for creating transgenic animals with temporally and spatially regulated transgene expression was developed and first described in Howard Federoff's laboratory [Brooks, A. I., et al. Nature Biotech. (1997) 15:57-62; Brooks, A. I., et al. NeuroReport (1999) 10:337-344; and Brooks, A. I., et al. Proc. Natl. Acad. Sci. USA (2000) 97:13378-13383]. In somatic mosaic technology a transgene is expressed in either a temporally regulated way or in a spatially regulated way or the gene can be regulated in both ways. The original work involved development of a nerve growth factor (NGF) XAT mouse line and the use of HSV amplicon vectors carrying cre recombinase to induce hippocampal expression of NGF. Animals undergoing such treatment showed elevated levels of NGF (10-fold) [Brooks, A. I., et al. Nature Biotech. (1997) 15:57-62] and histological evidence of increased cholinergic projection to the specific region of hippocampus expressing the transgene [Brooks, A. I., et al. NeuroReport (1999) 10:337-344]. Moreover, NGF-activated animals showed enhanced learning and evidence for behavioral modulation of the septohippocampal pathways [Brooks, A. I., et al. Proc. Natl. Acad. Sci. USA (2000) 97:13378-13383]. Provided herein is the use this technology to generate transgenic mice that can be manipulated to regionally and temporally express hIL-1β or its antagonist.

(1) Development of the IL-1β Somatic Mosaic Mouse

One disclosed embodiment are IL-1β somatic mosaic mice, which are disclosed such that the IL-1β can be constitutively produced, or conditionally expressed in selective tissues, such as bone related, such as chondrocytes, or neural related cells, or temporally expressed. Somatic mosaic analysis is a molecular genetic method that allows one to induce long-term expression of a gene of interest, due to a permanent change in the genetic constitution of infected cells, at a particular location (i.e. TMJ) and during a specific developmental stage. The somatic mosaic analysis model offers significant advantages compared to traditional transgenic mice, because it avoids compensatory adaptations often encountered in transgenic mice during development and allows regional activation of a gene [Brooks A I, et al. (1997). Nat Biotech 15(1):57-62; Brooks A I, et al. (1999). Neuroreport 10:337-44; and Maguire-Zeiss K A, et al. (2002). Neurobiol Aging 23:977-84].

The construction and regulation of COLL1-IL1β^{XAT} by cre recombinase in vitro is discussed in the Examples. COLL1-IL1β^{XAT} transgenic mice construction and development are discussed in the examples. Typically, each set of microinjections yields 15-25 pups, of which 20-25% harbor the transgene of interest [Polites H G and Pinkert C A (2002). DNA microinjection and transgenic animal production, p. 15-70. In C. A. Pinkert (ed.), Transgenic animal technology: a laboratory handbook. 2nd ed. Academic Press, Inc., San Diego]. Genotyping is usually performed at weaning, and breeding of transgenic founders to establish lines generally commences around 6-8 weeks of age [Overbeek P A (2002). DNA microinjection and transgenic animal production, p. 72-112. In C. A. Pinkert (Ed.), Transgenic animal technology: a laboratory handbook. 2nd ed. Academic Press, Inc., San Diego; Tinkle B T and Jay G (2002). Analysis of transgene integration, p. 459-474. In C A Pinkert (ed.), Transgenic animal technology: a laboratory handbook. 2nd ed. Academic Press, Inc., San Diego.]. The presence and number of transgene copies in these founders can be determined by methods routinely employed, such as conventional or quantitative PCR on tail DNA extracts using primers specifically designed for the COLL1-IL1β^{XAT} Tg, then confirmed by Southern blot analysis using whole length probes) [Tinkle B T and Jay G (2002). Analysis of transgene integration, p. 459-474. In C A Pinkert (ed.), Transgenic animal technology: a laboratory handbook. 2nd ed. Academic Press, Inc., San Diego; Irwin, M. H., et al. (2002). PCR optimization for detection of transgene integration, p. 475-484. In C. A. Pinkert (ed.), Transgenic animal technology: a laboratory handbook. 2nd ed. Academic Press, Inc., San Diego; and Nagy A, et al. (2003) Manipulating the Mouse Embryo: A Laboratory Manual. 3rd ed., Cold Spring Harbor Laboratory Press, Cold Spring Harbor, N.Y.]. It is anticipated that three to five transgenic founders can be identified in each set of microinjections; these mice can be bred to C57BL/6 stock mice for analysis of germ-line transmission of a functional transgene [Ngo L and Jay G (2002). Analysis of transgene expression, p. 486-513. In C A Pinkert (ed.), Transgenic animal technology: a laboratory handbook. 2nd ed. Academic Press, Inc., San Diego and Pinkert C A (2003). Transgenic animal technology: Alternatives in genotyping and phenotyping. Comp Med 53:116-29]. Moreover, a second series of microinjections can be performed if an adequate number of germ-line competent, transgene-expressing founders are not produced by this initial experiment. It is anticipated that the transgene can be maintained in a heterozygous state on the C57BL/6 background.

9. Kits

Disclosed herein are kits that are drawn to reagents that can be used in practicing the methods disclosed herein. The kits can include any reagent or combination of reagentS discussed herein or that would be understood to be required or beneficial in the practice of the disclosed methods. For example, the kits could include primers to perform the amplification reactions discussed in certain embodiments of the methods, as well as the buffers and enzymes required to use the primers as intended. For example, disclosed is a kit for screening compounds that affect inflammatory disease, comprising the provided XAT animal and an expression vector for delivery of Cre recombinase to desired target in the animal, e.g. FIVcre.

### C. METHODS OF MAKING THE COMPOSITIONS

The compositions disclosed herein and the compositions necessary to perform the disclosed methods can be made using any method known to those of skill in the art for that particular reagent or compound unless otherwise specifically noted.

Nucleic Acid Synthesis

For example, the nucleic acids, such as, the oligonucleotides to be used as primers can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System 1 Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

2. Cells

Provided is a composition comprising a cell, wherein the cell comprises any one of the nucleic acids or vectors provided herein.

a) Delivery of the Compositions to Cells

There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

(1) Nucleic Acid Based Delivery Systems

Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)).

As used herein, plasmid or viral vectors are agents that transport the disclosed nucleic acids, such as the nucleic acids encoding an inflammation molecule into the cell without degradation and include a promoter yielding expression of the gene in the cells into which it is delivered. In some embodiments the vectors are derived from either a virus or a retrovirus. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors can have higher transaction (ability to introduce genes) abilities than chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

(a) Retroviral Vectors

A retrovirus is an animal virus belonging to the virus family of Retroviridae, including any types, subfamilies, genus, or tropisms. Retroviral vectors, in general, are described by Verma, I. M., Retroviral vectors for gene transfer. In Microbiology-1985, American Society for Microbiology, pp. 229-232, Washington, (1985), which is incorporated by reference herein. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

(b) Adenoviral Vectors

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virons are generated in a cell line such as the human 293 cell line. In another preferred embodiment both the E1 and E3 genes are removed from the adenovirus genome.

(c) Adeno-Associated Viral Vectors

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus.

Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference for material related to the AAV vector.

The disclosed vectors thus provide DNA molecules which are capable of integration into a mammalian chromosome without substantial toxicity.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

(d) Leutiviral Vectors

The vectors can be lentiviral vectors, including but not limited to, SIV vectors, HIV vectors or a hybrid construct of these vectors, including viruses with the HIV backbone. These vectors also include first, second and third generation lentiviruses. Third generation lentiviruses have lentiviral packaging genes split into at least 3 independent plasmids or constructs. Also vectors can be any viral family that share the properties of these viruses which make them suitable for use as vectors. Lentiviral vectors are a special type of retroviral vector which are typically characterized by having a long incubation period for infection. Furthermore, lentiviral vectors can infect non-dividing cells. Lentiviral vectors are based on the nucleic acid backbone of a virus from the lentiviral family of viruses. Typically, a lentiviral vector contains the 5′ and 3′ LTR regions of a lentivirus, such as SIV and HIV. Lentiviral vectors also typically contain the Rev Responsive Element (RRE) of a lentivirus, such as SIV and HIV.

(i) Felite Immunodeficiency Viral Vectors

One type of vector that the disclosed constructs can be delivered in is the VSV-G pseudotyped Feline Immunodeficiency Virus system developed by Poeschla et al. Nature Med. (1998) 4:354-357 (Incorporated by reference herein at least for material related to FIV vectors and their use). This lentivirus has been shown to efficiently infect dividing, growth arrested as well as post-mitotic cells. Furthermore, due to its lentiviral properties, it allows for incorporation of the transgene into the host's genome, leading to stable gene expression. This is a 3-vector system, whereby each confers distinct instructions: the FIV vector carries the transgene of interest and lentiviral apparatus with mutated packaging and envelope genes. A vesicular stomatitis virus G-glycoprotein vector (VSV-G; Burns et al., Proc. Natl. Acad. Sci. USA 90:8033-8037. 1993) contributes to the formation of the viral envelope in trans. The third vector confers packaging instructions in trans (Poeschla et al. Nature Med. (1998) 4:354-357). FIV production is accomplished in vitro following co-transfection of the aforementioned vectors into 293-T cells. The FIV-rich supernatant is then collected, filtered and can be used directly or following concentration by centrifugation. Titers routinely range between 10⁴-10⁷ bfu/ml.

(e) Packaging Vectors

As discussed above, retroviral vectors are based on retroviruses which contain a number of different sequence elements that control things as diverse as integration of the virus, replication of the integrated virus, replication of un-integrated virus, cellular invasion, and packaging of the virus into infectious particles. While the vectors in theory could contain all of their necessary elements, as well as an exogenous gene element (if the exogenous gene element is small enough) typically many of the necessary elements are removed. Since all of the packaging and replication components have been removed from the typical retroviral, including lentiviral, vectors which will be used within a subject, the vectors need to be packaged into the initial infectious particle through the use of packaging vectors and packaging cell lines. Typically retroviral vectors have been engineered so that the myriad functions of the retrovirus are separated onto at least two vectors, a packaging vector and a delivery vector. This type of system then requires the presence of all of the vectors providing all of the elements in the same cell before an infectious particle can be produced. The packaging vector typically carries the structural and replication genes derived from the retrovirus, and the delivery vector is the vector that carries the exogenous gene element that is preferably expressed in the target cell. These types of systems can split the packaging functions of the packaging vector into multiple vectors, e.g., third-generation lentivirus systems. Dull, T. et al., “A Third-generation lentivirus vector with a conditional packaging system” J. Virol 72(11):8463-71 (1998)

Retroviruses typically contain an envelope protein (env). The Env protein is in essence the protein which surrounds the nucleic acid cargo. Furthermore cellular infection specificity is based on the particular Env protein associated with a typical retrovirus. In typical packaging vector/delivery vector systems, the Env protein is expressed from a separate vector than for example the protease (pro) or integrase (in) proteins.

(f) Packaging Cell Lines

The vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals. One type of packaging cell line is a 293 cell line.

(g) Large Payload Viral Vectors

Molecular genetic experiments with large human herpes viruses have provided a means whereby large heterologous DNA fragments can be cloned, propagated and established in cells permissive for infection with herpes viruses (Sun et al., Nature genetics 8: 33-41, 1994; Cotter and Robertson, Curr Opin Mol Ther 5: 633-644, 1999). These large DNA viruses (herpes simplex virus (HSV) and Epstein-Barr virus (EBV), have the potential to deliver fragments of human heterologous DNA>150 kb to specific cells. EBV recombinants can maintain large pieces of DNA in the infected B-cells as episomal DNA. Individual clones carried human genomic inserts up to 330 kb appeared genetically stable The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA >220 kb and to infect cells that can stably maintain DNA as episomes.

Other useful systems include, for example, replicating and host-restricted non-replicating vaccinia virus vectors.

(2) Non-Nucleic Acid Based Systems

The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed nucleic acids or vectors for example, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J. Resp. Cell. Mol. Biol. 1:95-100 (1989); Felgner et al. Proc. Natl. Acad. Sci. USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the disclosed nucleic acid or vector can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). These techniques can be used for a variety of other specific cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

Nucleic acids that are delivered to cells which are to be integrated into the host cell genome, typically contain integration sequences. These sequences are often viral related sequences, particularly when viral based systems are used. These viral intergration systems can also be incorporated into nucleic acids which are to be delivered using a non-nucleic acid based system of deliver, such as a liposome, so that the nucleic acid contained in the delivery system can be come integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

(3) In Vivo/Ex Vivo

As described above, the compositions can be administered in a pharmaceutically acceptable carrier and can be delivered to the subject's cells ill vivo and/or ex vivo by a variety of mechanisms well known in the art (e.g., uptake of naked DNA, liposome fusion, intramuscular injection of DNA via a gene gun, endocytosis and the like).

If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

3. Transgenic Mice Models

a) Methods of Producing Transgenic Animals

The nucleic acids and vectors provided herein can be used to produce excision transgenic, or XAT, animals. Various methods are known for producing a transgenic animal. In one method, an embryo at the pronuclear stage (a “one cell embryo”) is harvested from a female and the transgene is microinjected into the embryo, in which case the transgene will be chromosomally integrated into the germ cells and somatic cells of the resulting mature animal. In another method, embryonic stem cells are isolated and the transgene is incorporated into the stem cells by electroporation, plasmid transfection or microinjection; the stem cells are then reintroduced into the embryo, where they colonize and contribute to the germ line. Methods for microinjection of polynucleotides into mammalian species are described, for example, in U.S. Pat. No. 4,873,191, which is incorporated herein by reference. In yet another method, embryonic cells are infected with a retrovirus containing the transgene, whereby the germ cells of the embryo have the transgene chromosomally integrated therein. When the animals to be made transgenic are avian, microinjection into the pronucleus of the fertilized egg is problematic because avian fertilized ova generally go through cell division for the first twenty hours in the oviduct and, therefore, the pronucleus is inaccessible. Thus, the retrovirus infection method is preferred for making transgenic avian species (see U.S. Pat. No. 5,162,215, which is incorporated herein by reference). If microinjection is to be used with avian species, however, the embryo can be obtained from a sacrificed when approximately 2.5 hours after the laying of the previous laid egg, the transgene is microinjected into the cytoplasm of the germinal disc and the embryo is cultured in a host shell until maturity (Love et al., Biotechnology 12, 1994). When the animals to be made transgenic are bovine or porcine, microinjection can be hampered by the opacity of the ova, thereby making the nuclei difficult to identify by traditional differential interference-contrast microscopy. To overcome this problem, the ova first can be centrifuged to segregate the pronuclei for better visualization.

The transgene can be introduced into embryonal target cells at various developmental stages, and different methods are selected depending on the stage of development of the embryonal target cell. The zygote is the best target for microinjection. The use of zygotes as a target for gene transfer has a major advantage in that the injected DNA can incorporate into the host gene before the first cleavage (Brinster et al., Proc. Natl. Acad. Sci., USA 82:4438-4442, 1985). As a consequence, all cells of the transgenic non-human animal carry the incorporated transgene, thus contributing to efficient transmission of the transgene to offspring of the founder, since 50% of the germ cells will harbor the transgene.

A transgenic animal can be produced by crossbreeding two chimeric animals, each of which includes exogenous genetic material within cells used in reproduction. Twenty-five percent of the resulting offspring will be transgenic animals that are homozygous for the exogenous genetic material, 50% of the resulting animals will be heterozygous, and the remaining 25% will lack the exogenous genetic material and have a wild type phenotype.

In the microinjection method, the transgene is digested and purified free from any vector DNA, for example, by gel electrophoresis. The transgene can include an operatively associated promoter, which interacts with cellular proteins involved in transcription, and provides for constitutive expression, tissue specific expression, developmental stage specific expression, or the like. Such promoters include those from cytomegalovirus (CMV), Moloney leukemia virus (MLV), and herpes virus, as well as those from the genes encoding metallothionein, skeletal actin, Phosphenolpyruvate carboxylase (PEPCK), phosphoglycerate (PGK), dihydrofolate reductase (DHFR), and thymidine kinase (TK). Promoters from viral long terminal repeats (LTRs) such as Rous sarcoma virus LTR also can be employed. When the animals to be made transgenic are avian, preferred promoters include those for the chicken [bgr]-globin gene, chicken lysozyme gene, and avian leukosis virus. Constructs useful in plasmid transfection of embryonic stem cells will employ additional regulatory elements, including, for example, enhancer elements to stimulate transcription, splice acceptors, termination and polyadenylation signals, ribosome binding sites to permit translation, and the like.

In the retroviral infection method, the developing non-human embryo can be cultured in vitro to the blastocyst stage. During this time, the blastomeres can be targets for retroviral infection (Jaenich, Proc. Natl. Acad. Sci. USA 73:1260-1264, 1976). Efficient infection of the blastomeres is obtained by enzymatic treatment to remove the zona pellucida (Hogan et al., Manipulating the Mouse Embryo (Cold Spring Harbor Laboratory Press, 1986). The viral vector system used to introduce the transgene is typically a replication-defective retrovirus carrying the transgene (Jahner et al., Proc. Natl. Acad. Sci., USA 82:6927-6931, 1985; Van der Putten et al., Proc. Natl. Acad. Sci. USA 82:6148-6152, 1985). Transfection is easily and efficiently obtained by culturing the blastomeres on a monolayer of virus producing cells (Van der Putten et al., supra, 1985; Stewart et al., EMBO J. 6:383-388, 1987). Alternatively, infection can be performed at a later stage. Virus or virus-producing cells can be injected into the blastocoele (Jahner et al., Nature 298:623-628, 1982). Most of the founders will be mosaic for the transgene since incorporation occurs only in a subset of the cells which formed the transgenic nonhuman animal. Further, the founder can contain various retroviral insertions of the transgene at different positions in the genome, which generally will segregate in the offspring. In addition, it is also possible to introduce transgenes into the germ line, albeit with low efficiency, by intrauterine retroviral infection of the mid-gestation embryo (Jahner et al., supra, 1982).

Embryonal stem cell (ES) also can be targeted for introduction of the transgene. ES cells are obtained from pre-implantation embryos cultured in vitro and fused with embryos (Evans et al. Nature 292:154-156, 1981; Bradley et al., Nature 309:255-258, 1984; Gossler et al., Proc. Natl. Acad. Sci., USA 83:9065-9069, 1986; Robertson et al., Nature 322:445-448, 1986). Transgenes can be efficiently introduced into the ES cells by DNA transfection or by retrovirus mediated transduction. Such transformed ES cells can thereafter be combined with blastocysts from a nonhuman animal. The ES cells thereafter colonize the embryo and contribute to the germ line of the resulting chimeric animal (see Jaenisch, Science 240:1468-1474, 1988).

Founder” generally refers to a first transgenic animal, which has been obtained from any of a variety of methods, e.g., pronuclei injection.

An “inbred animal line” is intended to refer to animals which are genetically identical at all endogenous loci.

b) Crosses

It is understood that the animals provided herein can be crossed with other animals. For example, wherein the provided animals are mice, they can be crossed with Alzheimer's Mice to study the effects of inflammatory mediators, e.g. IL-1β, on Alzheimer's disease. The association between Aβ deposition and inflammatory changes is reinforced by studies of transgenic mice harboring familial AD mutant genes. In transgenic mice expressing the Swedish APP mutation (Tg2576, APP_{K670N,M671L}; hereafter referred to as APPsw), microglial activation is intimately related to amyloid plaque deposition, with measures of both microglial size and activated microglial density being highest in the immediate vicinity of Aβ deposits [Frautschy, S. A, et al. Am. J. Pathol. (1998) 152:307-317]. These mice accumulate Aβ deposits over a protracted period of time, with plaques and glial changes becoming prominent after one year of age [Hsiao, K., P. Chapman, S, Nilsen, C. Eckman, Y. Harigaya, S. Younkin, F. Yang and G. Cole. Science (1996) 274:99-102]. Although other AD mouse models are available, the APPsw mice have been extensively characterized and offer an excellent resource for investigating mechanisms involved in Aβ deposition or Aβ induced inflammatory changes.

Examples of other transgenic animals for which it would be advantageous to cross with the provided transgenic animals include, but are not limited to, COX Null Mice, 3×Tg mice for Alzheimers disease [Oddo S, et al. Neuron. 2003 Jul. 31; 39(3):409-21], COL1a1-Cre mice.

4. Processes for Making the Compositions

Disclosed are processes for making the compositions as well as making the intermediates leading to the compositions. There are a variety of methods that can be used for making these compositions, such as synthetic chemical methods and standard molecular biology methods. It is understood that the methods of making these and the other disclosed compositions are specifically disclosed.

Disclosed are nucleic acid molecules produced by the process comprising linking in an operative way a nucleic acid comprising the sequence set forth herein and a sequence controlling the expression of the nucleic acid.

Also disclosed are nucleic acid molecules produced by the process comprising linking in an operative way a nucleic acid molecule comprising a sequence having 80% identity to a sequence set forth herein, and a sequence controlling the expression of the nucleic acid.

Disclosed are nucleic acid molecules produced by the process comprising linking in an operative way a nucleic acid molecule comprising a sequence that hybridizes under stringent hybridization conditions to a sequence set forth herein and a sequence controlling the expression of the nucleic acid.

Disclosed are nucleic acid molecules produced by the process comprising linking in an operative way a nucleic acid molecule comprising a sequence encoding a peptide set forth herein and a sequence controlling an expression of the nucleic acid molecule.

Disclosed are nucleic acid molecules produced by the process comprising linking in an operative way a nucleic acid molecule comprising a sequence encoding a peptide having 80% identity to a peptide set forth herein and a sequence controlling an expression of the nucleic acid molecule.

Disclosed are nucleic acids produced by the process comprising linking in an operative way a nucleic acid molecule comprising a sequence encoding a peptide having 80% identity to a peptide set forth herein wherein any change from the sequences set forth herein are conservative changes and a sequence controlling an expression of the nucleic acid molecule.

Disclosed are cells produced by the process of transforming the cell with any of the disclosed nucleic acids. Disclosed are cells produced by the process of transforming the cell with any of the non-naturally occurring disclosed nucleic acids.

Disclosed are any of the disclosed peptides produced by the process of expressing any of the disclosed nucleic acids. Disclosed are any of the non-naturally occurring disclosed peptides produced by the process of expressing any of the disclosed nucleic acids. Disclosed are any of the disclosed peptides produced by the process of expressing any of the non-naturally disclosed nucleic acids.

Disclosed are animals produced by the process of transfecting a cell within the animal with any of the nucleic acid molecules disclosed herein. Disclosed are animals produced by the process of transfecting a cell within the animal any of the nucleic acid molecules disclosed herein, wherein the animal is a mammal. Also disclosed are animals produced by the process of transfecting a cell within the animal any of the nucleic acid molecules disclosed herein, wherein the mammal is mouse, rat, rabbit, cow, sheep, pig, or primate.

Also disclose are animals produced by the process of adding to the animal any of the cells disclosed herein.

### D. METHODS OF USING THE COMPOSITIONS

1. Methods of Using the Compositions as Research Tools

The disclosed compositions can be used as discussed herein as either reagents in micro arrays or as reagents to probe or analyze existing microarrays. The disclosed compositions can be used in any known method for isolating or identifying single nucleotide polymorphisms. The compositions can also be used in any known method of screening assays, related to chip/micro arrays. The compositions can also be used in any known way of using the computer readable embodiments of the disclosed compositions, for example, to study relatedness or to perform molecular modeling analysis related to the disclosed compositions.

2. Methods of Using the Animal Models

The disclosed animal models are designed such that they have an inflammation related molecule whose expression is controlled by a tissue specific or temporally specific promoter, and the promoter and its activity are typically silent until typically activation of the promoter by removal of a stop sequence, for example, upstream of the promoter. The removal of the stop sequence, for example, occurs typically in the presence of a recombinase such as Cre, because the stop sequence is flanked by recombination sites, such as flox sites. Thus, disclosed are animals that have had germline transmission of one or more of the disclosed nucleic acids. These animals are then placed in the presence of the cognate recombinase in such a way that the recombinase can activate the construct.

The recombinase can be delivered in a variety of ways including somatic gene transfer of a vector encoding the recombinase, such as Cre, crossing with a mouse that contains a germline expressing Cre, which for example is under the control of a tissue specific or temporally specific promoter (it could also be constitutive), or by delivery of Cre itself.

For example, the disclosed Cre producing vectors can be directly injected into, for example, the brain, to activate the neural specific promoter generation of the encoded inflammation related molecule or the Cre producing vectors can be injected in a particular joint, such as the temporal mandibular joint or a knee joint for activation of the bone or collagen specific promoter generation of the encoded inflammation related molecule. (See Figures)

The disclosed models can be used for a variety of purposes including the identification of molecules that modulate the effect of the encoded inflammation related molecule or the inflammation reaction in the model. The disclosed models can also be used to test or verify the effects of a variety of molecules that modulate the effect of the inflammation related molecule or the inflammation reaction in the model.

The models can also be crossed with other models.

It is understood that the disclosed animals include models for arthritis, Alzheimer's, and Parkinson's diseases, as well as inflammatory diseases of the skin.

### E. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

### IL-1β Constructs Related to COLL1A1 Promoters

a) Methods

(1) Construction of an Inducible Interleukin-1β Transgene-IL-1β^{XAT}

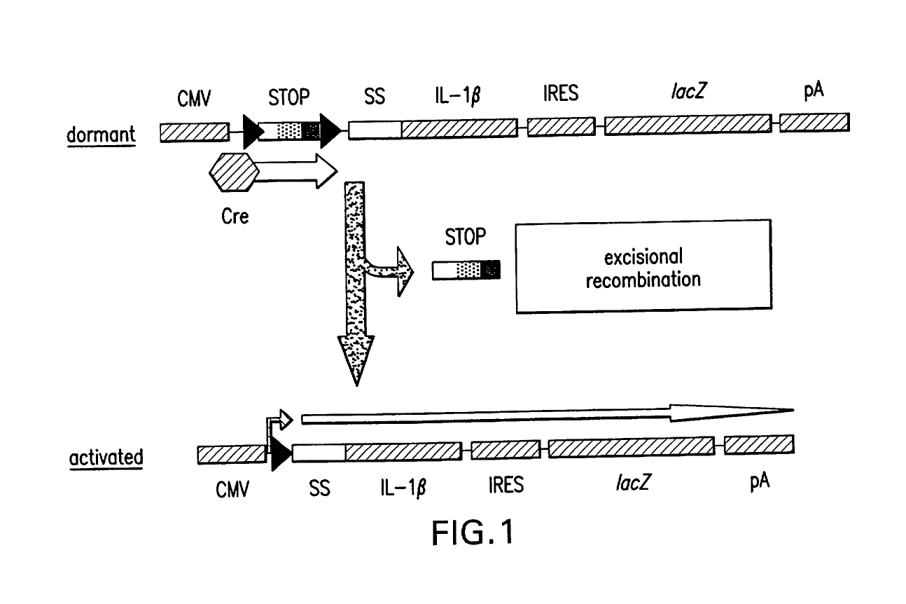

The human IL-1β cDNA was cloned from human U937 cells (ATCC, Manassas Va.) by polymerase chain reaction (PCR) as follows: Total mRNA was extracted employing the TRIzol® reagent (Invitrogen, Carlsbad Calif.) per manufacturer's instructions. PCR primers were designed for the amplification of the portion of the cDNA that corresponds to the mature, secreted IL-1β protein. The peptide secretion signal (ss) of the human IL-1 receptor antagonist gene (IL1-RA) was incorporated into the upper PCR primer, upstream to the IL-1β open reading frame (ORF), to ensure proper compartmentalization and secretion of the transgenic IL-1β peptide [Wingren A G, et al. (1996). Cell Immunol 169:226-37]. This PCR-synthesized ss-IL1β fusion construct was cloned directly into the TOPO 2.1 vector (Invitrogen) per manufacturer's instructions. Subsequently, the cell autonomous and gratuitous β-galactosidase reporter gene (lacZ) was inserted down-stream to the IL-1β ORF, followed by the bovine growth hormone poly A tail (pA) sequence: ssIL-1β-IRES-lacZ bicistronic gene (FIG. 1).

Translation of the second ORF, lacZ, is facilitated by an internal ribosomal entry sequence (IRES) [Havenga M J, et al. (1998) Gene. 222:319-27; Kyrkanides S, et al. (2003). Mol Ther 8: 790-95]. During the initial stages of experimentation, expression of the bicistronic ssIL-1β-IRES-lacZ transgene was ubiquitously driven by the cytomegalovirus promoter (CMV), the transcription of which was inhibited by a loxP-flanked (floxed) transcriptional termination cassette STOP^{fl/fl} [Srinivas S, et al. (2002) BMC Develop Biol 1:4-11]. Transcriptional activation and transgene expression can be turned-on by loxP-directed DNA recombination mediate by the bacteriophage P1 Cre recombinase (Cre/loxP system) [Sternberg N and Hamilton D (1981). J Mol Biol 150: 467-86].

(2) Cre-Mediated Activation of the Inducible IL-1β^{XAT} Transgene

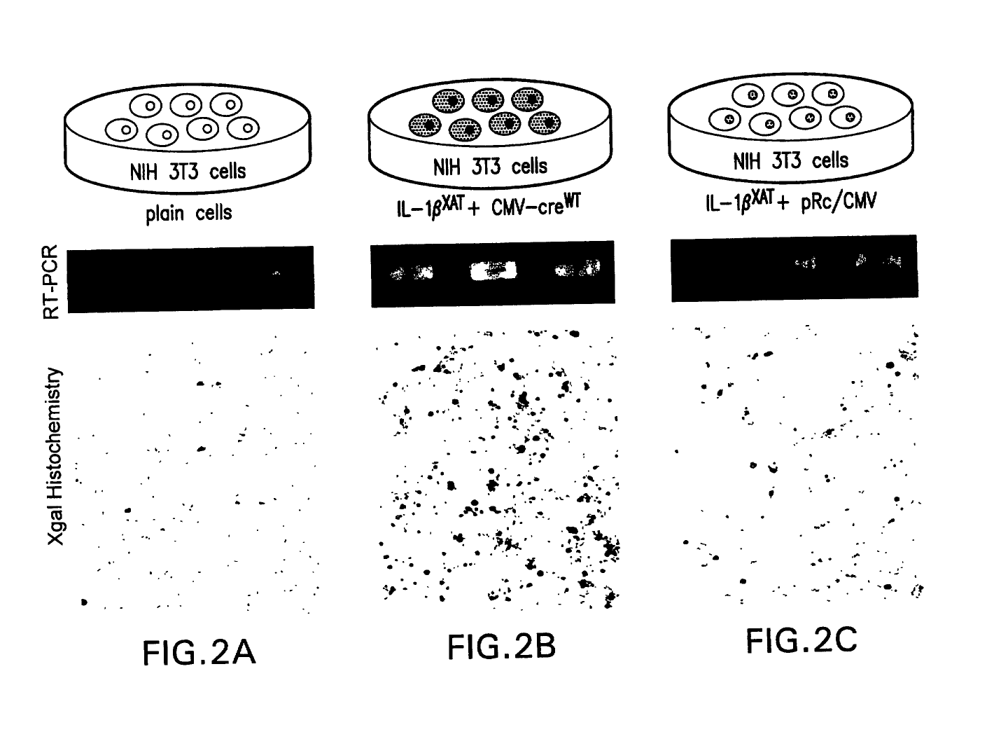

The function of IL-1β^{XAT} was tested in vitro by two different experimental strategies. First, IL-1β^{XAT} regulation by Cre recombinase was evaluated in NIH 3T3 murine fibroblasts (ATCC) in vitro. The IL-1β^{XAT} gene was transiently co-expressed with the wild type cre gene (cloned into the expression vector pRc/CMV-Cre^{WT}; Invitrogen) following transient transfection using the Lipofectamine 2000 reagent (Invitrogen) per manufacturer's instructions. As anticipated, transient expression of Cre recombinase resulted in loxP-directed DNA recombination of IL-1β^{XAT} and excision of the “floxed” transcriptional termination signal [symbol] STOP [symbol] , ultimately leading to gene activation. Control conditions included co-transfection of IL-1β^{XAT} with the expression vector pRc/CMV—(lacking any gene), as well as naïve NIH 3T3 cells. IL-1β expression was assessed at the mRNA level by reverse transcriptase PCR (RT-PCR), and lacZ expression was evaluated by X-gal histochemistry. No IL-1β transcript was detected in naïve NIH 3T3 cells; in contrast, IL-1β^{XAT}+Cre co-transfection resulted in induction of ssIL-1β and lacZ gene expression (FIG. 2).

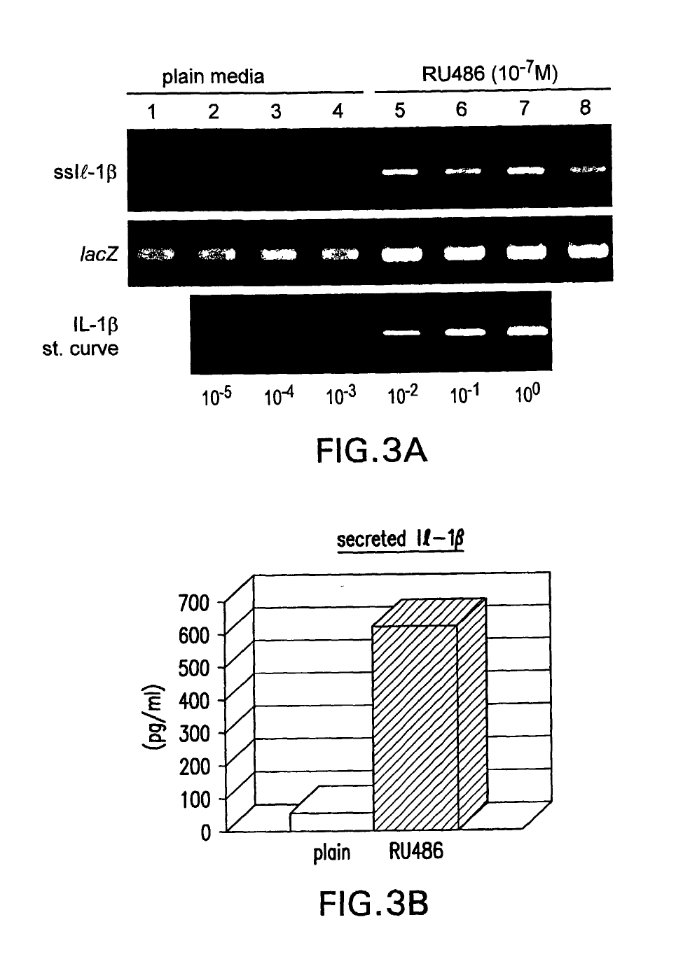

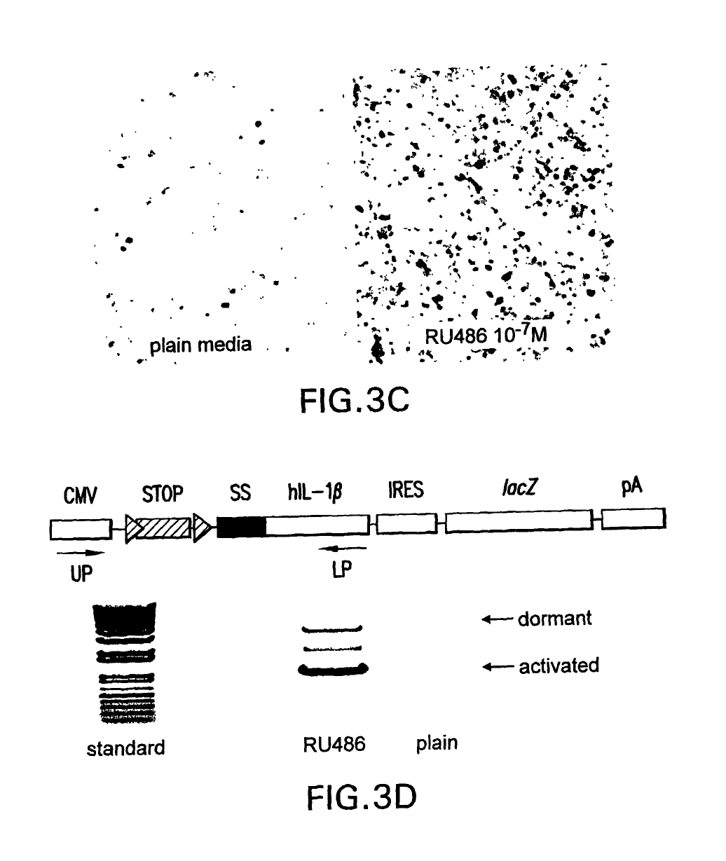

In addition, IL-1β^{XAT} function was evaluated in the inducible Cre recombinase cell line, 293H^{GLVP/CrePr}, a stable cell line recently developed for testing the regulation of excisionally-activated genes utilizing the Cre/loxP technology [Kyrkanides S, et al. (2003). Mol Ther 8: 790-95]. This represents an inducible, loxP-directed DNA recombination system in which Cre recombinase was placed under dual transcriptional and post-translational control. The system is comprised of two components: (1) the chimeric transcriptional activator GLVP and (2) the CrePr fusion protein, which consists of the bacterial Cre recombinase and the mutated progesterone receptor hPR891 gene, driven by a custom GAL4₅/TATA minimal promoter. The mutated hPR891 receptor is highly sensitive to the synthetic progesterone compound mifepristone (RU486): Binding of RU486 to hPR891 results in activation of GLVP and subsequent synthesis of CrePr, the activity of which is also turned-on by RU486 at the post-translational level. RU486 administration to 293HGLVP/CrePr cells following IL-1β^{XAT} transfection resulted in DNA excisional recombination and subsequent expression of human IL-1β and the bacterial β-galactosidase reporter gene (lacZ). Please refer to FIG. 3 for summary of the experiment.

(3) IL-1β^{XAT} Activation Produces a Biologically Potent IL-1β Cytokine

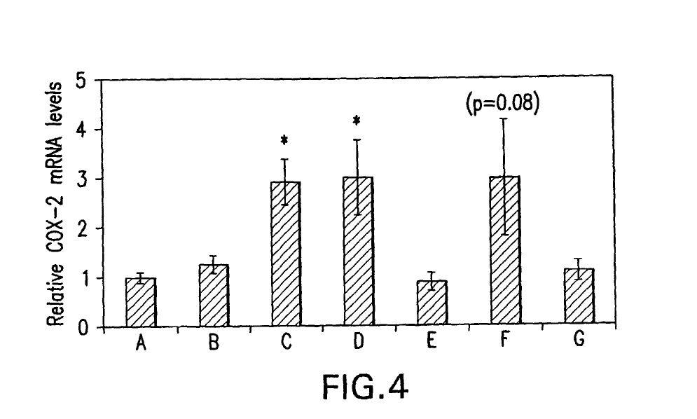

The ability of murine cells to synthesize and secrete biologically active IL-1β cytokine was tested in vitro as follows. In an experiment similar to that described in FIG. 2, above, murine NIH 3T3 fibroblasts were transfected with the IL-1β^{XAT} gene. Concomitantly, Cre recombinase was transiently expressed in these cells (co-transfection of the pRc/CMV-CreWT vector), and the conditioned supernatant media were collected at 72 hours. The presence of human IL-1β was confirmed by ELISA. The conditioned media were then placed on naïve murine fibroblasts, and levels of the inducible cyclooxygenase COX-2 were evaluated as a measure of cytokine potency by quantitative RT-PCR in total mRNA extracts using protocols previously described [Havenga M J, et al. (1998). Gene. 222:319-27; Kyrkanides S, et al. (2003). Mol Titer 8: 790-95; Srinivas S, et al. (2002). BMC Develop Biol 1:4-11; and Sternberg N and Hamilton D (1981). J Mol Biol 150: 467-86]. Control experimental conditions included conditioned media derived from cells co-transfected with the pRc/CMV-backbone vector (lacking the cre gene) along with the IL-1β^{XAT} gene, as well as naïve cells. In brief, murine fibroblasts treated with conditioned medium collected from Cre-activated IL-1β^{XAT} cells resulted in significant COX-2 induction compared to cells exposed to media derived from pRc/CMV-treated or naïve cells. Please see FIG. 4 for a summary of the experiment.

(4) The COLL1A1 Promoter Drives IL-1β^{XAT} Expression to Collagen I Producing Cells

Temporally and spatially controlled expression of IL-1β in mice is accomplished by targeting IL-1β^{XAT} transgene expression to chondrocytes, osteocytes and fibroblasts by the 3.6 Kb promoter of the A1 chain of pro-collagen 1 gene. This promoter has been shown to target gene expression in bone and cartilage [Krebsbach P H, et al. (1993). Mol Cell Biol 13: 5168-74] and was cloned in the IL-1β^{XAT} gene in place of the CMV promoter (FIG. 1):

(COLL1A1-IL1β^{XAT}) COLL1A1 [symbol] STOP [symbol] ssIL1β-IRES-lacZ

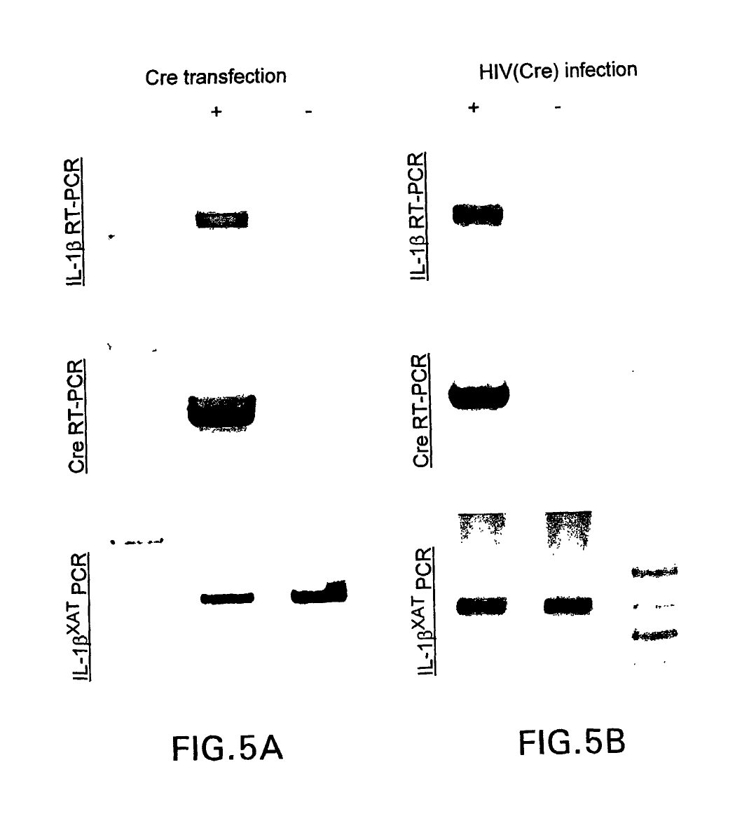

This transgene was constructed and tested in a murine NIH 3T3 stable cell line following expression of Cre recombinase by the transient transfection of the pRc/CMV-Cre^{WT} expression vector or after infection by the lentiviral vector HIV(nlsCre). As anticipated, expression of Cre recombinase led to transgene activation and IL-1β expression. Please refer to FIG. 5 for summary of the experiment.

(5) IL-1β Induces Down-Stream Inflammation-Related Genes

IL-1β is a multi-potent pro-inflammatory cytokine, the expression of which is rapidly upregulated following trauma and/or inflammation. Moreover, a plethora of inflammation-related genes are in turn induced by IL-1β, leading to exacerbation of the inflammatory response. The role of IL-1β in regulating down-stream inflammatory genes, including the inducible isoform of cyclooxygenase (COX-2) and the intercellular adhesion molecule-1 (ICAM-1), the monocyte chemoattractant protein-1 (MCP-1), as well as collagenases A (MMP-2) and B (MMP-9) has been previously examined. ICAM-1 and MCP-1 are molecules associated with the recruitment of circulating immune cells at the site of injury (i.e. neutrophils and monocytes, respectively), whereas MMP-2 and MMP-9 are collagenases associated with tissue destruction during arthritis and injury.

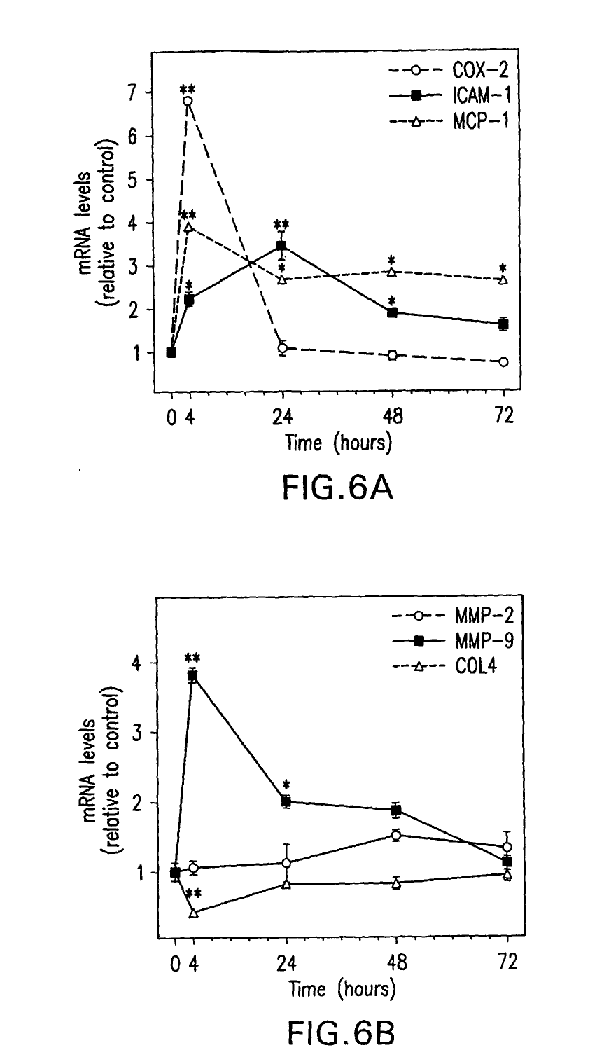

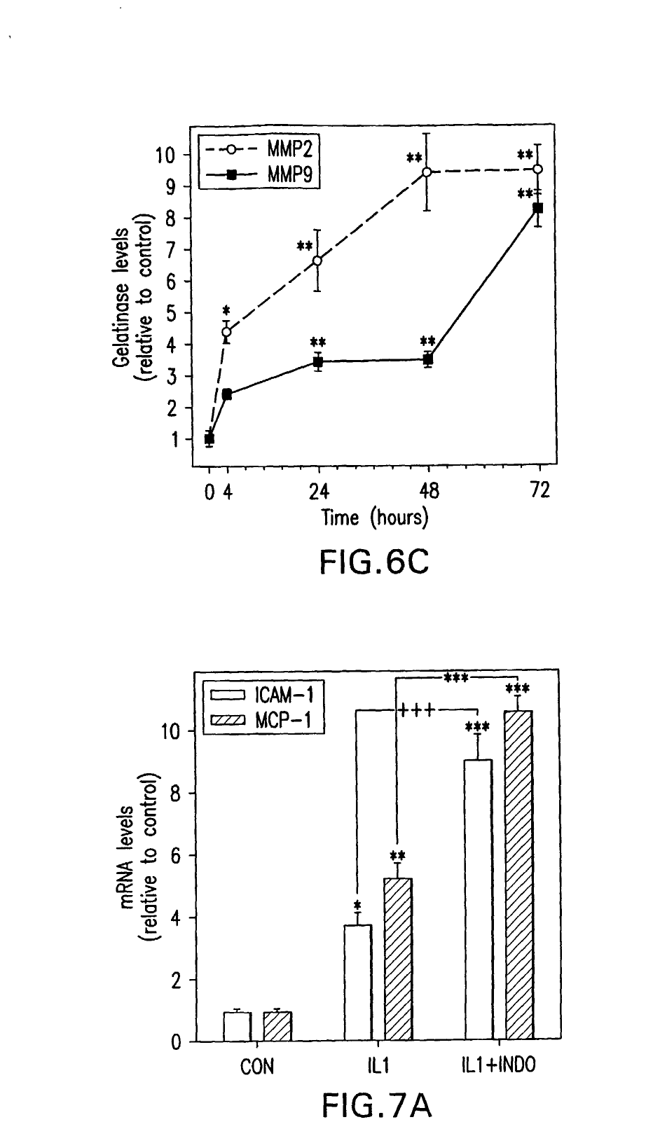

In previous studies [Kyrkanides S, et al. (2003). Mol Ther 8: 790-95], rat endothelial cells were employed as a representative rodent model to investigate the effects of IL-1β on the regulation of ICAM-1, MCP-1, MMP-2 and MMP-9 at the transcriptional level (mRNA) as well as the enzyme activity level (zymography). FIG. 6 summarizes the regulation of these genes over time. IL-1β upregulated the synthesis of COX-2, MCP-1, ICAM-1 and the inducible collagenase B (MMP-9). As anticipated, mRNA levels of the constitutive collagenase B (MMP-2) were not altered by IL-1β, but interestingly MMP-2 enzyme activity also increased with time, presumably due to post-translational activation from other MMPs.

(6) Non-Steroidal Anti-Inflammatory Drugs

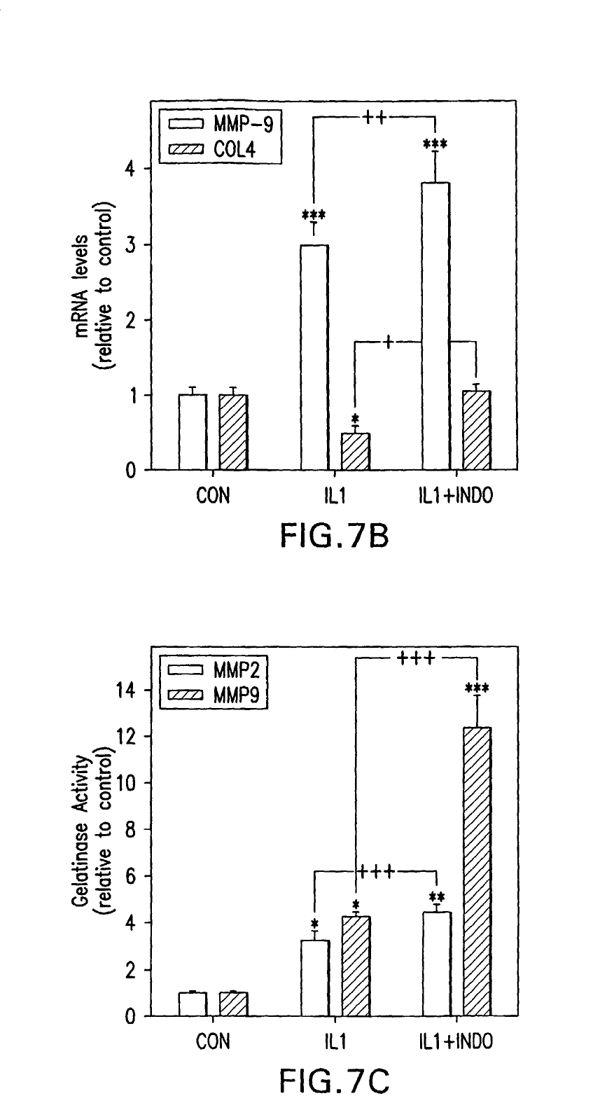

Non-steroidal anti-inflammatory drugs (NSAIDS) are inhibitors of cyclooxygenase, a rate-limiting enzyme in the production of prostaglandins. Indomethacin, a prototype NSAID often employed in laboratory studies as well as in the hospital setting, has been evaluated for the potential to modulate the inflammatory response elicited by pro-inflammatory cytokines, such as IL-1β. To this end, rodent cells were utilized as a model to study the interaction between IL-1β, and indomethacin in vitro [Kyrkanides S, et al. (2000). Am J Orthod Dentofac Orthop 118: 203-09]. Results suggested that the representative NSAID indomethacin further exacerbated the expression of inflammatory mediators induced by IL-1β (FIG. 7), and raised questions about the appropriateness of NSAID administration to patients after tissue injury.

(7) Behavioral Assessment of Orofacial Pain

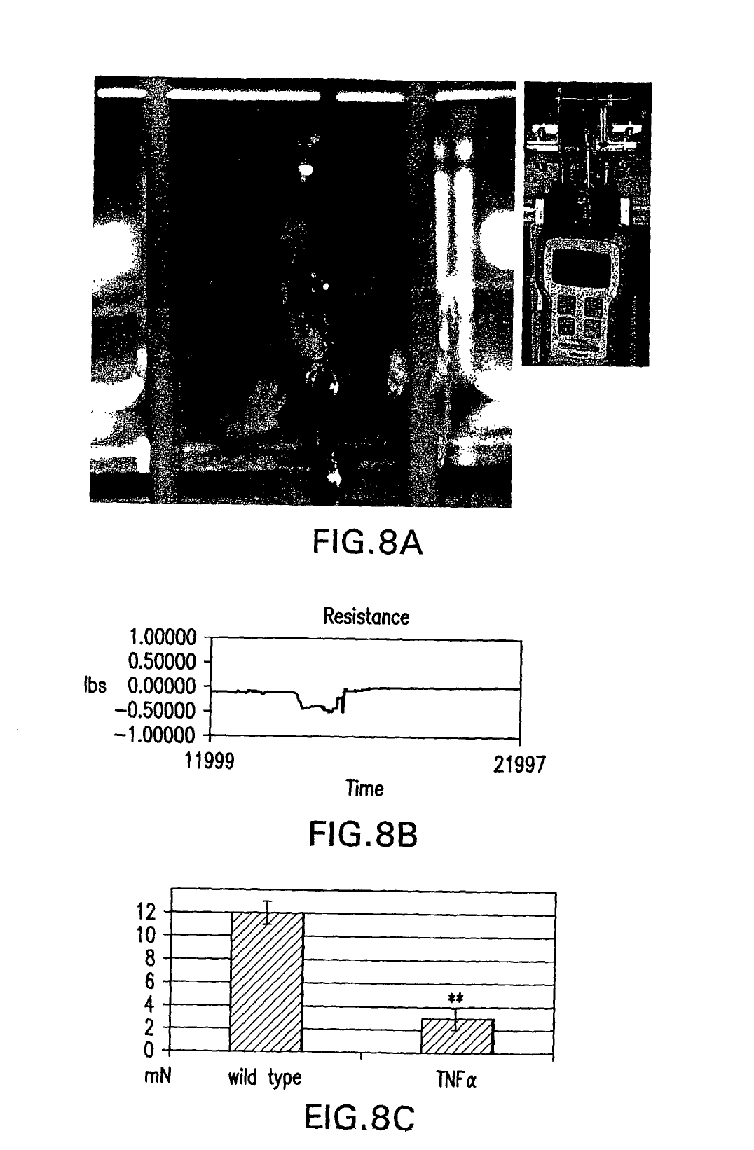

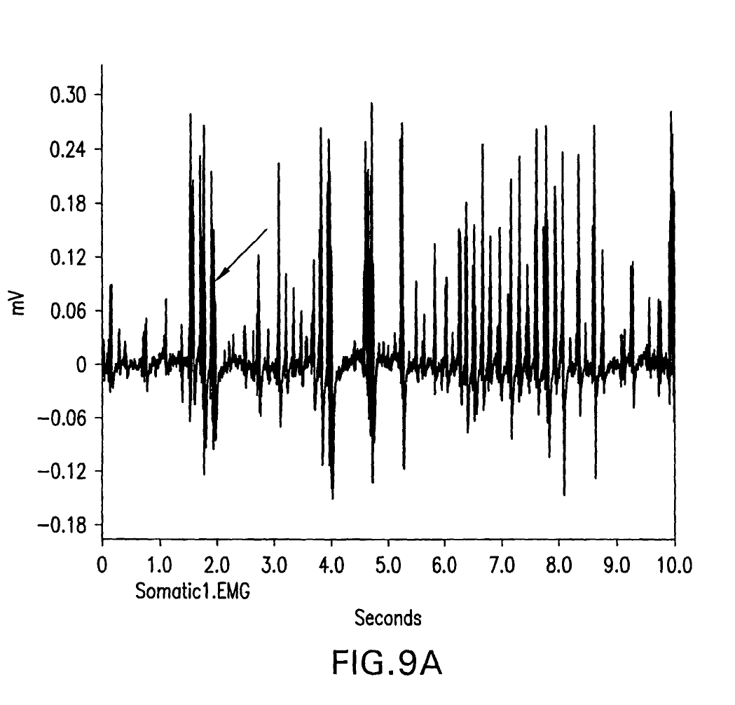

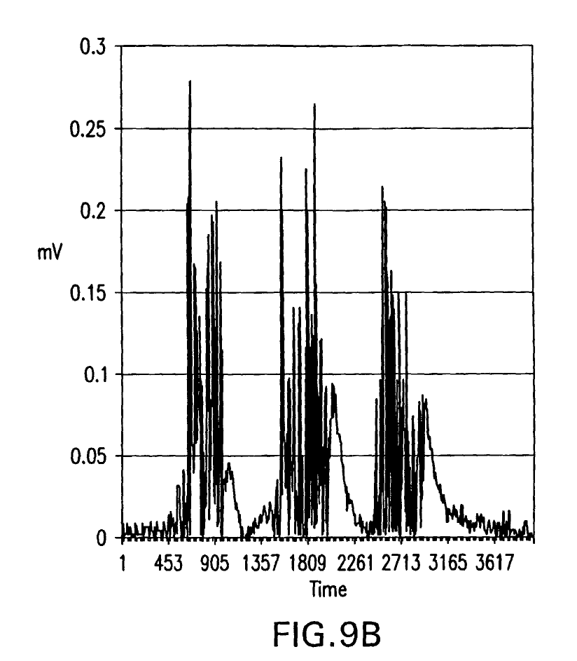

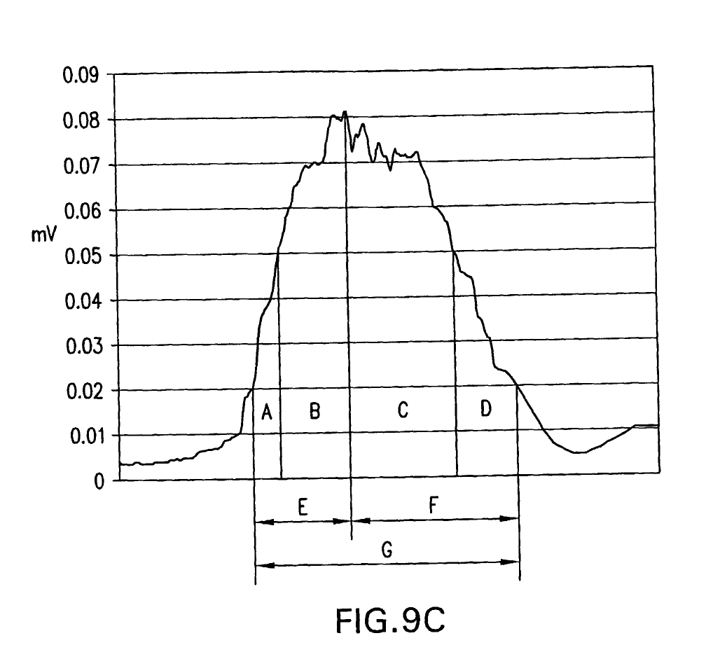

Based on the Pain adaptation model, methods are disclosed for the assessment of pain from the TMJ by measuring changes in resistance to mouth opening as well as electromyographic activity (EMG). Please see FIGS. 8 and 9 for more details.

(8) Development of an IL-1β Somatic Mosaic Model

Sustained IL-1β expression by collagen 1-producing cells, including fibroblasts, chondrocyte and osteocytes, is expected to result in a mouse model of TMJ arthrosis and dysfunction. IL1β^{XAT} regulation is controlled in a temporal (time) and spatial (location) fashion by the Cre/loxP molecular genetic method utilizing (1) a germline transmitted recombinational substrate (COLL1-IL1β^{XAT}) containing a dormant transcription unit and (2) somatic gene transfer of a viral vector that expresses Cre recombinase which “activates” the gene of interest.

Activation of the dormant COLL1-IL1β^{XAT} can be mediated by the transfer of Cre recombinase to the area of interest (TMJ) via a self-inactivating Cre feline immunodeficiency virus FIV(Cre). The effects of this FIV vector system have been previously examined using the reporter gene lacZ (β-galactosidase) in mice that received intra-articular injections of a viral solution [Kyrkanides S, et al. (2004). J Dental Res 83: 65-70], wherein transduction of soft (articular disc) and hard (cartilage) TMJ tissues was demonstrated. The FIV(Cre)vector has been constructed by cloning a loxP-flanked (“floxed”) nlsCre cassette in the place of the lacZ gene; the nuclear localization signal (nls) was fused to the cre open reading frame by PCR and subsequently cloned into the TOPO 2.1 vector (Invitrogen) per manufacturer's instructions employing a custom-made floxed cloning cassette. The reason for developing a self-inactivating cre gene is based on a recent paper [Pfeifer A and Brandon E P, Kootstra Neeltje, Gage F H, Verma I M (2001). Proc Natl Acad Sci U.S.A. 98: 11450-5], whereby the authors reported cytotoxicity due to prolonged expression of Cre recombinase mediated by infection using a lentiviral vector. In the provided construct, upon production of adequate levels of Cre recombinase to produce excisional activation of COLL1-IL1β^{XAT} following successful transduction of target cells with FIV(Cre), Cre is anticipated to de-activate the cre gene by loxP-directed self excisional recombination. This strategy is anticipated to result in activation of COLL1-IL1β^{XAT} by FIV(Cre) avoiding any cytotoxic effects from Cre. Please see FIG. 10.

b) Characterize the Effects of IL-1β Induction in the Temporomandibular Joint

In order to document the role of IL-1β in the development of TMJ arthritis, a transgenic (Tg) mouse harboring IL-1β^{XAT} can be evaluated. Successful gene induction can be determined by an ability to induce sustained secretion of mature IL-1β in the murine TMJ following FIV(Cre) injection into the joint space and subsequent recombinational IL-1β^{XAT} activation. FIV(LacZ), an identical lentiviral vector carrying the reporter gene lacZ instead of cre can serve as a control vector. Expression of IL-1β and other inflammatory mediators associated with arthritis can be spatially characterized in the TMJ. In addition, degenerative changes in the soft and hard tissues of the TMJ can be investigated at the gross and microscopic level.

(1) Characterize the Activation of COLL1-IL1β^{XAT} In Vivo

COLL1-IL1β^{XAT} function can be evaluated after transgene activation in the TMJ of 3 month old mice. Activation of the dormant COLL1-IL1β^{XAT} can be mediated by the transfer of Cre recombinase to the area of interest (TMJ) via the self-inactivating cre feline immunodeficiency FIV(Cre) virus (a total of 10⁵ infectious particles in 50 μl normal saline). The ability of this FIV vector system to transduce TMJ tissues using the reporter gene lacZ (β-galactosidase) has previously examined in mice that received intra-articular injections of a viral solution [Kyrkanides S, et al. (2001). J Neuroimmunol 119: 269-77], wherein the transduction of soft (articular disc) and hard (cartilage) TMJ tissues by the viral vector was demonstrated. The FIV(Cre) transfer vector is described in detail in FIG. 10. It is comprised of a loxP-flanked (“floxed”) nuclear localization signal (nls) fused to the cre gene: nlsCre^{fl/fl}. The reason for developing a self-inactivating nlsCre^{fl/fl} gene is to abolish any cytotoxic effects from the prolonged expression of Cre recombinase mediated in vivo [Pfeifer A and Brandon E P, Kootstra Neeltje, Gage F H, Verma E M (2001). Proc Natl Acad Sci U.S.A. 98: 11450-5]. Upon production of adequate levels of Cre recombinase to produce excisional activation of COLL1-IL1β^{XAT} following successful transduction of target cells with FIV(Cre), Cre protein de-activates the viral nlsCre^{fl/fl} gene by loxP-directed self excisional recombination. COLL1-IL1β^{XAT} function can be evaluated as follows. (1) First, lacZ expression can be readily assessed in decalcified TMJ histology sections by X-gal histochemistry and immunocytochemistry. (2) ssIL-1β and lacZ transcript levels is assessed by semi-quantitative RT-PCR in TMJ total mRNA extracts from experimental and control animals. Localization of ssIL-1β and lacZ mRNA can be achieved on TMJ histology sections by in situ hybridization (ISH); the identity of transduced cells can be confirmed by coupling ISH with immunocytochemistry (ICC). Osteocytes/osteblasts can be confirmed by the expression alkaline phosphatase, osteocalcin, or type I collagen. Chondrocytes can be confirmed by detection of collagen II. (3) Human IL-1β protein expression can be analyzed in TMJ homogenates by ELISA (Catalog #DLB50; R&D Systems Inc, Minneapolis Minn.).

(2) Investigate the Inflammatory Effects of IL-1β Induction in the TMJ

The effects of IL-1β expression in the TMJ can be studied in 3 month old COLL1-IL1β^{XAT} transgenic mice at 4 weeks following intra-articular injection of FIV(Cre) virus (a total of 10⁵ infectious particles in 50 μl normal saline). The FIV(lacZ) vector, capable of transducing soft and hard TMJ tissues with the reporter gene lacZ, can be administered to transgenic mice; an additional group of mice can receive FIV(lacZ)-injections and serve as controls [Kyrkanides S, et al. (2004). J Dental Res 83: 65-70]. Please see Table 3 for summary of experiment. Based on the properties of the somatic mosaic model, it is anticipate that intra-articular transfer of Cre recombinase into the TMJ of COLL1-IL1β^{XAT} transgenic mice can result in sustained expression of human IL-1β by infected chondrocytes, osteocytes and fibroblasts. In contrast, FIV(lacZ)-injected mice lack human IL1β expression. Saline-treated mice (50 μl) can be included and control for the effects of the virus injection.

Since IL-1β is a multipotent cytokine known to induce a number of down-stream inflammation-related genes, the expression of cytokines (TNFα, IL-6, murine IL-1β), adhesion molecules (ICAM-1, VCAM-1), chemokines (MCP-1), collagenases (MMP-3, MMP-9) can be examined at the mRNA and protein level as previously described [Havenga M J, et al. (1998) Gene. 222:319-27; Kyrkanides S, et al. (2003) Mol Ther 8: 790-95; Srinivas S, et al. (2002) BMC Develop Biol 1:4-11; and Sternberg N and Hamilton D (1981). J Mol Biol 150: 467-8] in FIV(Cre), FIV(lacZ) and saline treated mice. In addition, the levels of the inducible COX-2 can be measured at the mRNA and protein levels as previously described [Maguire-Zeiss K A, et al. (2002). Neurobiol Aging 23:977-84 and Sternberg N and Hamilton D (1981). J Mol Biol 150: 467-86], as well as production of prostaglandin PGE₂ [O'Banion M K, et al. (1991). J Biol Chem 266: 23261-7; O'Banion M K, et al. (1992). Proc Natl Acad Sci U.S.A. 89:4888-92; and O'Banion M K (1999). Crit. Rev Neurobiol 13: 45-82]. Moreover, TMJ morphology can be assessed in H&E-stained histology sections as follows. Degenerative changes in the articular cartilage can be evaluated and graded in sagittal sections examined under light microscope, and scored into five categories according to Wilhelmi and Faust [Wilhelmi G and Faust R (1976) Pharmacol 14:289-96] and Helminen et al. [Helminen H J, Kiraly et al. (1993) J Clin Invest 92:582-95]: grade 0, no apparent changes; grade 1, superficial fibrillation of articular cartilage; grade 2, defects limited to uncalcified cartilage; grade 3, defects extending into calcified cartilage; and grade 4, exposure of subchondral bone at the articular surface. Each TMJ can be graded according to the highest score observed within the serial sections.

TABLE 3
Transgene expression in COLL1-IL1β^{XAT} transgenic mouse lines.

| Treatment | Animals (N) | Analysis | Methods |
|---|---|---|---|
| 1. FIV(Cre) | 8 × 5 lines = 40 | mRNA (3 mice) | QRT-PCR |
| 2. FIV(lacZ) | 8 × 5 lines = 40 | Protein (3 mice) | ELISA |
| 3. Saline | 8 × 5 lines = 40 | Histology | Immuno/Histo- |
| | | (2 mice) | chemistry, in |
| | | | situ hybridization |
| | TOTAL mice = 120 | | |

3-5 mouse lines can be analyzed for transgene function at the mRNA, protein and histology levels. The founders can be mated with C57BL/6/wild types and their offspring (N = 8) can be injected intra-articularly with FIV(Cre), FIV(lacZ) or saline at 3 months of age and subsequently analyzed 4 weeks later.

(3) Statistical Analysis

IL-1β expression between the three treatment groups, FIV(Cre), FIV(lacZ) and saline, can be compared for each mouse line. This can be done using a nonparametric ANOVA (Kruskal-Wallis test). Similarly, one can assay whether treatment affects expression of other genes, including murine TNFα/IL-6/IL-1β, MMP's and COX-2. TMJ morphology for each mouse can be summarized with a score from 0 to 5. Mean morphology scores across treatment groups can be compared using nonparanietric ANOVA. In each case significance levels can, for example be set at 0.05.

The expression of IL-1β can be correlated with expression of other inflammation-related molecules, as well as with morphology. As descriptive measures, scatter plots and calculate Spearman's rank correlation can be produced between IL-1β expression and the expression of each of the other genes. This can be done separately for each mouse line. The data can then be pooled together to formally test the hypotheses. Specifically, statistical significance can be determined based on a linear mixed model, where each mouse line is a cluster, the outcome is the expression of a gene such as COX-2, and the covariate is the expression of IL-1β. Linear mixed models are an extension of linear regression models to allow correlation (here due to some mice sharing a common line). A similar approach can be taken for measuring associations between gene expression and morphology.

c) Determine the Role of IL-1β in the development of Temporomandibular Disorders [TMJD MOUSE]

Based on clinical findings in human patients, the development of hyperalgesia and nociception associated with jaw function in the IL-1β^{XAT} transgenic mice can occur, which can be assessed behaviorally by measuring changes in resistance to mouth opening, electromyographic activity of masticatory muscles and other behavioral pain indicators. Changes in expression of neurotransmitters implicated in pain transmission can be evaluated in peripheral (TMJ) and central (trigeminal ganglia & brain stem sensory nuclei) tissues at the protein and mRNA levels. The data generated in these experiments can be correlated with the levels of IL-1β in the TMJ and course of time.

It is believed that chronic expression of IL-1β in the TMJ can lead to the development of temporomandibular disorders (TMJD) in the mouse. Patients presenting with TMJD can have one or more of an array of clinical features, including increased pain from the TMJ during orofacial function, limitation of jaw opening, as well as decreased maximal clench and chewing amplitude of electromyographic activity of the masticatory (masseter and temporal) muscles. Additional behavioral features include rubbing of the area of pain as well as flinching of the head. Lund et al. [Lund J P, et al. (1991). Can J Physiol Pharmcol 69:683-694] have formulated the Pain Adaptation Model to explain the clinical features seen in musculoskeletal pain conditions. The principal features of the Pain Adaptation Model suggests that in the presence of nociceptive input to the motor program and brainstem interneurons, there is a decrease in muscle strength in concentric muscle work (chewing, clenching), a reduced range of motion and a slowing of movement due to antagonistic co-contraction of extensors during eccentric muscle work.

It is believed that (1) pain has general effects that include changes in posture and facial expressions, (2) motor effects are independent of the type of tissue in which pain arises, (3) reduced agonist muscle output is encountered in concentric (shortening contraction) muscle work, and (4) co-contraction of muscle antagonists occurs during muscle extension. The somatic mosaic analysis method in the COLL1-IL1β^{XAT} transgenic mouse can be used to determine whether sustained expression of IL-1β in the TMJ results in the development of TMJD in the mouse.

(1) Clinical Evaluation of Orofacial Pain from the TMJ in the Mouse

The following methods can be employed as measures of orofacial pain: (1) flinching and rubbing of the face, (2) electromyographic (EMG) activity of the masticatory muscles, and (3) resistance to mouth opening. These methods are believed to replicate behavioral and somatic events seen in human patients with TMJD pain. Utilizing the aforementioned 3 methods, pain can be evaluated over 24 weeks in COLL1-IL1β^{XAT} transgenic mouse lines following FIV(Cre), FIV(lacZ) or saline intra-articular injection at 8 weeks of age, for example.

Behavioral testing sessions can take place between 08:00 and 17:00 h in a quiet vivarium room maintained at 23° C. First, head flinching and face rubbing can be evaluated. To this end, each animal can be placed in a custom-made observation chamber (12×12×12 inch) with mirrored-glass walls on 3 sides; a digital video camera can record each session and provide documentation. Bedding from the animals' cage can be carried into the observation chamber to minimize environment-induced stress. The animals can be allowed a 30 min habituation (adaptation) period in the observation chamber to minimize stress [Abbott F V, et al. (1986) Eur J Pharmacol 126: 126-41]. The mice typically may not have access to food or water during the test. Fifteen consecutive 3-minute sessions can be be recorded and evaluated: Rubbing of the face (scored as seconds the animal exhibits the behavior in a 45 min. session) and head flinching (scored as the number of times the animal exhibits the behavior in a 45 min. session) can be assessed as previously described [Calvelou P, et al. (1995). Pain 62:295-301]. Behavioral analysis can be made by a blinded investigator as to the mouse group assignment.

Second, electromyographic (EMG) signals can be obtained with a telemetry system using a fully implantable device that combines continuous registration of one biopotential (right masseter muscle). This wireless transducer can be implanted in Tg mice (N=10) at 6 weeks of age. These mice can be sacrificed at the 24 week time point, so that EMG longitudinal data can be recorded on each mouse at 4-8-16-24 weeks after Tg induction.

Third, resistance to mouth opening can be evaluated in terminal animals as follows. The mice can be anesthetized with CO₂ (60%)/O₂ (40%) mixture under constant pressure of 25 psi, a method that provides approximately 5 min anesthesia: CO₂ is quickly cleared from the animal via exhalation with minimal physiological changes suitable to the methods. During the anesthesia period, the animals can be mounted on a custom restraining device and prepared for a series of resistance to jaw opening recordings. For this purpose, the head is stabilized by the restraining device, whereas the mandible can be extended vertically by depressing the force gage at 5 mm increments. Previous experiments have demonstrated that the animal will attempt to close the mouth when the mandible is depressed. In brief, an orthodontic Kobayashi hook can be temporarily bonded to the mandibular incisors and further be attached to the digital dynamometer (FGF series, Kernco Instruments) wired to a DELL PC computer through an A/D conversion card (NIO16E1, National Instruments). A series of 5 recordings an be collected by the LabView software package (National Instruments, Austin Tex.) at 5, 10, 15, 20 and 25 mm of mandibular vertical opening. These data can be analyzed after the experiment is completed.

(2) Central Nervous System Changes

The mandibular division of the trigeminal nerve provides sensory innervation to the TMJ. The cell bodies of these primary sensory neurons are located in the posterolateral portion of the trigeminal ganglion extending unmyelinated (C-fibers) or thinly myelinated (Aδ-fibers) peripheral projections to structures of the face and jaws. Inflammation, injury or other agents may cause excitation of their free and un-specialized nerve endings, which are predominately involved in the transmission of nociception from the TMJ [Sessle B J and Hu J W (1991) Can J Physiol Pharmacol 69: 617 626]. The central projections enter the brain stem via the ventrolateral pons, descend caudally as the trigeminal tract and synapse with second order sensory neurons at the substantia gelatinosa of the subnucleus caudalis of the descending trigeminal nucleus (medullary dorsal horn). Second order sensory neurons extend projections to the nucleus proprius, followed by subsequent projections to the intermedial gray, and then to the reticular formation of the brain stem, and through the intralaminar nuclei of the thalamus project wide spread connections into the cortex. A number of small neuropeptides, such as substance P(SP) and calcitonin-gene related peptide (CGRP), have been implicated in the transmission of pain from the periphery to the central nervous system (CNS) [Kyrkanides S, et al. (2002). J Orofac Pain 16:229-35]. It is expected that sustained expression of IL-1β in the mouse TMJ elicits, in addition to a peripheral inflammatory response, changes in the expression of neurotransmitters in the CNS, including the trigeminal ganglion as well as the descending trigeminal nucleus.

Following the assessment of resistance to mouth opening (described above), the mice can be deeply anesthetized by pentobarbital (100 mg/kg) intraperitoneal administration and removed from the restraining device. A subgroup of mice can be decapitated and their trigeminal ganglia, brain stem and TMJ can be harvested and snap frozen. Another subgroup of mice can be terminated via transcardial perfusion of 50 ml of 4% paraformaldehyde solution in PBS pH=8.0. The trigeminal ganglia, brain stems and TMJ can be harvested and frozen until processed. The expression of SP and CGRP can be studied at the mRNA and protein levels. In brief, mRNA levels can be evaluated by quantitative RT-PCR in total RNA extracts from tissue homogenates using the TRIzol reagent (Invitrogen) per manufacturer's instructions. For this purpose, methods can be adopted as previously described [Kyrkanides S, et al. (1999) J Neuroimmunol 95:95-106; Kyrkanides S, et al. (2000) Am J Orthod Dentofac Orthop 118: 203-09; Kyrkanides S, et al. (2001) J Neuroimmunol 119: 269-77; and Kyrkanides S, Moore et al. (2002) Mol Brain Res 104: 159-69]. Protein levels of expression can be assessed semi-quantitatively by immunocytochemistry as previously described [Kyrkanides S, et al. (2002) J Orofac Pain 16:229-35]. 1In brief, fixed brain tissues can be cut on a freezing microtome in 18 μm thick sections that can be collected onto coated-glass slides. Tissue sections can be processed by immunocytochemistry employing antibodies raised against SP and CGRP. Control sections for antibody specificity can be processed simultaneously in the absence of primary antibody. All tissue can be processed simultaneously and all images captured taken using identical illumination and exposure. Histologic microphotographs can be captured by a SPOT CCD camera attached on a BX51 Olympus microscope and connected to a DELL PC computer. One investigator can be blinded as to group of animals studied and can perform the analysis using the NIH Image software program. The data can be recorded as number of immunoreactive pixels per microscopic field. The change of immunoreactivity in brain sections can be expressed as the relative change in immunoreactivity recorded in the right versus the left (no treatment) side in every section studied (left-right/left). Anatomical designations of the different regions examined in reference to the trigeminal nuclear complex [Kyrkanides S, et al. (2002) J Orofac Pain 16:229-35]. Averages can be calculated at each level of the brain stem for the animals in experimental and control groups.

(3) Peripheral Inflammation—TMJ pathology

Since IL-1β is a multipotent cytokine known to induce a number of down-stream inflammation-related genes, the expression of murine cytokines (TNFα, IL-6, IL-1β), adhesion molecules (ICAM-1, VCAM-1), chemokines (MCP-1), collagenases (MMP-3, MMP-9) can be evaluated at the mRNA and protein level, by quantitative RT-PCR and immunocytochemistry as previously described [Kyrkanides S, et al. (1999) J Neuroimmunol 95:95-106; Kyrkanides S, et al. (2000) Am J Orthod Dentofac Orthop 118: 203-09; Kyrkanides S, et al. (2001) J Neuroimmunol 119: 269-77; and Kyrkanides S, Moore et al. (2002) Mol Brain Res 104: 159-69]. In addition, levels of the inducible COX-2 at the mRNA and protein levels can be measured as previously described, as well as production of prostaglandin PGE₂ [O'Banion M K, et al. (1991) J Biol Chem 266: 23261-7 and O'Banion M K, et al. (1992) Proc Natl Acad Sci U.S.A. 89:4888-92]. Moreover, TMJ morphology can be assessed in H&E-stained histology sections as follows. Degenerative changes in the articular cartilage can be evaluated and graded in sagittal sections examined under light microscope, and scored into five categories according to Wilhelmi and Faust [Wilhelmi G and Faust R (1976) Pharmacol 14:289-96] and Helminen et al. [Helminen H J, et al. (1993) J Clin Invest 92:582-95]: grade 0, no apparent changes; grade 1, superficial fibrillation of articular cartilage; grade 2, defects limited to uncalcified cartilage; grade 3, defects extending into calcified cartilage; and grade 4, exposure of subchondral bone at the articular surface. Each TMJ can be graded according to the highest score observed within the serial sections.

The presence of inflammatory cells, including neutrophils, monocytes/macrophages and lymphocytes in the joint can be investigated at the histology level by immunocytochemistry and double immuno-fluorescence as previously described [Kyrkanides S, et al. (1999) J Neuroimmunol 95:95-106; Kyrkanides S, et al. (2000) Am J Orthod Dentofac Orthop 118: 203-09; Kyrkanides S, et al. (2001) J Neuroimmunol 119: 269-77; and Kyrkanides S, Moore et al. (2002) Mol Brain Res 104: 159-69] in experimental and control mice sacrificed 4-8-16-24 weeks after treatment. In brief, neutrophils can be detected by a rat anti-murine neutrophil antibody (MCA771 GA; Serotec, Raleigh, N.C.); monocytes & macrophages can be stained with a rat anti-mouse CD11b antibody (MC A74; Serotec Inc); activated cells can be immunolocalized by a rat anti-major histocompatibility complex class-II antibody (MHC-II; Bachem, Torrance, Calif.; clone ER-TR3). Lymphocytes can be detected by a monoclonal antibody raised against CD3 (MCA 1477; Serotec). Quantification of the number of cells can be described both in terms of number of positive cells per field [Kyrkanides S, et al. (2003) Mol Brain Res 119: 1-9], as well as staining profile [Kyrkanides S, et al. (2002) J Orofac Pain 16:229-35].

The levels of IL-1β expression can also be temporally characterize at the mRNA level by RT-PCR in TMJ total RNA extracts, as well as at the protein level by ELISA in TMJ homogenate extracts harvested from experimental and control mice. Histologically, ssIL-1β mRNA localization can be performed by in situ hybridization (ISH); the identity of transduced cells can be confirmed by coupling ISH with immunocytochemistry (ICC), employing antibodies raised against the following antigens: Osteocgtes/osteblasts can be confirmed by the expression alkaline phosphatase, osteocalcin and type I collagen [Liu F, et al. (1997) Exp Cell Res 232: 97-105 and Adamo C T, et al (2001) J Oral Implantol 27: 25-31]. Chondrocytes can be confirmed by the expression of collagen II [Scott-Burden T, et al. (2002) Ann Thorac Surg 73: 1528-33]. Localization of gene expression can also be assessed by the reporter gene lacZ by means of Xgal histochemistry.

Since the introduction of FIV proteins can elicit an immunologic response in mice treated with FIV vectors, the host's immunologic response can be characterized following FIV intra-articular injection. The presence (titers) of antibodies against viral and transgenic proteins can be quantitatively assessed in blood serum at the different experimental time points. To this end, IgG and IgM titers for the FIV p24 antigen as well as human IL-1β can be assessed by customized ELISA method. In brief, ELISA plates can be coated with 5 μg of human IL-1β (Sigma; St. Louis Mo.) or p24 recombinant proteins (IDEXX Laboratories Inc.; Westbrook Me.). After incubation with the test sera, the plates can be incubated with alkaline phosphatase-conjugated goat anti-mouse IgG and IgM (Southern Biotechnology Associates, Inc; Birmingham Ala.). Antibody titers can be established as the serum dilution that reached absorbance levels (at 405 nm) of saline injected mice assuming linear extrapolation [Kang Y, et al. (2002). J Virol 76 9378-88].

(4) Experimental Conditions

The effects of IL-1β expression in the TMJ can be studied in COLL1-IL1β^{XAT} transgenic mice over time (4-8-16-24 weeks) after intra-articular injection of FIV(Cre), FIV(lacZ) or saline. Injection can be unilateral or bilateral. The advantage of a unilateral injection is that is allows for the contralateral side to be employed as an internal control for the various procedures and measures [Kyrkanides S, et al. (2002). J Orofac Pain 16:229-35]. Please see Table 4, below, for a summary. In brief, it is anticipated that, based on the properties of the somatic mosaic model, intra-articular transfer of Cre recombinase to the TMJ of COLL1IL1β^{XAT} transgenic mice can result in sustained expression of human IL-1β by infected cells. In contrast, it is expected that FIV(lacZ)-injected mice would lack detectable human IL1β expression. Saline-treated mice can also be included to serve as controls. After intra-articular injections, the animals can be returned to their cages and studied clinically as described above. In brief, all animals can be evaluated for behavioral changes associated with head flinching and rubbing of the face at each time point (4-8-16-24 weeks). In addition, at each time point, a subgroup of animals can be terminated; these animals can be evaluated for resistance to mouth opening as described above. Lastly, one group of mice can be maintained for 24 weeks. These animals can be subjected to the incorporation of the wireless EMG transducer and can be utilized for obtaining EMG measurements at each of the time points, until sacrificed at 24 weeks after FIV treatment. For each subgroup of killed mice, the various tissues of interest can be harvested for further analysis as described above. In total, 3 groups of transgenic mice (cre, lacZ, saline) originating from the two founder lines can be utilized. Each group consists of 40 mice. Ten mice of each group can be sacrificed at each of the 4 time points (4-8-16-24 weeks): 5 for harvesting fresh and 5 for fixed tissues, a total of 240 Tg mice.

TABLE 4
Characterize the effects of IL-1β in the TMJ

| Mouse Lines | Groups | Time Points | Animals (N = 10) | Methods |
|---|---|---|---|---|
| “high” IL-1β | FIV(Cre) | 4-8-16-24 | 10 × 4 time points = 40 | QRT-PCR |
| “high” IL-1β | FIV(lacZ) | 4-8-16-24 | 10 × 4 time points = 40 | ELISA |
| “high” IL-1β | saline | 4-8-16-24 | 10 × 4 time points = 40 | Histology |
| “moderate” IL-1β | FIV(Cre) | 4-8-16-24 | 10 × 4 time points = 40 | Behaviora |
| “moderate” IL-1β | FIV(lacZ) | 4-8-16-24 | 10 × 4 time points = 40 | |
| “moderate” IL-1β | saline | 4-8-16-24 | 10 × 4 time points = 40 | |
| | | | Total mice = 240 | |

The effects of FIV(Cre), FIV(lacZ) and saline intra-articular injection can be analyzed in two COLL1-IL1β^{XAT} transgenic mouse lines: one characterized by relatively “high” levels of IL-1β expression following FIV(Cre) intra-articular injection and a second mouse line with “moderate” IL-1 β expression. The effects of IL-1β expression in the TMJ in these two mouse lines can be evaluated over time (4-8-16-24 weeks) following treatment: FIV(Cre), FIV(lacZ) and saline injection. To this end, ten mice can be sacrificed at each time point and be studied by molecular, histological and behavioral methods.

(5) Statistical Analysis

Within each treatment group and line, nonparametric ANOVA methods can be used to test whether behavioral measures (rubbing of face, head flinching and resistance to mouth opening), central nervous system changes (SP and CGRP) and peripheral inflammation differ across the four time points. Similarly, comparisons can be made between treatment groups at each time. Mice in the 24 week group can have EMG measurements taken at each of the 4 time points plus baseline. EMG can be compared both across time and between groups using a linear mixed model (separately for each line), where each mouse is a cluster, EMG is the response, and time and treatment group indicators are covariates.

If, as anticipated, there are more behavioral changes in the FIV(Cre) group, one of the two mouse lines can be selected. Looking only at the FIV(Cre) group data, the line that tended to have less bite force, higher EMG activity, increased face rubbing and head flinching activity can be selected.

(6) Anticipated Results

Based on clinical findings from human patients and clinical research the development of hyperalgesia and nociception associated with mouth opening secondary to chronic expression of IL-1β in the TMJ is expected. These behavioral changes can be documented through the proposed behavioral assessment methods described herein. In addition, an increase in the synthesis of neuropeptides in the trigeminal ganglion as well as changes in their levels of expression at the descending trigeminal nucleus of the brain stem as well as the TMJ is anticipated. Changes in SP levels of expression in the rabbit brain stem following experimental TMJ nociception have been demonstrated [Kyrkanides S, et al. (2002). J Orofac Pain 16:229-35].

Bilateral transgene induction by injecting FIV(Cre) in both TMJs can also be performed. Capsaicin, an algesic chemical widely utilized in pain research, to induce nociception in the COLL1-IL1β mice. In brief, capsaicin can be administered in the TMJ in conjunction with IL-1β^{XAT} activation to produce experimental nociception. In this latter scenario, it is anticipated that chronic expression of IL-1β in the TMJ can confer a decrease in the pain threshold of mice elicited by low doses of capsaicin [Kyrkanides S, et al. (2002). J Orofac Pain 16:229-35].

Intra-articular administration of FIV may result in the development of an inflammatory response into the TMJ due to the proteins of the virus itself, or the bacterial Cre recombinase. In such case, it may be observed that infiltration of immune cells into the TMJ that normally are not found there. Based on previous results employing FIV(lacZ) in mice [Kyrkanides S, et al. (2004). J Dental Res 83: 65-70] and because of the small amount of virus injected, no inflammatory response is expected (data not shown). Nevertheless, one can control for the effects of FIV by including animals receiving FIV(lacZ) injections and comparing them to the experimental FIV(Cre) mice. Moreover, Cre recombinase can be delivered by a self-inactivating vector, whereby the cre gene can be excised and removed, therefore minimizing any chance for inflammatory response to Cre recombinase. In addition to the presence of inflammatory cells in the TMJ, one can also investigate the presence of antibodies produced in response to the FIV injection.

### Example 2

### COX-2 Related Constructs and Mice

### The Role of COX-2 in the Development of IL-1β Induced Arthritis and TMJD

IL-1β is an inducer of cyclooxygenase-2 (COX-2), a key rate-limiting enzyme in the production of prostanoids during inflammation. COX-2 is of particular therapeutic interest since it is the target of commercially available over-the-counter and prescription drugs often utilized in cases of arthralgia for the management of pain. To this end, TMJ pathology and behavior can be investigated in IL-1β^{XAT} mice treated with a COX-2 selective inhibitor. A COX-1 (constitutive isoform) selective inhibitor and a mixed inhibitor (COX-1 & COX-2) can also be employed as controls. The outcome data can be analyzed relative to IL-1β levels and the time course of the disorder. To confirm the role of the COX in TMJD, the IL-1β^{XAT} Tg mice can be crossed with COX-2, as well as COX-1, knockout mice, and the effects of conditional induction of IL-1β in the TMJ investigated as previously described. This can provide valuable information on the effectiveness of pharmacologic inhibitors in attenuating or possibly exacerbating the development of temporomandibular joint disorders.

Anti-inflammatory drugs, primarily over the counter non-steroidal (NSAIDs) such as ibuprofen (i.e. Advil®), naproxen (i.e. Alleve®), salicylates (i.e. aspirin), and others are commonly utilized by patients for the management of symptoms arising from inflammation of the TMJ and other joints. In addition, the discovery and implication of the inducible prostaglandin H₂ synthase, COX-2, in inflammation [O'Banion M K, et al. (1991) J Biol Chem 266: 23261-7 and O'Banion M K, et al. (1992) Proc Natl Acad Sci U.S.A. 89:4888-92; O'Banion M K (1999) Crit. Rev Neurobiol 13: 45-82] led to development of COX-2 selective inhibitors (i.e. Celebrex®, Vioxx®), drugs that offer new alternatives for the management of chronic arthritic pain. Interestingly, despite the fact that the use of NSAIDs dominates the clinical arena of joint inflammation, limited attention has been given to the drugs' long-term effects on disease morbidity. For example, as seen in FIG. 7, NSAIDs may in fact exacerbate an inflammatory condition when administered inappropriately. It has also been reported that COX-2, in addition to its known pro-inflammatory action, can provide important anti-inflammatory roles, at least in some model systems [Gilroy D W, et al. (2003). FASEB 17:2269-71 and Gilroy D W, et al. (1999). Nat Med 5:698-701]. Therefore, the role of the cyclooxygenase-prostaglandin (COX-PG) axis in the pathology and management of TMJ arthritis and disorders can be examined.

a) Anti-Inflammatory Regimen in COLL1Pr-IL1β^{XAT} Transgenic Mice

It has been established that IL-1β drives the expression of COX-2 to form prostaglandin E₂ (PGE₂), a principal mediator of inflammation in a number of tissues, including joints [Agarwal S, et al. (2001) Arthr Rheum 44: 608-17; Yoshida H, et al. (2002) J Oral Rehab 29:1146-52; and Hutchins B, et al. (2002) J Orofac Pain 16:312-6]. COX-2, as well as the constitutively expressed COX-1, can be temporally (time course) and spatially (sites of expression) characterized at the molecular level, and can be correlated with PGE₂ levels and other inflammatory mediators related to arthritis, as well as neurotransmitter expression and behavioral measures in the IL-1β^{XAT} Tg mice. Tg mice of the founder line can be treated with a COX-2 selective inhibitor such as NS-398; [Kyrkanides S, Moore et al. (2002) Mol Brain Res 104: 159-69]). NS-398 can be administered to the mice via chow (125 ppm). In addition, the role of the constitutive COX-1 can be examined in the development of TMJ pathology and TMD by administering SC-560, a COX-1 inhibitor (64 ppm in chow), as well as a mixed inhibitor (ibuprofen at 375 ppm in chow). This route of administration simplifies long-term treatment (weeks-months) and the oral doses required for specifically inhibiting these enzymes in vivo have been previously determined [Jantzen P T, et al. (2002) J Neurosci 22:2246-54 and Mueller-Decker K, et al. (2002) J Invest Dermatol 119: 1189-95].

b) Experimental Design

As summarized in Table 5, below, TMJ inflammation can be induced in 8 weeks old COLL1-IL1β^{XAT} transgenic mice by intra-articular injection of FW(Cre) as described above. A lag time between FIV(Cre) injection and development of significant behavioral and/or histological changes is expected. In keeping with the clinical use of NSAIDs, anti-inflammatory treatment can be initiated at a time when the FIV(Cre)-injected mice begin to demonstrate TMJ pathology and dysfunction. Alternatively, anti-inflammatory treatment can begin at a set time before or after the FIV(Cre) injection.

The mice can be sacrificed at various time points following initiation of anti-inflammatory treatment (4-8-16-24 weeks). Consequently, the effects of therapy on TMJ arthritis (anatomic, histologic, molecular changes) and dysfunction (behavioral changes), as well as on central nervous system changes as described above can be characterized. Efficacy of drug therapy can be evaluated by measuring the levels of PGE₂ in TMJ extracts in experimental and control mice as previously described [O'Banion M K, et al. (1991) J Biol Chem 266: 23261-7 and O'Banion M K, et al. (1992) Proc Natl Acad Sci U.S.A. 89:4888-92].

c) Statistical Analysis

At each of the four time points, nonparametric ANOVA methods can be used to test whether behavioral measures (rubbing of face, head flinching and resistance to mouth opening), central nervous system changes (SP and CGRP) and peripheral inflammation differ across the treatment groups. Mice in the 24 week group can have EMG measurements taken at each of the 4 time points as well as initially at base line. EMG can be compared both across time and between treatment groups using a linear mixed model, where each mouse is a cluster, EMG is the response, and time and treatment group indicators are the covariates. The extent to which each outcome is associated with IL-1β levels using spearman's correlation as a description measure can be assessed, and then formally testing whether the slope is zero in a linear regression model.

TABLE 5
NSAIDs in the management of TMJ arthritis and dysfunction.

| GROUP | Drug | Time Points | Animals (N = 10) | STUDIES |
|---|---|---|---|---|
| FIV(Cre) | NS-398 | 4-8-16-24 wks | 10 × 4 time points = 40 | Behavioral studies |
| FIV(Cre) | SC-560 | 4-8-16-24 wks | 10 × 4 time points = 40 | Histology studies |
| FIV(Cre) | ibuprofen | 4-8-16-24 wks | 10 × 4 time points = 40 | Molecular studies |
| FIV(Cre) | — | 4-8-16-24 wks | 10 × 4 time points = 40 | |
| FIV(lacZ) | — | 4-8-16-24 wks | 10 × 4 time points = 40 | |
| | | | Total mice = 200 | |

The effects of non-steroidal anti-inflammatory treatment, including selective COX-1 and COX-2, and a mixed inhibitor, can be analyzed on TMJ arthritis and dysfunction in COLL1-IL1β^{XAT} transgenic mice injected intra-articularly with FIV(Cre). FIV(lacZ) injected animals can be included to provide baseline data. The mice can be studied at various time points after treatment; at each time point, ten mice can be sacrificed in order to evaluate TMJ and central nervous system changes.

d) The Role of COX-2 in the Development of IL-1β Induced Arthritis and TMD

To confirm the role of COX-2 in the development of TMJ arthritis and dysfunction, IL-1β^{XAT} Tg mice can be crossed with COX-2^{-/-} knockout mouse and the effects of the conditional induction of IL-1β investigated in the TMJ as described above. Male and female breeders for COX-1^{+/-} (002180-T) and COX-2^{+/-} (002181-T) heterozygous knockout mice can be purchased from Taconic Laboratories (Germantown, N.Y.), for example, and crossed with COLL1-IL1β^{XAT} transgenics twice to generate heterozygous COX and homozygous COLL1-IL1β^{XAT} transgenic animals. The desired genotype can be generated by back-crossing these mice to the COX-1^{+/-} and COX-2^{+/-} heterozygous mice. Genotyping for the COX-1 and COX-2 genes can be performed as follows. DNA can be extracted from tail clips using a Wizard DNA isolation kit (Promega). Genotype is established by PCR as follows.

For COX-1 genotyping, SEQ ID NO:1 5′AGGAGATGGCTGCTGAGTTGG3′ and SEQ ID NO:2 5′AATCTGACTTTCT GAGTTGCC3′ are used to detect the intact COX-1 exon 11; SEQ ID NO:3 5′GCAGCCTCTGTTCCACATACAC3′ and SEQ ID NO:4 5′AATCTGACTTTCTGAGTTGCC3′ are used to detect the disrupted COX-1 exon 11 containing the neomycin gene.

For COX-2 genotyping, SEQ ID NO: 1 5′ACACACTCTATCACTGGCAC3′ and SEQ ID NO:6 5′AGATTGTTGTCAGTATCTGCC3′ are used to detect the endogenous COX-2 gene (the PCR product extending from exon 8 to exon 10); SEQ ID NO:7 5′ACGCGTCACCTTAATATGCG3′ and SEQ ID NO:8 5′AGATTGTTGTCAGTATCTGCC3′ are used to detect the targeted disruption of COX-2 exon 8 containing the neomycin gene.

Using the aforementioned strategy, litters can be obtained comprised of COLL1-IL1βXAT transgenic animals, 25% COX null, 50% COX heterozygous, and 25% COX wildtype. The ability to test FIV(Cre) activation of the COLL1-IL1β^{XAT} transgene in littermates with differential expression of COX enzymes is critical since the background strains are mixed. As summarized in Table 6, COX-1^{-/-}/COLL1-IL1β^{XAT} and COX-2^{-/-}/COLL1-IL1β^{XAT} mice can be subjected to FW(Cre) intra-articular injection and induction of IL-1β in their TMJ at 8 weeks of age. Moreover, additional groups of mice receiving FIV(lacZ) can serve as controls. The mice can be sacrificed at specific time points post-treatment (4-8-16-24), and the development of TMJ arthritis (anatomic, histologic, molecular changes) and dysfunction (behavioral changes), as well as on central nervous system changes, can be investigated.

e) Statistical Analysis

The analysis here can be the same as described herein, except, for example, on could have eight groups and two treatments. The analyses can be carried out separately for each group. In brief, at each of the four time points, nonparametric ANOVA methods can be used to test whether behavioral measures (rubbing of face, head flinching and resistance to mouth opening), central nervous system changes (SP and CGRP) and peripheral inflammation differ across the treatment groups. Mice in the 24 week group can have EMG measurements taken at each of the 4 time points as well as initially at base line. EMG can be compared both across time and between treatment groups using a linear mixed model, where each mouse is a cluster, EMG is the response, and time and treatment group indicators are the covariates. In addition, one can assess the extent to which each outcome is associated with IL-1β levels using spearman's correlation as a description measure, and then formally testing whether the slope is zero in a linear regression model.

TABLE 6
The roles of COX-1 & COX-2 in the development of TMJ arthritis and dysfunction.

| GROUP | TREATMENT | Time Points | Animals (N) |
|---|---|---|---|
| COX-2^{-/-}/COLL1-IL1β^{XAT} | FIV(Cre) | 4-8-16-24 wks | 10 × 4 time points = 40 |
| COX-2^{+/-}/COLL1-IL1β^{XAT} | FIV(Cre) | 4-8-16-24 wks | 10 × 4 time points = 40 |
| COX-2^{+/+}/COLL1-IL1β^{XAT} | FIV(Cre) | 4-8-16-24 wks | 10 × 4 time points = 40 |
| COX-2^{+/+}/COLL1-IL1β^{XAT} | FIV(lacZ) | 4-8-16-24 wks | 10 × 4 time points = 40 |
| COX-1^{-/-}/COLL1-IL1β^{XAT} | FIV(Cre) | 4-8-16-24 wks | 10 × 4 time points = 40 |
| COX-1^{+/-}/COLL1-IL1β^{XAT} | FIV(Cre) | 4-8-16-24 wks | 10 × 4 time points = 40 |
| COX-1^{+/+}/COLL1-IL1β^{XAT} | FIV(Cre) | 4-8-16-24 wks | 10 × 4 time points = 40 |
| COX-1^{+/+}/COLL1-IL1β^{XAT} | FIV(lacZ) | 4-8-16-24 wks | 10 × 4 time points = 40 |
| | | | Total mice = 320 |

The role of COX-2, as well as of the constitutive COX-1, in temporomandibular joint disorders can be confirmed by inducing long-term expression of IL-1β in the TMJ of COX-1^{-/-} and COX-2^{-/-} knockout mice. To this end, COX-1^{-/-}/COLL1-IL1β^{XAT} and COX-2^{-/-}/COLL1-IL1β^{XAT} mice can be injected intra-articularly with FIV(Cre) and studied over time. In addition, COX heterozygous and wild-type mice can be included as controls. Mice (N = 10) can be sacrificed at various time points after treatment, in order to evaluate any TMJ and central nervous system changes.

### f) Anticipated Results

It is expected that anti-inflammatory therapy will attenuate nociception based on clinical data [Martel-Pelletier J, et al. (2003). Semin Arthritis Rheum 33:155-67] as well as animal studies [Yoshida H, et al. (2002). J Oral Rehab 29:1146-52 and Ochi T, et al. (2003). Biochem Pharmacol 66:1055-60]. However, the relative role of the inducible COX-2 and the constitutive COX-1 in the development of TMJ pathology and dysfunction remains unknown. In fact, previous reports on the subject provide conflicting evidence [Yoshida H, et al. (2002). J Oral Rehab 29:1146-52; Hutchins B, et al. (2002). J Orofac Pain 16:312-6; Jantzen P T, et al. (2002). J Neurosci 22:2246-54; Mueller-Decker K, et al. (2002). J Invest Dermatol 119: 1189-95; Martel-Pelletier J, et al. (2003). Semin Arthritis Rheum 33:155-67; and Ochi T, et al. (2003). Biochem Pharmacol 66:1055-60]. Therefore, it is important that both COX isoforms are included as disclosed herein and their roles addressed through the use of selective inhibitors as well as non-selective drugs. Another point of potential importance is that drugs targeting COX isoforms may lead to concomitant upregulation of the parallel 5-lipoxygenase pathway. Recent studies on the effectiveness of dual inhibitors of cyclooxygenase and 5-lipoxygenase (ML3000) suggest that simultaneous inhibition may be required to obtain adequate levels of anti-inflammatory action [Fiorucci S, et al. (2001) Biochem Pharmacol 62: 1433-8 and Jovanovic D V, et al. (2001). Arthr Rheumaton 44:2320-30]. In fact, such drugs are currently in Phase III clinical trials in Europe. These can also be tested in the disclosed mice and systems.

Interestingly, COX-1 [Zhu X, et al. (2003). Pain 104:15-23] and COX-2 [Yaksh T L, et al. (2001). J Neurosci 21:5847-53 and Choi H S, et al. (2003). Neurosci Letters 352: 187-90], in addition to their roles in peripheral inflammation, both appear to be involved to some degree in the central processing of pain at the level of the central nervous system. Overall, it is expected behavioral and pathological benefits from NS-398 administration and that this will also be confirmed with the COX-2 knockout mouse experiment, disclosed herein. In some model systems COX-1 has been found to influence inflammation and pain [Zhu X, et al. (2003). Pain 104:15-23 and Siqueira-Junior J M, et al. (2003). Pharmacol Res 48:437-43]. Recently, in addition to COX-1 and COX-2, at least two new PGE₂ synthase isoforms have been added to the family of enzymes that result in the production of prostaglandins: the membrane-associated mPGES, which is functionally coupled to COX-2, and the cytosolic cPGES that appears to be linked to COX-1 dependent PGE₂ production [Tanioka T, et al. (2000). J Biol Chem 275:32775-82 and Murakami M, et al. (2000). J Biol Chem 275:32783-92]. Although cellular localization may play some role, functional coupling is largely a factor of expression patterns: as with COX-2, mPGES is dramatically upregulated by proinflammatory stimuli, whereas cPGES is constitutively expressed in cell systems examined to date [Jakobsson P J, et al. (1999). Proc Natl Acad Sci USA 96:7220-25; Stichtenoth D O, et al. (2001). J. Immunol. (2001) 167:469-74; and Han R, et al. (2002). Biol Chem 277:163555-64]. In addition, COX-2 and mPGES are coordinately upregulated in a rat model of adjuvant arthritis [Lehmann, et al. (1997). J Biol Chem 272:3406-10]. Therefore, mPGES may play a role in our model of IL-1, induced arthritis, and one can investigate the regulation of mPGES as part of the proposed experiments. This can be readily accomplished by employing methods established and routinely used in our laboratories [Moore A H, et al. (2003). In Press]. Recently, a splice variant of COX-1 that retains intron 1 was described in canine brain and called COX-3 [Chandrasekharan N V, et al. (2002). Proc Natl Acad Sci USA. 99:13926-31]. Although this variant mRNA is found in rat and mouse brain, it does not appear to be regulated by inflammatory stimuli [SS, et al. (2003). Brain Res Mol Brain Res. 119: 213-5; K is B, Snipes J A, Isse T, Nagy K, Busija D W. (2003) J Cereb Blood Flow Metab. 23: 1287-92; and Dinchuk J E, Liu R Q, Trzaskos J M (2003) Immunol Lett. 86:121]. More importantly, retention of intron 1 in human and rat results in a frameshift leading to a nonsense transcript; therefore it is believed not to play any role in the production of prostanoids [K is B, Snipes J A, Isse T, Nagy K, Busija D W. (2003) J Cereb Blood Flow Metab. 23: 1287-92 and Dinchuk J E, Liu R Q, Trzaskos J M (2003) Immunol Lett. 86:121]. This also appears to be the case for mouse COX-3 [Shaftel S S, et al. Brain Res Mol Brain Res. 2003 Nov. 26; 119(2):213-5. Erratum in: Brain Res Mol Brain Res. 2004 Apr. 7; 123(1-2):136].

As discussed herein, the timing of anti-inflammatory therapy (NS-398, SC560 or ibuprofen in chow) in relation to FIV(Cre) injection may be critical. Disclosed herein is evidence that a non-selective inhibitor (indomethacin) can paradoxically increase expression of inflammation-related genes following IL-1 treatment (FIG. 7). Moreover, Gilroy et al. [Gilroy D W, et al. (1999). Nat Med 5:698-701] recently reported that COX-2, in addition to its known pro-inflammatory action, can also provide important anti-inflammatory roles, at least in some model systems. More specifically, if NSAID treatment is given prophylactically (prior to initiation of injury or inflammation), then it can exert significant anti-inflammatory effects [Kyrkanides S, Moore et al. (2002). Mol Brain Res 104: 159-69 and O'Banion M K (1999). Crit. Rev Neurobiol 13: 45-82]. In contrast, if the anti-inflammatory regimen is started after inflammation commences, the inflammatory response can be exacerbated by inhibiting COX-2-derived anti-inflammatory prostaglandins ([Gilroy D W, et al. (2003). FASEB 17:2269-71 and Gilroy D W, et al. (1999). Nat Med 5:698-701]; also see FIG. 7). In the clinical setting, anti-inflammatory drugs are most often taken after injury and the initiation of inflammation for pain alleviation. In order to replicate the clinical conditions as closely as possible, one can begin the anti-inflammatory treatment after the induction of IL-1β and establishment of pathology and pain in the mouse TMJ by FIV(Cre) injection. One can repeat the experiment with inhibitors started before FIV(Cre) injection.

### Example 3

### IL-1β Constructs and Mice Related Nerve Specific Expression

Using standard cloning techniques, including application of RT-PCR, the cDNA encoding mature human IL-1, (i.e. missing the pro-IL-1β sequences cleaved by caspase 1) was cloned in-frame with the heterologous signal sequence for human IL-1ra. This construct is described in more detail herein and was verified by DNA sequencing. This hybrid cDNA was inserted into an XAT universal vector harboring a CMV promoter and used to transfect human embryonic kidney cells (293H) together with either pRC/CMV (control) or the pRC/CMV expression vector harboring a wild type cre recombinase cDNA (pRC/CMV-cre). As shown in FIG. 11, the presence of cotransfected cre Recombinase leads to robust expression of the lacZ gene (here detected by X-gal histochemistry), which is downstream of the modified IL-1β cDNA and separated by an IRES element. The relative lack of lacZ activity in control cultures transfected in parallel (left panel) indicates that the construct undergoes recombination and transcriptional activation in the presence of cre recombinase. The human IL-1ra cDNA was also cloned into the same XAT vector, and the sequence confirmed.

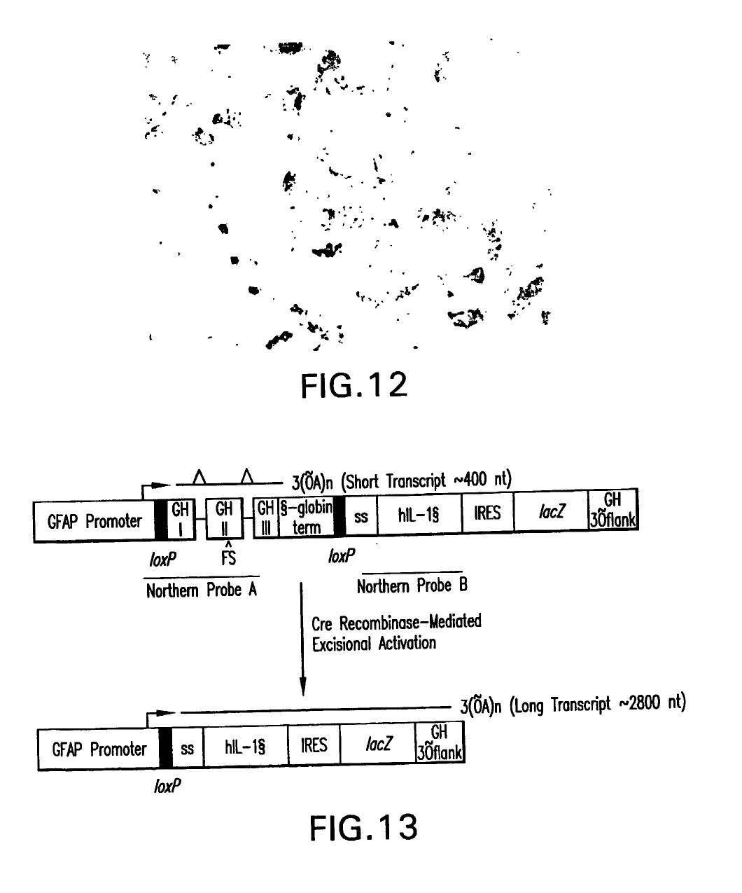

In order to demonstrate the feasibility of using the pseudotyped Feline Immunodeficiency Virus system for transduction of murine astrocytes, infections were carried out with FIV harboring lacZ in primary cultures of astrocytes established from postnatal day one mice [O'Banion, M. K., et al. Neurochem. (1996) 66:2532-2540]. As seen in FIG. 12, lacZ was readily transduced in these cells as evidenced by X-gal histochemistry. Cultures transduced with an FIV vector lacking lacZ showed no staining.

a) Engineering the IL-1β^{XAT} Construct (See FIG. 13)

The IL-1 excisional activation transgene (IL-1 XAT) is designed to be transcriptionally active in astrocytes by virtue of a glial fibrillary acidic protein (GFAP) promoter; be incapable of producing IL-1 until cre recombinase removes an inactivating cassette; and upon activation, produce a secreted and active hIL-1β that does not depend on IL-1 cleaving enzyme (ICE; caspase-1) activity as well as co-express lacZ that can be assayed at the cellular level. This construct can be derived from an available cassette [Brooks, A. I., et al. Nature Biotech. (1997) 15:57-62] modified by replacement of the NSE promoter with the CMV promoter by: 1) substituting the CMV promoter with a human GFAP promoter to provide astrocyte specific expression in mice [Brenner, M., et al. J. Neurosci. (1994) 14:1030-1037], and 2) inserting a modified hIL-1β cDNA that has a heterologous signal sequence (derived from IL-1ra) and mature IL-1β coding sequence. Although microglia are a major source of IL-1 in the brain, the GFAP astrocyte-specific promoter represents the only well-characterized glial promoter in transgenic mice. The modified hIL-1β sequence was chosen on the basis of a high rate of IL-1β secretion and demonstrated activity in mice [Gjörloff-Wingren, A., et al. (1996) Cell. Immunol. 169:226-237; Björkdahl, O., P. et al. (1999) Immunology 96:128-137]. Use of a human IL-1β also provides a means to distinguish transgene expression from endogenous mouse expression since species-specific probes and antibodies are available.

b) Ex Vivo Testing of the IL-1β^{XAT} Construct

Prior to generating transgenic mice, the functionality of the IL-1 XAT construct can be verified.

a) To test the functionality of the loxP elements, the IL-1 XAT construct can be transformed into an E. coli strain that constitutively expresses cre recombinase. Southern blot analysis of independent transformants from cre-expressing and cre-non-expressing bacteria can be performed to determine the efficiency of loxP-mediated recombination.

b) To verify that hIL-1β is produced following recombination, co-transfection of IL-1 XAT and CMV-cre recombinase can be performed in an established rat astrocyte cell line that shows high level GFAP expression (RBA cells) [Kimmich, G. A., et al. (2001) J. Membr. Biol. 182:17-30]. Recombination in astrocytes can be detected by utilizing PCR primers that flank the inactivating cassette. Transgene transcription can be monitored by RT-PCR for the IL-1β transcript, ELISA for hIL-1β, and biochemical or histochemical detection of lacZ expression. The specificity of the GFAP promoter can be ascertained by carrying out a comparable analysis in a mouse fibroblast cell line (NIH3T3 cells). In this case it was anticipate detecting recombination, but not observing transgene expression. Transfections in primary cultures of mouse astrocytes can also be carried out.

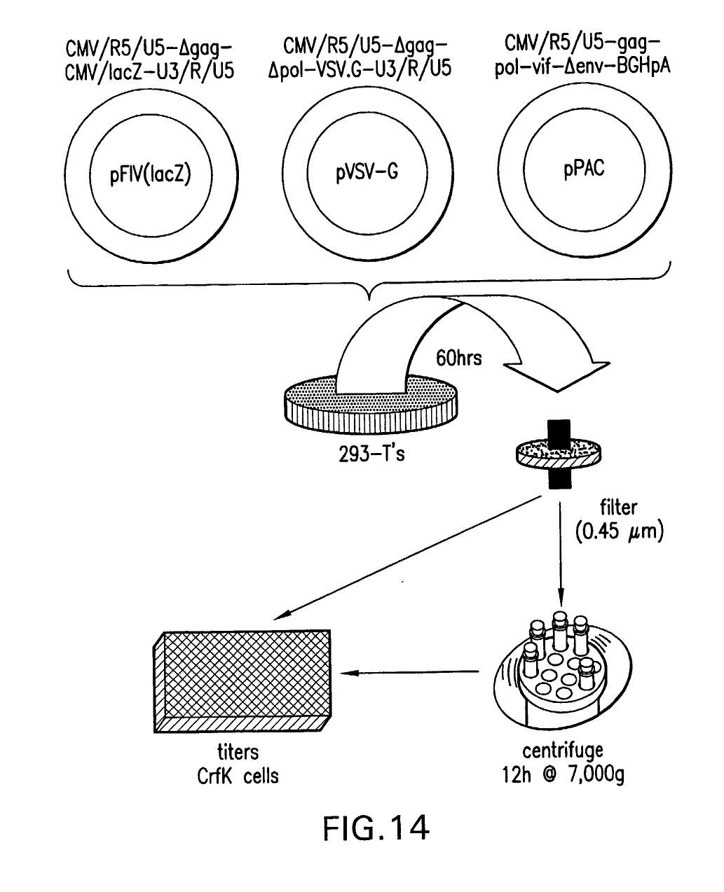

c) To demonstrate that the hIL-1β is biologically active, co-transfections of IL-1 XAT and CMV-cre recombinase can be carried out in the rat astrocyte cell line and supernatants collected over 72 hours. Conditioned media can be placed on cultures of mouse astrocytes. Total RNA can be harvested after 4 h and levels of COX-2 mRNA can be quantified by real-time RT-PCR. It is believed that COX-2 is a very sensitive indicator for IL-1β activity [O'Banion, M. K., et al. (1996) Neurochem. 66:2532-2540 and Kyrkanides, S., et al. (1999) J. Neuroimmunol. 95:95-1076]. Specificity can be established by using a neutralizing antibody directed to human IL-1β (R & D Systems). Although rat IL-1β is less potent for the mouse IL-1 type 1 receptor than mouse IL-1β [Liu, C., Y. Bai, et al. J. Interferon Cytokine Res. (1995) 15:985-992], the assay may be confounded by rat astrocyte products. Supernatants from cells transfected with the pRC/CMV vector alone should help control for this problem. An alternative approach is to create stable cell lines harboring IL-1 XAT by cotransfection with a neomycin resistance marker plasmid and selection in G418. Stable lines can be transduced with FIV-cre (See FIG. 14) to produce large amounts of hIL-1β. In all cases, control supernatants can be collected from cells transfected or transduced using a vector that lacks cre recombinase.

A similar strategy can be employed for examining production of IL-1ra from the IL-1ra XAT construct. Again, recombination and gene expression would be followed by PCR and X-gal histochemistry and product secretion would be ascertained by ELISA. For the activity assay, murine astrocytes would be treated with conditioned media (containing IL-1ra) and concentrations of recombinant murine IL-1β that are sufficient to elicit a detectable COX-2 response. Supernatants from control cultures and the use of a neutralizing antibody to human IL-1ra (R & D Systems) can be required to confirm specificity.

c) IL-1β and IL-1ra Excisional Activation Transgenic (XAT) Mouse Lines and Test for Functional Recombination and Expression of Transgenes Following Viral Transduction

The XAT constructs can be excised from their bacterial vectors and injected into the male pronucleus of fertilized eggs to generate multiple lines of transgenic mice. Founders can be identified by PCR screening and confirmed with Southern blot analysis. All aspects of transgene introduction and founder husbandry can be carried out using standard techniques. Transgenes can be introduced on a pure C57B1/6 line. Previous experience suggests that 4 to 6 lines can be identified for each transgene. Founder lineages can be analyzed for transgene expression and recombinational activation.

IL-1β (and IL-1ra) XAT transgenic mouse lines can be generated and tested for functional recombination and expression of transgenes following viral transduction. The IL-1 XAT construct can be excised from its bacterial vector and injected into the male pronucleus of fertilized eggs to generate multiple lines of transgenic mice. Founders can be identified by PCR screening and confirmed with Southern blot analysis. All aspects of transgene introduction and founder husbandry can be carried out using standard techniques. Transgenes can be introduced on a pure C57B1/6 line. Previous experience suggests that 4 to 6 lines can be identified for each transgene. Founder lineages can be analyzed for transgene expression and recombinational activation.

a) To identify lines showing active GFAP transgene promoter utilization, Northern blots of transgenic mouse brain RNA can be carried out using a probe homologous to the short, inactive transcript (GFAP 5′ UTR and GH) predicted to be synthesized from the GFAP promoter (see FIG. 13). Once lines are identified, combined in situ hybridization and immunocytochemistry can be used to verify colocalization of the short transcript and endogenous GFAP protein.

b) To demonstrate excisional activation in vitro, primary cultures of astrocytes can be established from transgenic neonatal brain. Astrocytes from transgenic and control non-transgenic animals can be infected with either FIV-cregfp (FIG. 14) or the FIV-gfp control virus. PCR can be used to assay cre-induced recombination of the IL-1 XAT transgene, and transgene activation can be monitored by ELISA assay for hIL-β and measures of lacZ activity. Efficiency of cre-mediated excisional activation can also be assessed by measuring the ratio of lacZ⁺ cells to GFP⁺ cells by X-gal histochemistry and GFP epifluorescence. Viral vector stocks can be prepared and titered using established and routine methods.

c) To demonstrate excisional activation in vivo, adult IL-1 XAT transgenic mice can be injected with FIV-cregfp or the control virus, FIV-gfp (see below), in the frontal cortex (bregma: 0.5 mm, lateral 1.8 mm, depth 1.8 mm relative to the skull surface) using a microprocessor controlled, 33-gauge needle and slow delivery rate (100 nl/min over a 10 min period). This method provides localized and reproducible administration of activating virus to the brain, with minimal intraventricular or contralateral effects [Brooks, A. I., et al. J. Neurosci. Meth. (1998) 80:137-147]. Moreover, the small needle track and slow infusion rate minimize tissue reaction to needle injury, a potentially confounding variable in the proposed studies of IL-1 expression. Two weeks following injection, animals can be subjected to analysis for: 1) DNA recombination by PCR amplification of DNA extracted from the injection site using primer pairs that flank the loxP elements, and 2) activation of transgene expression. This later analysis can include in situ hybridization for IL-1β expression combined with immunocytochemistry for endogenous GFAP and tissue ELISA for human IL-1β levels. In addition, X-gal histochemistry for lacZ expression can be carried out with every fifth section from a minimum of 4 transgenic animals receiving FIV-cregfp. In this way, the extent of transgene activation and the variability between animals can be established. These measures can help establish the number of animals required for future studies examining interactions with other transgenes (i.e. Aim 3) or with injury paradigms. 8 animals can be used from each transgenic line for these initial studies. Six animals can be injected unilaterally with FIV-cregfp to induce recombination and 2 animals can be injected unilaterally with the control viral vector. Two animals from the FIV-cregfp can be sacrificed and the region surrounding the injection site can be dissected and subject to PCR analysis for DNA recombination and hIL-1β expression. Murine IL-1β and COX-2 levels can also be measured by real-time RT-PCR. Control tissue can be derived from the contralateral hemisphere. The remaining animals (FIV-cregfp plus the control injections) can be prepared for histological investigation as described above.

(1) Feline Immunodeficiency Viral (FIV) Vectors

For activation of the silent transgenes disclosed herein a VSV-G pseudotyped Feline immunodeficiency Virus system developed by Poeschla et al. (1998) can be employed. This lentivirus has been shown to efficiently infect dividing, growth arrested as well as post-mitotic cells. Furthermore, it allows for incorporation of the transgene into the host's genome, leading to stable gene expression. This is a 3-vector system, whereby each confers distinct instructions: the FIV vector carries the transgene of interest and lentiviral apparatus with mutated packaging and envelope genes. A vesicular stomatitis virus G-glycoprotein vector (VSV-G; [Bums, J. C., et al. Proc. Natl. Acad. Sci. USA 90:8033-8037]) contributes to the formation of the viral envelope in trans. The third vector confers packaging instructions in trans [Poeschla, E. M., et al. (1998) Nature Med. 4:354-357].

(2) FIV Production and Concentration

Cultured 293-T cells are transfected with a FIV DNA cocktail (20 μg of pFIV, 15 μg of pVSV-G and 5 μg of pPAC) using the Lipofectamine 2000 reagent per manufacturer's instructions (Invitrogen). Sixty hours later, the supernatant is collected and filtered (0.45 μm). This FIV-rich solution can be used directly or further concentrated to increase titers. The concentration process is based on an overnight centrifugation of FIV solution at 7,000×g at 4° C. using a Sorvall RC 5B plus centrifuge with a SS-34 rotor. The supernatant is then decanted and the viral pellet is reconstituted in sterile saline with 40 mg/ml lactose. Titers are established on feline kidney CrfK cells (ATCC) by counting blue forming units after X-gal histochemistry, and routinely range 10⁷-10⁸.

(3) Viral Infection

Mice are anesthetized with Isofurane (2.5% in O₂) and placed in a stereotaxic instrument. Prior to surgery mice can be placed on a Gaymar, thermostat controlled, water blanket. A rectal thermocouple is used for body temperature control. Surgical plane of anesthesia can be assessed using a tail/toe pinch reflex and corneal reflex. A stereotaxic injection is performed using a frame mounted micromanipulator holding a Hamilton syringe and 33 GA needle. The microsyringe is mounted in a Micro-1 microsyringe pump controller (World Precision Instruments) that allows for a continuous injection over a controlled time. 1.5 μl volumes of FIV-gfp or FIV-cregfp can be injected into mouse frontal cortex using the following coordinates: bregma: 0.5 mm, lateral 1.8 mm, depth 1.8 mm relative to the skull surface. Injections can be performed over a time interval of not less than 10 minutes to prevent any possible pressure backflow of the solution and to minimize nerve cell injury around the needle tract. After completion of the injection, the needle is slowly raised over 3-5 min and the burr hole can be covered with Ethicon bone wax. The soft tissues of the scalp can be sutured using 6-0 Ethicon monofilament nylon.

(4) Quantification of mRNA Abundance by Real-Time RT-PCR

Cortex containing the injection site can be dissected out and frozen in isopentane chilled with dry ice. The tissue can be stored in sterile tubes at −80° C. until ready for RNA isolation. RNA can be isolated using Trizol reagent (Invitrogen), precipitated and the concentration determined by spectrophotometry. First-strand DNA can be synthesized by using 2 μg of DNase-treated RNA, oligo(dT) primers, and Superscript II (Invitrogen) according to the manufacturer's instructions.

Quantification of mRNA levels can be carried out using an iCycler (Bio-Rad) and real time PCR with SYBR Green as the fluorescent marker (Molecular Probes). Prior to PCR of the cDNA samples, PCR conditions can be optimized for each mRNA to be analyzed. Standard curve reactions can be performed by varying annealing temperatures, Mg²⁺, primer, and SYBR green concentrations. Melt curve analysis can be also completed for each PCR amplification to confirm production of a single product with the expected melting temperature. Serial dilution of the starting cDNA template can demonstrate linear amplification over at least 5 orders of magnitude. Using the iCycler IQ 2.3 software (Bio-Rad) to analyze efficiency, PCR conditions can be varied until an efficiency of at least 95% is obtained. PCR reactions can be performed in a volume of 25 μl and typically contain 4.0 mM Mg²⁺, 0.2 μM concentrations of each primer, 1 μl of SYBR green (1:100,000 final dilution), 100 μM nucleotide mix (Stratagene), 0.5 U of Platinum Taq in PCR buffer (Invitrogen), and 1 μl of cDNA sample. To ensure consistency, a master mix can first be prepared containing all reagents except the cDNA sample. The primers were designed using the Oligo 6.0 program (Molecular Biology Insights, Inc., Cascade, Colo.) and are listed in the following table 7. In general, PCR reaction conditions can be the following: denaturation at 95° C. for 3 min, followed by 40 cycles of amplification by denaturing at 95° C. for 30 s, annealing at 64° C. for 30 s and extension at 72° C. for 60 s. PCR products can be monitored using SYBR Green fluorescence during the last 10 s of each extension step. Results can be expressed as the number of cycles to reach threshold. For each PCR run, selected samples can be serially diluted and then amplified in order to determine PCR efficiency. To correct for variations in starting RNA values, the level of G3PDH mRNA can be determined for each sample and used to normalize all subsequent mRNA determinations. Each PCR run can be completed with a melt curve analysis to ensure quantification of a single specific product.

TABLE 7
PCR Primers.

| | | |
|---|---|---|
| Molecule | Upper Primer | Lower Primer |

| | | |
|---|---|---|
| Mature | SEQ ID NO:9 | SEQ ID NO:10 |
| hIL-1β | GCACCTGTACGA | CTTTAGGAAGAC |
| | TCACTGAACTGC | ACAAATTGCA |
| | | TGG |
| Human | SEQ ID NO:11 | SEQ ID NO:12 |
| ssIL-1β | ATGGAAATCTGC | CTTTAGGAAGAC |
| | AGAGGCCTCC | ACAAATTGCA |
| | | TGG |
| Secreted | SEQ ID NO:13 | SEQ ID NO:14 |
| hIL-1ra | ATGGAAATCTGC | CTACTCGTCCTC |
| | AGAGGCCTCC | CTGGAAGTAGAA |
| | | TTTG |
| Lac Z | SEQ ID NO:15 | SEQ ID NO:16 |
| | TTTTTCCAGTTC | TTTATCGCCAAT |
| | CGTTTATCC | CCACATCT |
| Murine | SEQ ID NO:17 | SEQ ID NO:18 |
| IL-1β | GAGAACCAAGCA | GCATTAGAAACA |
| | ACGACAAAATAC | GTCCAGCCCA |
| | | TAC |
| Murine | SEQ ID NO:19 | SEQ ID NO:20 |
| COX-2 | CCGTGGGGAATG | CCAGGTCCTCGC |
| | TATGAGGA | TTATGATCTG |
| Murine | SEQ ID NO:21 | SEQ ID NO:22 |
| G3PDH | AGCACAGTCCAT | TCCACCACCCTG |
| | GCCATCAG | TTGCTGTA |

(5) Protein Quantification

ELISA kits for human IL-1β and human TL-1ra are obtained from R & D systems. For cell culture experiments, supernatants can be used directly or diluted with ELISA buffer as needed for assay. For measurements of cytokine in brain tissue, the area of interest can be carefully dissected and then homogenized in phosphate buffered saline (pH 7.4; 100 mg/ml) containing a protease inhibitor cocktail (Roche) at 4° C. Following centrifugation for 15 min at 8,000×g, supernatants can be collected and kept frozen in Eppendorf tubes at −80° C. All measurements can be related to total protein levels, determined using the micro-BCA method (Pierce).

(6) Immunocytochemistry

For immunocytochemistry of glial cells and Aβ deposits, mice can be anesthetized with IP ketamine (60-90 mg/kg) plus IP xylazine (4-8 mg/kg) and sacrificed by intracardiac perfusion with 4% paraformaldehyde in a sodium phosphate buffer, pH 7.2. The perfusion pressure is monitored to insure that it does not exceed 90 mm/Hg and artificially open the BBB. The brain can be removed and postfixed for 2 h. At this point brains can be coded to insure unbiased processing and analysis. Following equilibration with 30% sucrose in phosphate buffer, brains can be frozen, and 30 μm frozen sections cut on a sliding knife microtome. The sections can be stored in cryoprotective solution until ready for ICC processing. Sections can be processed using a free-floating method for immunocytochemical localization of GFAP (rabbit polyclonal; 1:2000 dilution, Dako), Mac-1 (monoclonal; 1:250; Serotec), MHC-II (monoclonal; 1:1000; Bachem), Aβ (rabbit polyclonal; 1:1000; BioSource #44-136), and phospho-tau (AT8; 1:500; Pierce # MN1020B). Visualization of all antibody-positive cells can be carried out by the Elite avidin-biotin (Vector Labs) procedure. Sections for Aβ ICC can be treated with 70% formic acid for 3 min prior to immunostaining. After extensive washing of the tissues and blocking of endogenous peroxidase by 30 min incubation in methanol containing 0.5% H₂O₂, the sections can be incubated in 10% normal goat serum for 1 h in PBS. The tissue can then be incubated 24-48 hours at 4° C. in the primary antiserum at the dilutions listed above in PBS containing 1% normal serum and 0.4% triton X-100. After extensive washing, the sections can be incubated in a biotinylated secondary antiserum for 2 hours. Subsequently, sections can be rinsed in PBS and incubated with the Elite avidin-biotin complex for 2 hours. After a final series of washes in a sodium acetate+imidazole buffer, the peroxidase reaction can be developed in a solution containing 0.05%, 3,3-diaminobenzidine (DAB), 0.1 M nickel sulfate, 0.125 M sodium acetate, 10 mM imidazole and 0.03% hydrogen peroxide. The reaction can be monitored visually and terminated with washes in PBS. X-gal histochemistry is accomplished by standard procedures [Olschowka, J. A., et al. Mol. Therapy. (2003) 7:218-227]. For double labeling of Aβ and activated glia, nickel can be omitted from the DAB reaction for Aβ (first reaction) to give a brown colored product. The ICC protocol can then be repeated for glial staining using nickel enhanced DAB or Vector Blue chromophore. Sections in which cell numbers or staining intensity can be compared between treatments can be processed together to limit variability. Sections can then be mounted, dehydrated and cover slipped with DPX. Control sections for antibody specificity can be processed simultaneously and can include incubations with normal serum in lieu of the primary antibody.

(7) Studies Examining Effects of Transgene Induction in Double Transgenic IL-1β (and IL-1ra) XAT/APPsw Mice

Heterozygous XAT mice from lines showing robust transgene induction can be crossed with heterozygous APPsw mice to generate double transgenic mice. Viral transduction can be carried out at 3 months of age and animals examined histologically at one and six months following viral transduction to determine the effects of transgene expression on glial activation, Aβ deposition, and tau phosphorylation. These studies can also be establish whether transgenes remain activated for a chronic period (6 months). Wild type, and XAT and APPsw single transgenic mice arising from the breeding strategy can be used as controls for these experiments.

(8) Double Transgenic IL-1β (and IL-1ra) XAT/APPsw Mice

Heterozygous XAT mice from lines showing robust transgene induction in Specific Aim 2 can be crossed with heterozygous APPsw mice to generate double transgenic mice. Viral transduction can be carried out at 3 months of age and animals examined histologically at three and nine months following viral transduction to determine effects of transgene induction on glial activation, Aβ deposition, and tau phosphorylation. These studies can also establish whether transgenes remain activated for a chronic period (9 months). Wild type, and XAT and APPsw single transgenic mice arising from the breeding strategy can be used as controls for these experiments.

All studies can be carried out using histological preparations. A total of 120 animals can be used in this experiment as summarized in Table 8, below, with 5 animals in each group. Parameters to be measured include localization/extent of transgene expression/viral transduction and the relationship of transgene expression to glial activation, Aβ deposition, and immunohistochemical evidence of tau phosphorylation. Fixed tissue can be sectioned in the coronal plane and used for quantitative and morphometric analyses. Localization of transgene expression can be accomplished by LacZ immunohistochemistry (cells that underwent recombination), detection of green fluorescent protein (for viral transduction), and antibodies specific for human IL-β or IL-1ra. If cytokine antibodies are not of sufficient sensitivity, sections can be subjected to in situ hybridization with probes for hIL-1β or hIL-1ra. Data to be gathered include regional density (i.e. in the vicinity of transgene expression versus a similar region in the adjacent contralateral hemisphere) of activated microglia and astrocytes (sections stained with Mac-1 and GFAP, respectively), numbers of activated microglia and astrocytes associated with Aβ deposits (using double ICC), and measures of amyloid deposition including total plaque burden (area covered), density, and size distribution of plaques (labeled by ICC). One of the most relevant measures of this relationship can be the number of activated microglia and astrocytes associated with Aβ plaques. This relationship appears to be influenced by NSAIDs in human brain tissue [Mackenzie, I. R. A. and D. G. Munoz. Neurology (1998) 50:986-990]. Data can be examined as a function of plaque size since numbers of activated microglia are highly correlated with extent of Aβ deposition [Frautschy, S. A, et al. Am. J. Pathol. (1998) 152:307-317]. In addition to comparisons between defined regions in control and activated mice, the local influence of transgene expression can be compared with adjacent tissue at specified distances from transduced cells using a zonal image analysis paradigm.

(9) Analysis of Glial Activation and Aβ Deposition

Stained sections can be viewed in a Zeiss Axioplan light microscope equipped with a DAGE color video camera, SONY high resolution color monitor, Apple Macintosh G4 computer, and Ludl X Y and Z motorized stage. Morphometric data can be collected using the stereological program NeuroZoom and its optical dissector method for unbiased cell counting. Areas containing cells to be counted can be determined using standard point counting grids. For the FIV induction studies, it is anticipated that this area can correspond to a 0.5 mm sphere surrounding the injection site. The extent of viral transduction by X-gal histochemistry, GFP immunofluorescence, and cytokine detection can be verified by antibody or in situ hybridization. Horizontal sections can be used and every fifth section can be counted initially until a power analysis can be completed to determine when significance is reached. The estimated number of labeled cells can be expressed as # per unit volume of cortex.

For analysis of transgenic mice, coronal sections can be used to obtain counts of activated glia and plaques in mouse cortex. Morphometric data (size of glia and plaques) can also be obtained from these sections. Sections can be sampled throughout the injection site and can be compared to a similar area in the contralateral hemisphere. Plaque size and density can be determined in the same cortical and hippocampal areas in every fifth section. Measures of activated glia associated with plaques can be obtained in two sets of five sections double stained for Aβ and GFAP or Aβ and MHC-II, respectively. For this analysis, plaque size can first be recorded by measuring the extent of brown staining. The number of activated glia can then be manually determined in an area extending 3 plaque radii from the center of the plaque. Small diffuse plaques and satellite plaques can not be included in this determination.

(10) Transgenic Mouse Genotyping

Mice can be tail clipped and ear punched at the time of weaning for genotyping and identification. DNA can be extracted from tail clips using a Wizard DNA isolation kit (Promega), which is a fast and highly reproducible method. Primers for detection of modified human IL-1β and human IL-1ra in transgenic mice are described below (under RT-PCR). APPtg2576 K/M670/1N/L (APPsw) can be maintained as hemizygotes on their C57BL/6/SJL background. To detect the PrP-APP transgene in the APPsw mice, DNA can be amplified using PCR with the primers SEQ ID NO:23 5′-CTGACCACTCGACCAGGTTCTGGGT-3′ (upper) and SEQ ID NO:24 5′-GTGGATAACCCCTCCCCCAGCCTAGACAA-3′ (lower). Use of this later primer with SEQ ID NO:25 5′-AAGCGGCCAAAGCCTGGAGGGTGGAACA-3′ amplifies part of the mouse PrP gene and can be used as a positive control in all genomic analyses [Hsiao, K., P. Chapman, S, Nilsen, C. Eckman, Y. Harigaya, S. Younkin, F. Yang and G. Cole. Science (1996) 274:99-102].

TABLE 8
Experimental groups to be examined at 9 months.^{a}

| Group | Genotype | Treatment | Number |
|---|---|---|---|
| 1 | Wild-type | FIV-gfp | 5 |
| 2 | Wild-type | FIV-gfpcre | 5 |
| 3 | APPsw | FIV-gfp | 5 |
| 4 | APPsw | FIV-gfpcre | 5 |
| 5 | hIL-1β XAT | FIV-gfp | 5 |
| 6 | hIL-1β XAT | FIV-gfpcre | 5 |
| 7 | hIL-1ra XAT | FIV-gfp | 5 |
| 8 | hIL-1ra XAT | FIV-gfpcre | 5 |
| 9 | APP sw X hIL-1β XAT | FIV-gfp | 5 |
| 10 | APP sw X hIL-1β XAT | FIV-gfpcre | 5 |
| 11 | APP sw X hIL-1ra XAT | FIV-gfp | 5 |
| 12 | APP sw X hIL-1ra XAT | FIV-gfpcre | 5 |

^{a}An identical set of animals would be established for a three-month trial

The APPsw mice are on a mixed background (C57B1/6×SJL) and are not viable on a pure C57 background. Because the C57B1/6 background is identical for the two transgenic XAT lines, it is anticipated that control wt mice and single transgenic APPsw mice arising from heterozygous crosses can show the same phenotype.

### Example 4

### IL-1β^{XAT} and RAP^{XAT} for Brain Expression Using the GFAP Promoter and Joint Expression using the Coll1 Promoter

a) Cloning of the Backbone IL-1β^{XAT} and RAP^{XAT} Vectors

The construct ssIL-1 beta (539 bp) codes for the signal sequence of the human interleukin-1 receptor antagonist (hIL-1RA, 75 bp) fused to the mature form of the human interleukin-1 protein [Wingren, A. G., et al., Cell Immunol, 1996 169(2):226-37]. ssIL-1 beta was amplified using standard PCR from human cDNA (obtained from the human monocytic cell line, U937) using the IL1B-17 kD-UP and IL-1B-17 kD-LP primers. The signal sequence from IL-1RA was added using 3 new upper primers that extended from the 5 prime end of the IL-1 beta mature product, and the lower primer from above. After each PCR reaction, the product was re-amplified with the next set of primers. The primers used were: IL1B-ss-UP2, IL1B-ss-UP3, IL1B-ss-UP4. The product from the last set of PCR primers was gel isolated and cloned into the vector pCRII-TOPO following the manufacturers protocol (Invitrogen). The resulting vector was transformed into E. Coli, and plasmid DNA was isolated from a single colony. Insert size and orientation was confirmed via restriction digests (EcoRI, HindIII, KpnI). Sequencing from the M13 and T7 primers within pCRII-TOPO identified clones with a nearly correct sequence, albeit in the opposite desired orientation. Errors at the 5 prime end near the ATG start of the ssIL-1 beta construct were corrected by reamplification of the construct using the upper primer HIL-1B-FIXUP, and the lower primer IL-1B-17 kD-LP, followed by re-cloning in pCRII-TOPO.

The construct hsIL-1RA (534 bp, also known as RAP) consists of the cDNA from the human secreted form of the IL-1 receptor antagonist, complete with its own signal sequence. This was amplified from human monocytic cDNA (cell line U937) using the HIL-1B-FIXUP upper primer and HSIL-1RA-LP lower primer. The product was cloned into pCRII-TOPO, and sequenced as described above for ssIL-1 beta.

ssIL-1 beta and hsIL-1RA were then sub-cloned into the commercial vector pBSII KS+ (Stratagene) using the EcoRI sites flanking the construct in pCRII-TOPO, and the single EcoRI site in pBSII KS+. This was done is order to reverse the construct orientations in pCRII-TOPO. Correct orientation and size in pBSII KS+ was confirmed using restriction digests (Eco RI, HindIII, KpnI). Sequences were confirmed using the T3 and T7 primers of pBSII KS+ (note hsIL-1RA contains a single base pair silent mutation).

The vector pBigT/CMV was originally derived as follows. The CMV promoter sequence was amplified from the pRc/CMV vector (Invitrogen, Carlsbad Calif.) using primers that included the PacI restriction enzyme cutting sites:

314. Upper primer:

(SEQ ID NO:73)

AAT ATC TTA ATT AAA TCT CTA GAT GCT TCG CGA TGT
ACG GGC
315. Lower primer:

(SEQ ID NO:74)

TAG TCA TAT ATG ATC TTA ATT AAA AGC TTG GGT CTC CC

The Pac I-flanked CMV construct was digested with Pac I, gel purified and subsequently cloned into the Pac I site of the pBigT vector upstream of a Lox P flanked (floxed) transcriptional termination cassette [Srinivas, S., et al., BMC Dev Biol, 2001 1(1):4; see website: www.srinivas.org for plasmid map and sequence]. The DNA sequences IRES-LacZ-Poly A were sub cloned from the vector pBSIRES-LacZ (described in PCT/US03/13672 which is herein incorporated by reference at least for material related to vector production) into pBigT/CMV using the unique XhoI and NotI sites within each of these vectors. The resulting vector was confirmed with Xho I restriction digestion, yielding a ˜11 kb sequence.

The constructs ssIL-1 beta and hsIL-1RA were subcloned from pBSII KS+ in the same manner as follows: The BamHI sites of the constructs in pBSII KS+ and the NheI site of pBigT/CMV were blunt ended using T4 DNA Polymerase. Next, all products were Sal I digested, and the constructs and resulting vector backbone ligated. The predicted final vector was confirmed via EcoRI digestion, yielding bands at ˜0.6, 2.7, 3.5 and 5 kbp. Final products—ssIL-1 beta in pBigT/CMV equals IL-1β^{XAT}, hsIL-1RA in pBigT/CMV=RAP^{XAT}.

b) Creation of GFAP-IL-1β^{XAT}, GFAP-RAP^{XAT}, and Mice Harboring these Transgenes

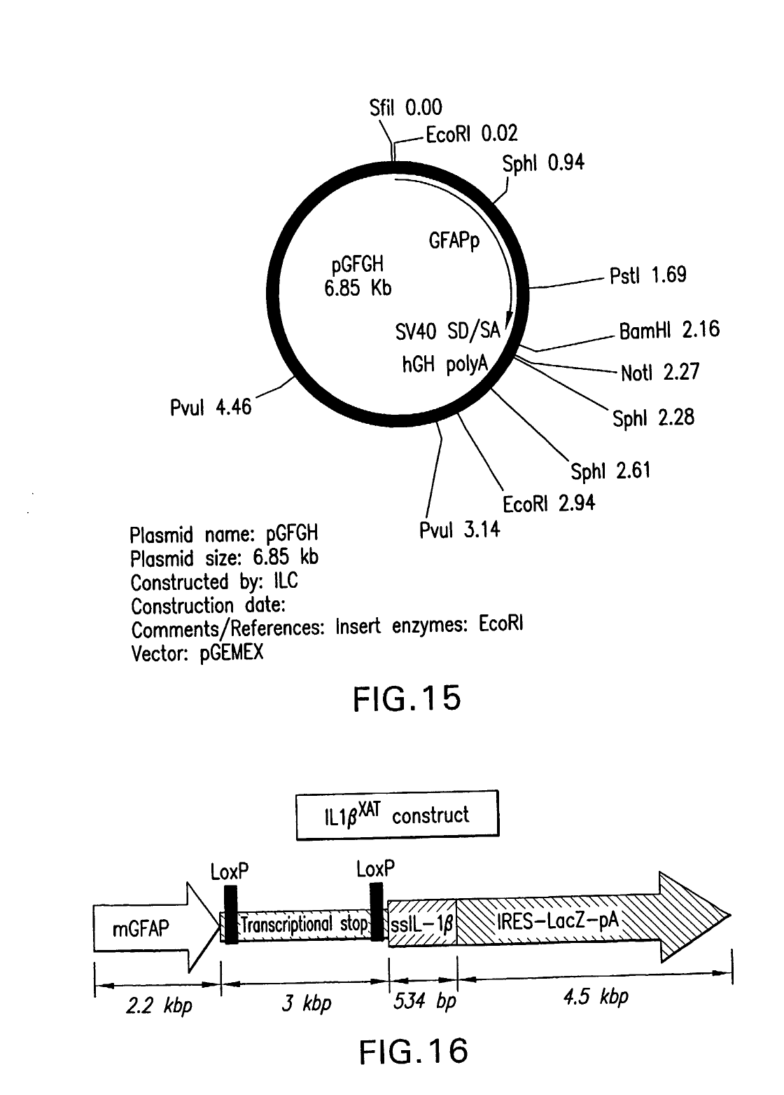

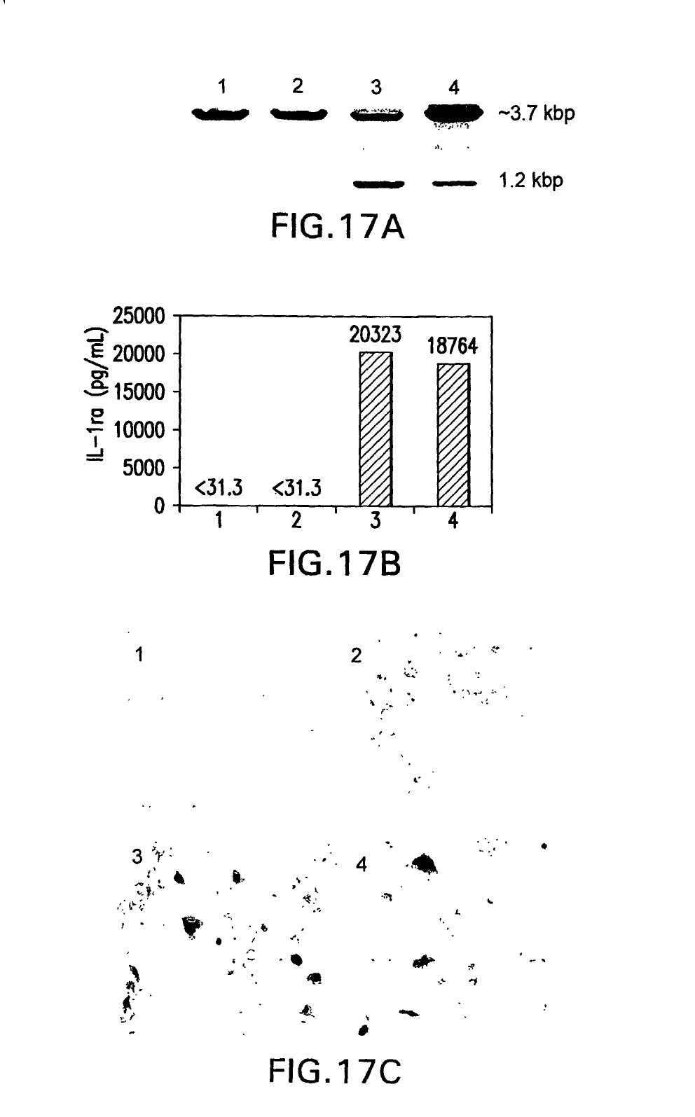

The final cloning step involved the substitution of the CMV promoter at the unique PacI site of pBigT with a murine glial fibrillary acidic protein (GFAP) promoter excised at EcoRI and NotI sites in the plasmid pGFGH (obtained from Ian Campbell at the Scripps Institute) and illustrated in FIG. 15). This promoter ensures neural cell (astrocyte) specific expression of the transgenes [Campbell, I. L., et al., Proc Natl Acad Sci USA, 1993 90(21):10061-5; Stalder, A. K., et al., Am J Pathol, 1998 153(3):767-83]. The final GFAP-IL-1β^{XAT} construct is illustrated in FIG. 16. These constructs were tested by stable transfection into the rat astrocyte line RBA2 [Lee Y C, et al., Brain Res Mol Brain Res. 2003 111(1-2):61-73] using G418 selection. Isolated stable cell lines were analyzed for evidence of successful recombination following transient transfection with pRC-CMV-Cre or viral infection with FIV-Cre by PCR amplification. FIG. 17 illustrates representative results for RAP^{XAT}. Recombination of RAP^{XAT} was shown by PCR amplification of DNA extracts with primers GFAP-RECTEST (binds to the 3′ end of the GFAP promoter) and HSIL-1RA-LP (FIG. 17a). Expression of specific human cytokines (IL-1β or IL-1RA) following recombination was confirmed by ELISA (R & D Systems; FIG. 17b). Further evidence for successful recombination was obtained by X-gal histochemistry, which showed expression of the IRES-lacZ gene only in cells transfected with pRC-CMV-Cre or infected with FIV-Cre (FIG. 17c).

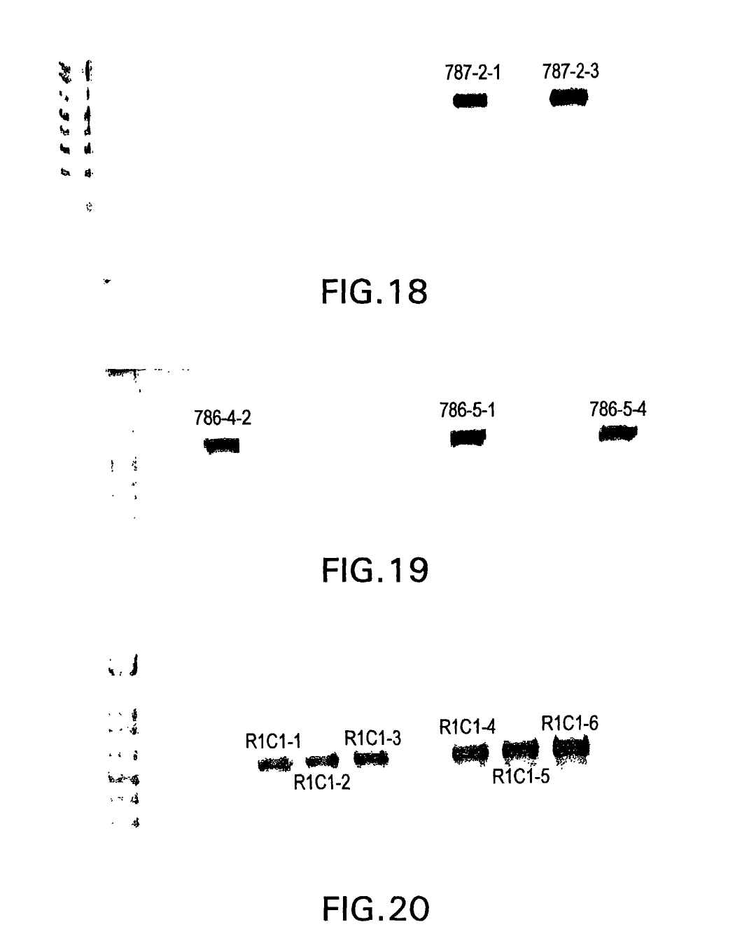

The IL-1 transgene constructs were linearized, purified and injected into fertilized mouse eggs, then reimplanted into pseudopregnant mothers by the University of Rochester Transgenic core facility. Genomic DNA obtained from tail snips of founder mice enabled transgene screening by standard and real-time quantitative PCR (QRT-PCR). Of 11 live IL-1β^{XAT} founders, 2 carried their transgene (FIG. 18). Of 30 live RAP^{XAT} founders screened, 3 carried the transgene (FIG. 19). Initial analysis of transgenic founders indicated that transgenes were present at gene copy numbers of 5 to 20 per cell. The IL-1β^{XAT} transgene was successfully passed from each of the 2 founders to the F1 generation lines L1A and L1B, while to date one RAP^{XAT} founder has passed the transgene to the F1 generation of line R1C (FIG. 20). These lines can be bred to produce heterozygote and homozygote F2 transgenic lines.

c) Creation of Coll1-IL-1β^{XAT}, Coll1-RAP^{XAT}, and Mice Harboring these Transgenes

The rat Col1a1 promoter was kindly donated to us by Dr. Barbara Kream (University of Connecticut) in the pUC12 plasmid without an MTA. The 3.6 Kb promoter sequence was excised following Xba I digestion of the aforementioned plasmid, gel purified and then cloned into the following plasmid containing a custom made cloning site.

A custom made cloning site was prepared by direct DNA-oligo synthesis through the commercially available Gibco/BRL service employing the following sequences:

322. Upper strand:

(SEQ ID NO:75)

^{5′}ATT AAT TAA TCG ATG CGG CCG CTC TAG ATT AAT TAA
TA^{3′}
323. Lower strand:

(SEQ ID NO:76)

^{5′}TAA TTA ATT AGC TAC GCC GGC GAG ATC TAT TTA ATT
AT^{3′}

The two oligos were then hybridized via a single PCR cycle using Taq polymerase, and subsequently cloned directly into the pCRII-Topo vector (Invitrogen, Carlsbad Calif.) per manufacturer's instructions. The pCRII-Topo vector's Xba I site was excised by EcoR I-Apa I digestion, DNA blunting and re-ligation using standard molecular biology methods.

The XbaI-linearized Col1a1 promoter was cloned into the XBA I site of custom-made cloning vector and 5′- to −3′ orientation was confirmed. Next, the Col1a1 promoter containing Pac I-Pac I sequence was excised by restriction enzyme digestion (Pac I), gel purified and cloned into the Pac I site of pBigT/CMV equals IL-1^{XAT} vector (described herein in Example 4a) after simultaneous excision of the present CMV sequences, generating the desired COL1-IL1β^{XAT} transgene (pCOL1-IL1β^{XAT} vector).

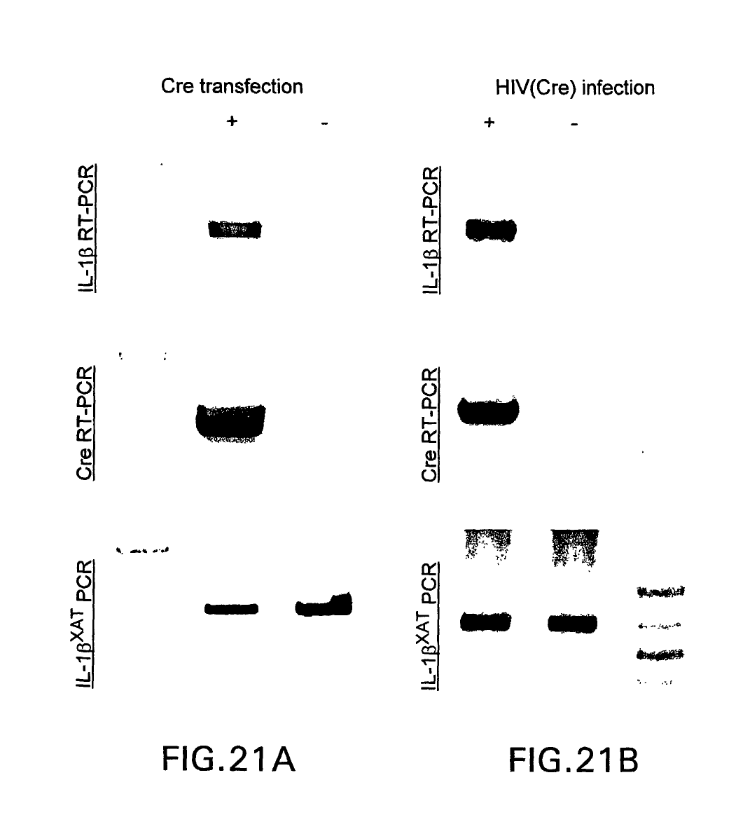

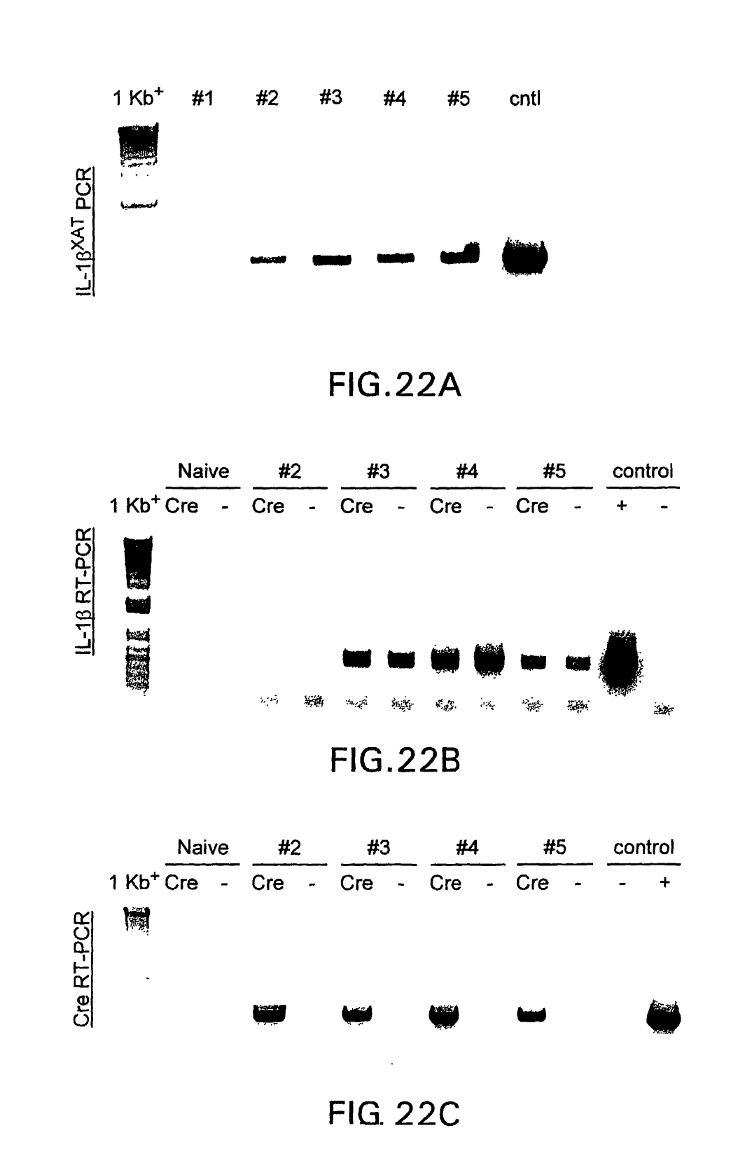

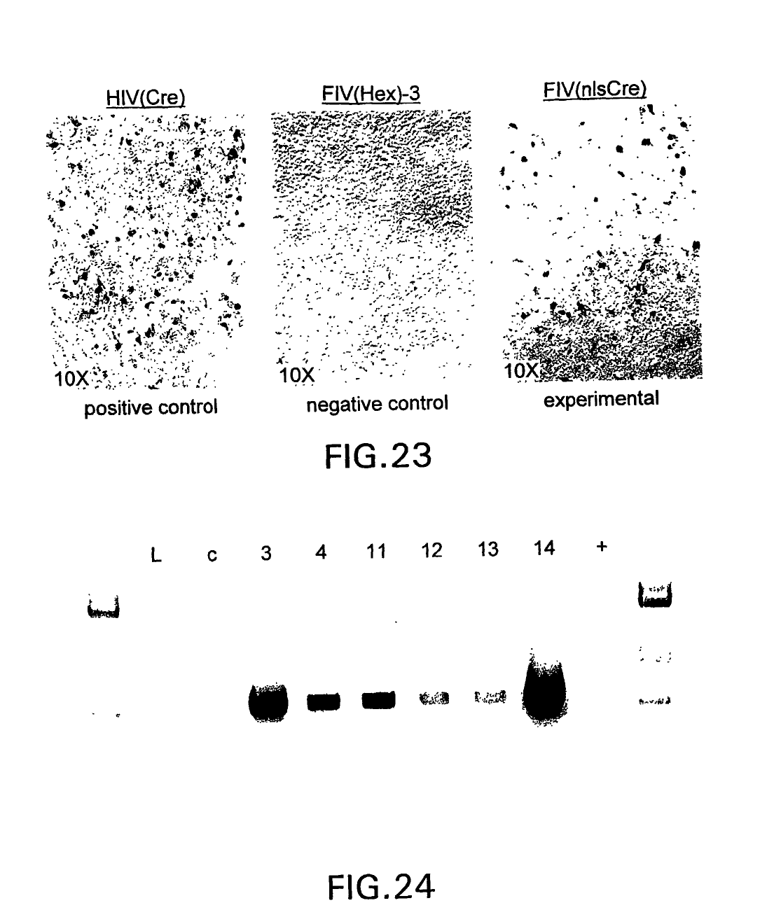

The regulation of COL1-IL1β^{XAT} by Cre recombinase was tested in the murine NIH 3T3 fibroblast cell line as follows. The COL1-IL1β^{XAT} vector was transiently co-transfected together with the HIV(cre) vector into NIH 3T3 cells employing the Lipofectamine 2000 reagent (Invitrogen) per manufacturer's instructions. Please refer to FIG. 21A. In brief, Cre successfully induced the expression COL1-IL1β^{XAT} in vitro as assessed by the expression of IL-1β mRNA. Furthermore, the HIV(Cre) vector was packaged in the 293FT packaging cell line with the aid of vectors pLP1, pLP2 and pLP/VSVG vectors (Invitrogen) per manufacturer's instructions. The virus was then used to infect a stable cell line inherent of the COL1-IL1β^{XAT} gene (FIG. 21B) (FIG. 22). In brief, infection of NIH 3T3 cells that were previously transfected with the COL1-IL1β^{XAT} vector resulted in the expression of IL-1β mRNA.

The HIV(Cre) vector was developed as follows. The commercially available pLenti6/V5-D-Topo system (Invitrogen) was employed. The fusion gene containing the nuclear localization sequence (nls) and the bacterial cre recombinase gene was developed off the pCrePr^{H} vector (Kyrkanides et al. Transcriptional and post-translational regulation of Cre recombinase by RU486 as the basis for an enhanced inducible expression system. Molecular Therapy 8: 790-795, 2004; see also PCT/US03/13672 which is herein incorporated by reference at least for material related to vector production) by PCR using the following primers:

(SEQ ID NO:71)

| | |
|---|---|
| 328. Upper primer: | TCC AAT TTA CTG ACC GTA CAC C |

(SEQ ID NO:72)

| | |
|---|---|
| 329. Lower primer: | GCA ACA CCA TTT TTT CTG ACC |

The subsequent PCR product was directly cloned into the pLenti6/V5-D-Topo vector per manufacturer's instructions.

The COL1-IL1β^{XAT} stable cell lines were developed by transfecting NIH 3T3 cells with the Not 1-Not I segment of the pCOL1-IL1β^{XAT} vector using the Lipofectamine 2000 reagent (Invitrogen) per manufacturer's instructions and subsequently challenging the cells with the antibiotic G418 (1,000 mg/mL). Surviving clones were then picked, expanded and analyzed by PCR for the presence of the COL1-IL1β^{XAT} gene by PCR (FIG. 22A). The cell clones were expanded and further maintained under 400 mg/mL of G418. The inducibility of the COL1-IL1β^{XAT} gene was studied by RT-PCR for the human IL-1β (FIG. 22B) and the expression of Cre recombinase in these cells was confirmed by RT-PCR for Cre recombinase (FIG. 22C).

A new Cre viral vector was developed on the feline immunodeficiency virus system from SBI (Mountain View, Calif.). In brief, the lacZ gene was excised from the vector and the backbone was gel purified. FIV(nls) Cre was constructed with SBI FIVLacZ backbone (excised the LacZ sequence from XbaI to SalI sites) and add the insert of nlsCre sequence from HIV(Cre) by Spe1 and Bpu11021 enzyme digestions. The backbone and insert DNAs were blunted at both ends before the ligation. Subsequently, NIH 3T3 cells were infected with the FIV(nlsCre) virus and subsequently transfected with the CMV-IL1β^{XAT} gene.

TABLE 9
COL1-IL-1β^{XAT} transgenic mouse: locomotive dysfunction
after FIV(nlsCre) injection
Originating from mouse #13 (born on May 18, 2004 - injected
on Sep. 15, 2004)

| | #36 −/− | #37 +/− | #38 +/− | #39 −/− |
|---|---|---|---|---|
| F2 Female | FIV-nlsCre | FIV-nlsCre | FIV-Hex | Saline |
| Oct. 19, 2004 | 21.04 gm | 17.7 gm | 20.89 gm | 21.18 gm |
| | 1′ 48″ | 0′ 4″ | 3′ 59″ | 9′ 26″ |
| | 1′ 48″ | 0′ 2″ | died | 0′ 2″ |
| Oct. 26, 2004 | 22.62 gm | 18.36 gm | — | 21.01 gm |
| | 1′ 22″ | 0′ 19″ | — | 3′ 55″ |
| | 1′ 35″ | 0′ 44″ | — | 0′ 11″ |
| Nov. 2, 2004 | 22.7 gm | 18.36 gm | | 21.01 gm |
| | 1′ 08″ | 0′ 3″ | — | 1′ 08″ |
| | 1′ 10″ | 0′ 15″ | — | 0′ 47″ |
| | | 0′ 15″ | | |

Originating from mouse #14 (born on May 18, 2004 - injected
on Sep. 16, 2004)

| | #45 −/− | #46 +/− | #48 +/− | #47 −/− |
|---|---|---|---|---|
| F2 Female | FIV-nlsCre | FIV-nlsCre | FIV-Hex | Saline |
| Oct. 19, 2004 | 20.02 gm | 19.76 gm | 20.18 gm | 18.29 gm |
| | 10′ 00″ | 3′ 05″ | 0′ 29″ | 10′ 00″ |
| | | | 3′ 15″ | |
| Oct. 26, 2004 | 20.90 gm | 21.51 gm | 20.82 gm | 18.89 gm |
| | 3′ 45″ | 2′ 15″ | 0′ 33″ | 1′ 37″ |
| | | | 3′ 45″ | 2′ 22″ |
| Nov. 2, 2004 | 21.18 gm | 21.22 gm | 20.8 gm | 20.24 gm |
| | 1′ 41″ | 1′ 21″ | 0′ 11″ | 1′ 03″ |
| | 0′ 51″ | 3′ 48″ | 0′ 04″ | 4′ 54″ |
| | | | 0′ 10″ | |

Two mouse sublines (#13 and #14) originating from founder #4 have been bilaterally injected with FIV(cre) into the knee joint and are being monitored weekly for changes in locomotive behavior by the rotorod appliance (at 20 rpm) and mass (in grams).

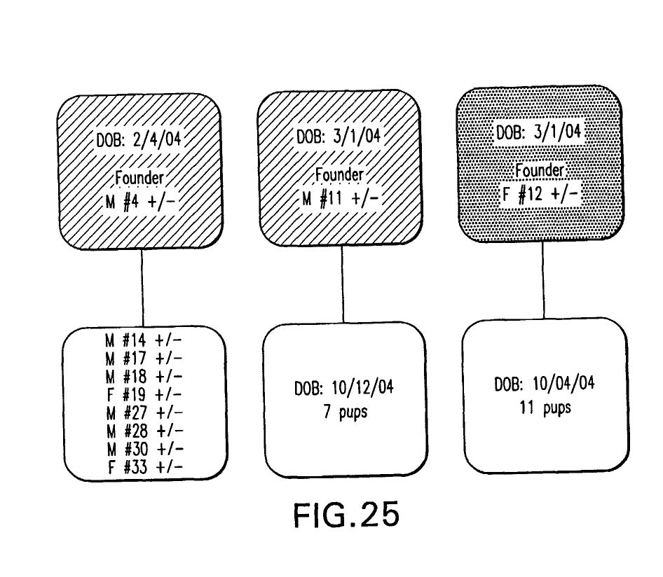

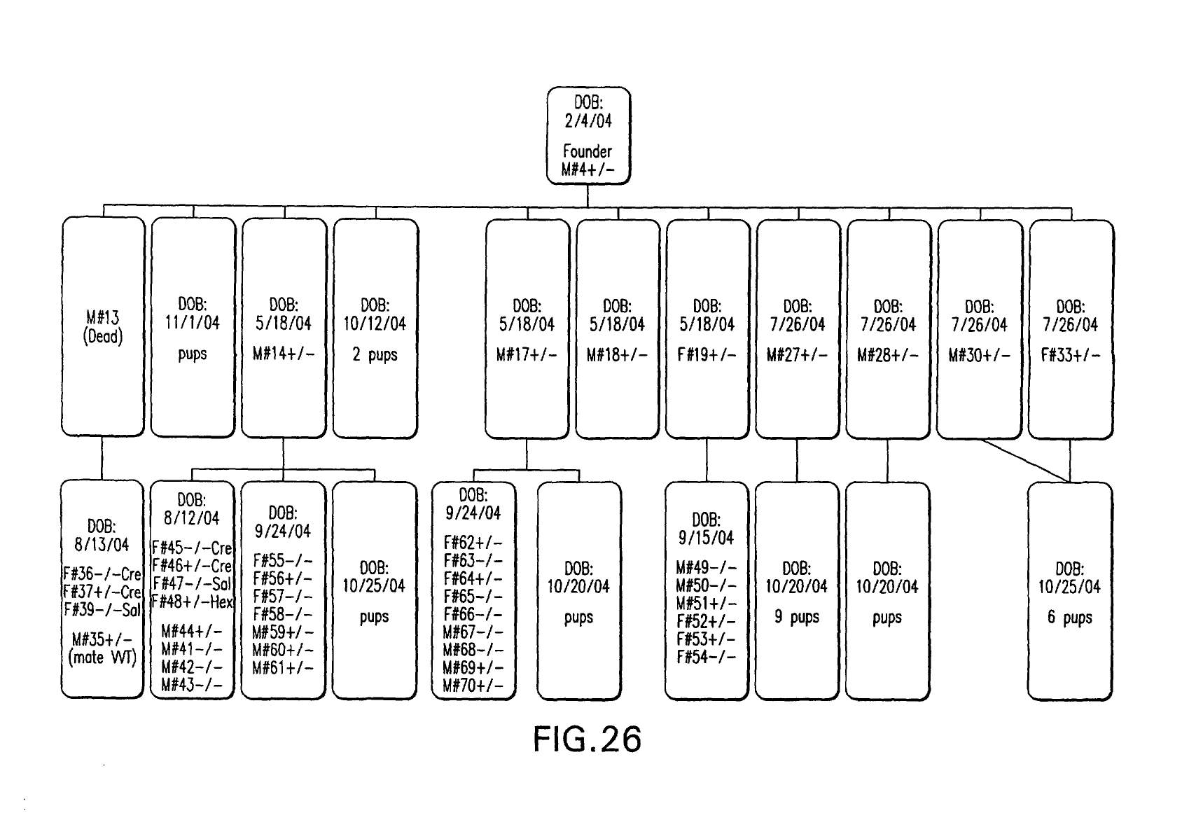

COL1-IL1β^{XAT} transgenic mice were generated in the UofR Transgenic Facility. Not I-Not_linearized fragment from the pCOL1-IL1β^{XAT} was gel purified and prepared following the facilities protocol. The fragment was microinjected in fertilized C57BL/6 oocytes and subsequently implanted into pseudo-pregnant mothers. Thus far, the strategy has yielded 8 pups, of which 3 were identified as positive founders by PCR of genomic DNA extracted from tail snips employing primers specifically designed against the IL1β^{XAT} transgene (FIG. 24). The 3 founders have been bred with C57B1/6 wild type stock mice for analysis of germ-line transmission. Details on the offspring have been provided in FIG. 25 and FIG. 26.

Administration of FIV(nlsCre) in vivo in both temporomandibular joints and knee joints was performed via intra-articular injection of 100 μl viral solution. The mice have been followed behaviorally and have found that the FIV(nlsCre) mice developed phenotypic changes that are expected in situations of inflammatory joint disease: decreased locomotion. Please see Table 9 for details.

d) Transgene Activation in Joints of Col1-IL1β^{XAT} Mice

In order to evaluate the effect of transgene activation in the joints of Col1-IL1β^{XAT} mice, two sets of Col1A1-IL1β^{XAT} mice received intra-articular FIV(Cre) injections (a total of 10⁶ infectious particles) in the right and left knee, as well as the left and right temporomandibular joint (TMJ). The mice were monitored over a period of 8 weeks for changes in grooming behavior and locomotion. The mice were subsequently sacrificed and their knees and TMJs were histologically analyzed.

Behavioral changes were assessed as previously described (Dubuisson, D. and Dennis, S. Pain 1977; 4: 161-74; Abbott F V, et al. Eur J Pharmacol 1986; 126:126-41), which are herein incorporated by reference for teachings related to these methods. In brief, a group of Col1-IL1β^{XAT} transgenic mice (N=3) received a single intra-articular injection of 10⁶ infectious particles of FIV(Cre) in the right and left knees at 2 months of age. In addition, a second group of mice (N=3) received saline injection and served as controls. During a session, each mouse was videotaped for 1 hour. The tape was then transferred digitally to a computer and analyzed in 20 periods of 3 minutes each. The duration of each mouse displaying grooming and licking was recorded and summed as seconds by an investigator who was blind to the animal group assignment. Injection of FIV(Cre) into the knee of Col1-IL1β^{XAT} transgenic mice resulted in a four-fold increase in the duration of grooming as compared to saline-injected controls (FIG. 9, P<0.05).

Four groups of mice (N-3) were evaluated in terms of locomotive behavior by the rotorod appliance (Columbus Instruments, Columbus Ohio) and the lapse time until the mice fell off the rotating cylinder (20 rpm) was recorded. The mice were evaluated over a period of 8 weeks following the intra-articular injections (8 wks-16 wks of age). As seen in FIG. 10, it was demonstrated that FIV(Cre)-injected Col1-IL1β^{XAT} transgenic mice developed significant locomotive deterioration (Tg+Cre) compared to transgenic mice injected with the control FIV(gfp) vector (TG+gfp), as well as the other control animals groups (WT-Cre & WT-saline).

Immunocytochemical detection of the reporter gene β-galactosidase was employed to confirm the activation of the Col1-IL1β^{XAT} transgene by FIV(Cre) in this mouse model using antibodies raised against β-galactosidase and Cre recombinase. Shown in FIG. 11 is FITC-conjugated immunodetection of β-galactosidase (FIG. 11A), Texas Red-conjugated immunodetection of Cre recombinase (FIG. 11B), B/W image of the same microscopic field (FIG. 11C), overlap of panels A+B (FIG. 11D), and overlap of panels A+B+C (FIG. 11E). Demonstrated is the co-expression of β-galactosidase and Cre recombinase in vivo (FIG. 11, solid arrows). Note that there are more red cells than green cells (FIG. 11, open arrows) indicating that not all infected cells express the transgene Col1A1→IL1β-RES-lacZ in the same capacity.

H&E staining of a knee section harvested from a 4 month old Col1-IL1β^{XAT} transgenic mouse injected with FIV(Cre) revealed the formation of fibrillations (FIG. 12A, solid arrow) and of an articular lip (FIG. 12B, open arrow). In contrast, a transgenic mouse that received the control vector FIV(GFP) did not develop such anatomic aberrations (FIG. 12B). Alcian blue/orange semi-quantitative evaluation showed a decrease in cartilage (FIG. 12C, less blue stain) and bone (FIG. 12D, less red stain) density in the Col1-IL1β^{XAT}+FIV(Cre) knees compared to controls (FIG. 12E). Moreover, increased cloning along with thickening of the articular surfaces was observed in the experimental animals (FIG. 12C, indicated by small arrows). These observations indicate the presence of arthritis in the knee following transgene induction by Cre recombinase.

Eight weeks after FIV(Cre) injection in the knee and TMJ of Col1-IL1β^{XAT} mice, the brain was evaluated for activation of microglia and astrocytes by immunocytochemistry. Using a monoclonal antibody raised against the MHC-class II antigen, the presence of activated microglia was detected in the brain (FIGS. 13A, C). In contrast, control animals did not display any MHC-II positive cells. Interestingly, there was lack of astrocyte activation in the brains of these animals as assessed by glial fibrillary acidic protein (GFAP) (FIGS. 13B, D). In general, control animals (inactive transgenic mice) displayed no signs of brain inflammation by MHC-II or GFAP immunocytochemistry.

Eight weeks after FIV(Cre) injection in the TMJ of Col1-IL1β^{XAT} mice anatomic aberrations of the joint were evaluated by semi-quantitative Alcian blue—orange G histochemistry. Shown in FIGS. 14A and C are a TMJ section from an inactive Col1-IL1β^{XAT} mouse depicting the condylar head as well as the meniscus. In comparison, FIGS. 14B and C depict a TMJ section harvested from a Col1-IL1β^{XAT} mouse injected with FIV(Cre) in the TMJ. An apparent reorganization of the TMJ cell layers was observed following FIV(Cre) injection, whereby a loss of the most superficial cell layer was noted accompanied by disorganization of the proliferative layer of chondrocytes (FIG. 14, open arrows). In addition, a decrease in cartilage content was observed in the condylar head of FIV(Cre)-treated Col1-IL1β^{XAT} mice as evaluated semi-quantitatively by Alcian blue—orange G histochemistry (FIG. 14, purple/blue stain).

### Example 5

### Transgene Activation in Joints of Col1-IL1β^{XAT} Mice

In order to evaluate the effect of transgene activation in the joints of Col1-IL1β^{XAT} mice, two sets of Col1A1-IL1β^{XAT} mice received intra-articular FW(Cre) injections (a total of 10⁶ infectious particles) in the right and left knee, as well as the left and right temporomandibular joint (TMJ). The mice were monitored over a period of 8 weeks for changes in grooming behavior and locomotion. The mice were subsequently sacrificed and their knees and TMJs were histologically analyzed.

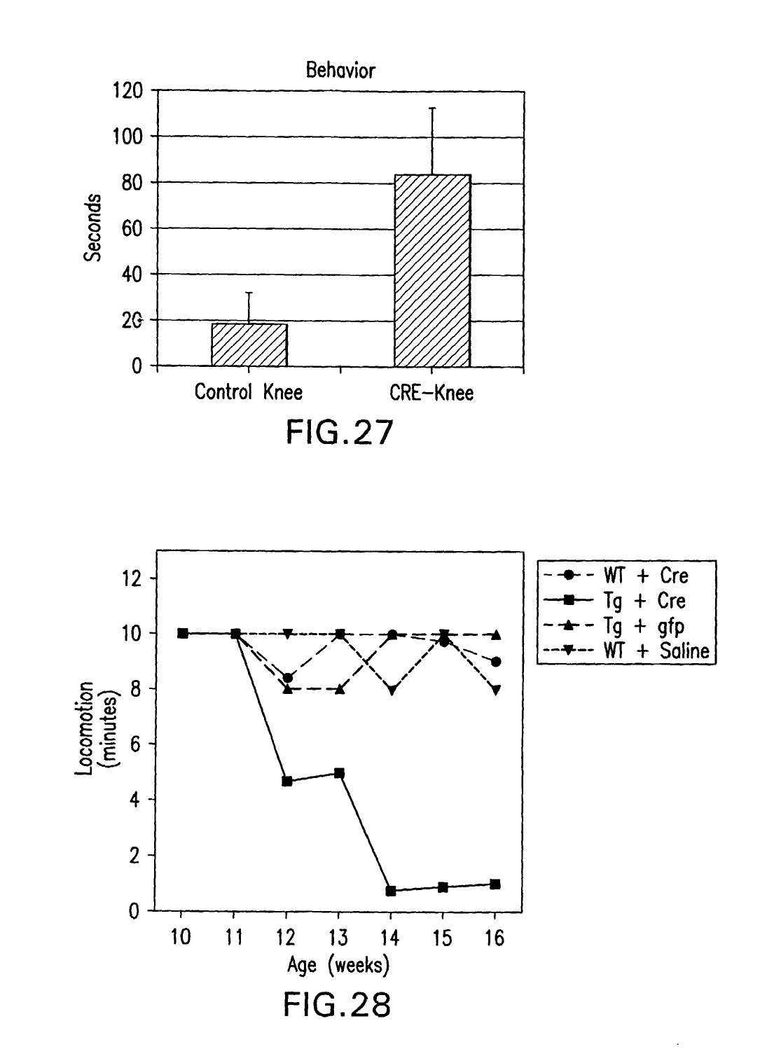

Behavioral changes were assessed as previously described (Dubuisson, D. and Dennis, S. Pain 1977; 4: 161-74; Abbott F V, et al. Eur J Pharmacol 1986; 126:126-41), which are herein incorporated by reference for teachings related to these methods. In brief, a group of Col1-IL1β^{XAT} transgenic mice (N=3) received a single intra-articular injection of 10⁶ infectious particles of FIV(Cre) in the right and left knees at 2 months of age. In addition, a second group of mice (N=3) received saline injection and served as controls. During a session, each mouse was videotaped for 1 hour. The tape was then transferred digitally to a computer and analyzed in 20 periods of 3 minutes each. The duration of each mouse displaying grooming and licking was recorded and summed as seconds by an investigator who was blind to the animal group assignment. Injection of FIV(Cre) into the knee of Col1-IL1β^{XAT} transgenic mice resulted in a four-fold increase in the duration of grooming as compared to saline-injected controls (FIG. 27, P<0.05).

Four groups of mice (N=3) were evaluated in terms of locomotive behavior by the rotorod appliance (Columbus Instruments, Columbus Ohio) and the lapse time until the mice fell off the rotating cylinder (20 rpm) was recorded. The mice were evaluated over a period of 8 weeks following the intra-articular injections (8 wks-16 wks of age). As seen in FIG. 28, it was demonstrated that FIV(Cre)-injected Col1-IL1β^{XAT} transgenic mice developed significant locomotive deterioration (Tg+Cre) compared to transgenic mice injected with the control FIV(gfp) vector (TG+gfp), as well as the other control animals groups (WT-Cre & WT-saline).

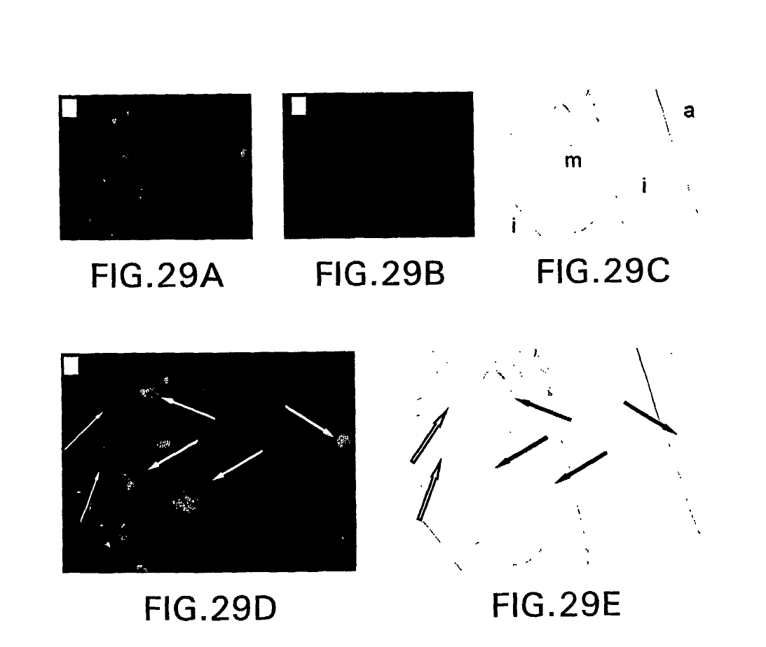

Immunocytochemical detection of the reporter gene β-galactosidase was employed to confirm the activation of the Col1-IL1β^{XAT} transgene by FW(Cre) in this mouse model using antibodies raised against β-galactosidase and Cre recombinase. Shown in FIG. 29 is FITC-conjugated immunodetection of β-galactosidase (FIG. 29A), Texas Red-conjugated immunodetection of Cre recombinase (FIG. 29B), B/W image of the same microscopic field (FIG. 29C), overlap of panels A+B (FIG. 11D), and overlap of panels A+B+C (FIG. 29E). Demonstrated is the co-expression of α-galactosidase and Cre recombinase in vivo (FIG. 29, solid arrows). Note that there are more red cells than green cells (FIG. 29, open arrows) indicating that not all infected cells express the transgene Col1A1→IL1β-IRES-lacZ in the same capacity.

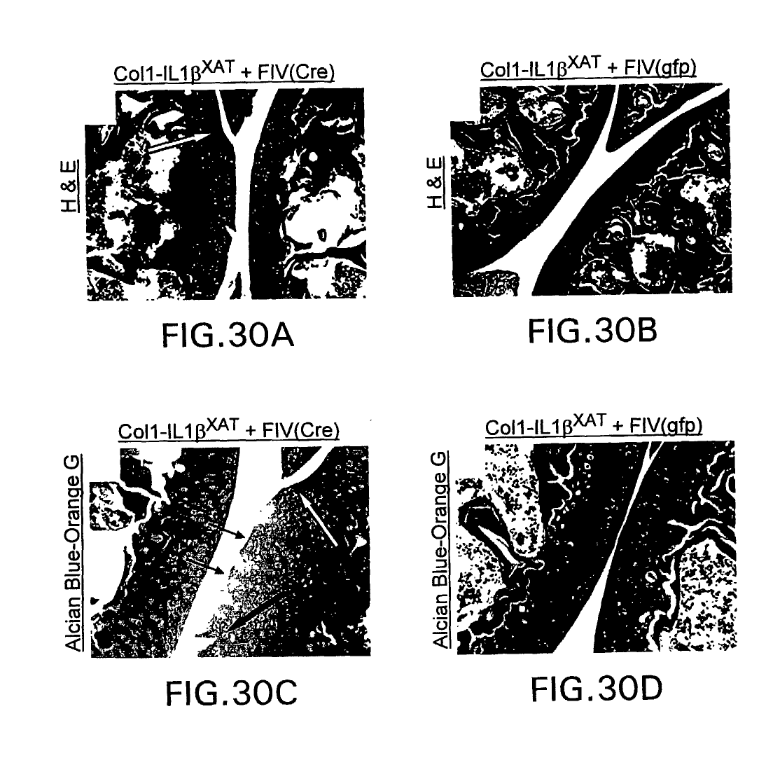

H&E staining of a knee section harvested from a 4 month old Col1-IL1β^{XAT} transgenic mouse injected with FW(Cre) revealed the formation of fibrillations (FIG. 30A, solid arrow) and of an articular lip (FIG. 12B, open arrow). In contrast, a transgenic mouse that received the control vector FIV(GFP) did not develop such anatomic aberrations (FIG. 30B). Alcian blue/orange semi-quantitative evaluation showed a decrease in cartilage (FIG. 30C, less blue stain) and bone (FIG. 30D, less red stain) density in the Col1-IL1β^{XAT}+FIV(Cre) knees compared to controls (FIG. 30E). Moreover, increased cloning along with thickening of the articular surfaces was observed in the experimental animals (FIG. 30C, indicated by small arrows). These observations indicate the presence of arthritis in the knee following transgene induction by Cre recombinase.

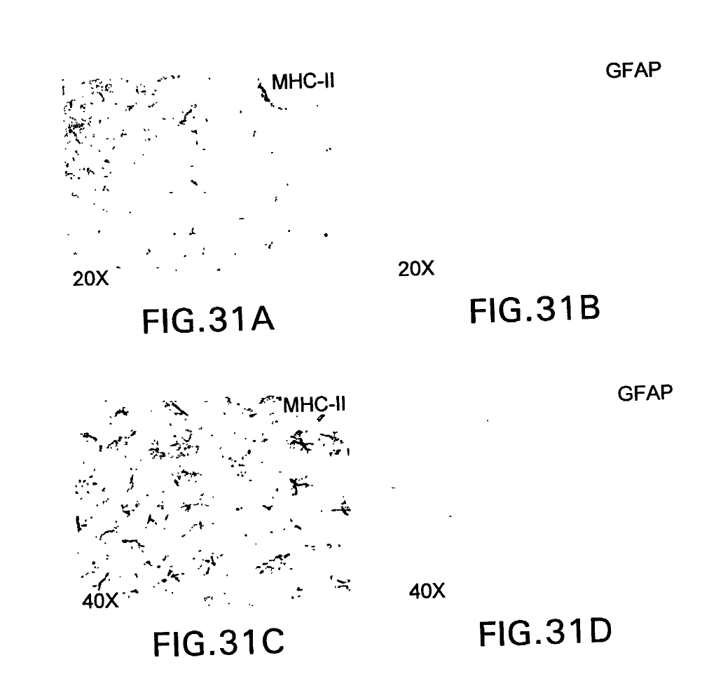

Eight weeks after FIV(Cre) injection in the knee and TMJ of Col1-IL1β^{XAT} mice, the brain was evaluated for activation of microglia and astrocytes by immunocytochemistry. Using a monoclonal antibody raised against the MHC-class II antigen, the presence of activated microglia was detected in the brain (FIGS. 31A, C). In contrast, control animals did not display any MHC-II positive cells. Interestingly, there was lack of astrocyte activation in the brains of these animals as assessed by glial fibrillary acidic protein (GFAP) (FIGS. 31B, D). In general, control animals (inactive transgenic mice) displayed no signs of brain inflammation by MHC-II or GFAP immunocytochemistry.

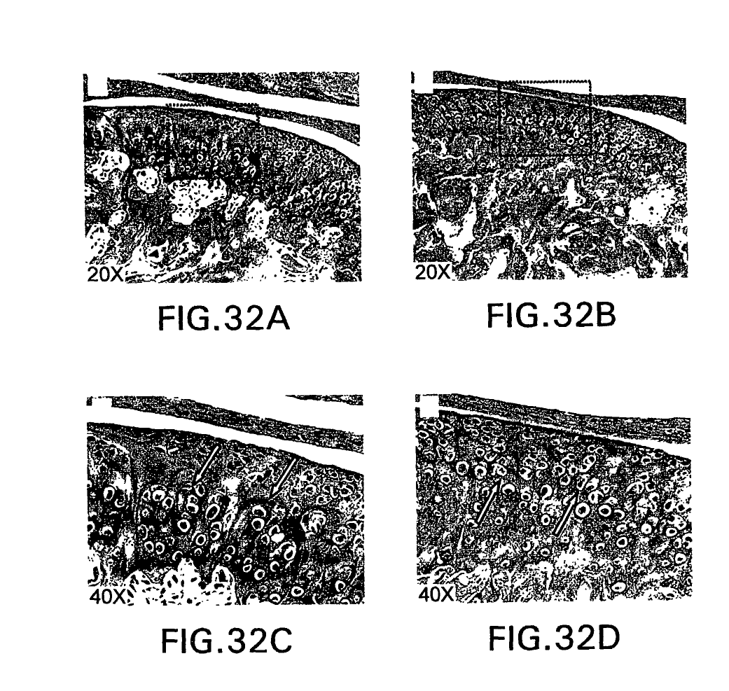

Eight weeks after FIV(Cre) injection in the TMJ of Col1-IL1β^{XAT} mice anatomic aberrations of the joint were evaluated by semi-quantitative Alcian blue—orange G histochemistry. Shown in FIGS. 32A and C are a TMJ section from an inactive Col1-IL1β^{XAT} mouse depicting the condylar head as well as the meniscus. In comparison, FIGS. 32B and C depict a TMJ section harvested from a Col1-IL1β^{XAT} mouse injected with FIV(Cre) in the TMJ. An apparent reorganization of the TMJ cell layers was observed following FIV(Cre) injection, whereby a loss of the most superficial cell layer was noted accompanied by disorganization of the proliferative layer of chondrocytes (FIG. 32, open arrows). In addition, a decrease in cartilage content was observed in the condylar head of FIV(Cre)-treated Col1-IL1^{XAT} mice as evaluated semi-quantitatively by Alcian blue—orange G histochemistry (FIG. 32, purple/blue stain).

### Example 6

### Temporally and Spatially Controlled IL-1β Production in the Adult Mouse Brain

The IL1b-XAT construct was linearized and used to generate 2 transgenic mouse lines (A and B) by the University of Rochester Transgenic Core Facility. Feline immunodeficiency (FIV) based vectors (1.5 ml, ˜1.5 e4 infectious units) were used to deliver Cre (FIV-Cre), green fluorescent protein (FIV-GFP) or LacZ (FIV-LacZ) to the mouse hippocampus under isoflurane anesthesia. Injections were performed at 8-12 weeks of age in heterozygous animals which were provided with food ad libitum. Hippocampal RNA isolation was performed using Trizol, and cDNA was generated using Superscript m (Invitrogen). QRT-PCR was performed using the iCycler (Bio-Rad), and gene transcript levels were determined relative to the housekeeping gene 18s. Immunocytochemistry (ICC) was performed following 4% paraformaldehyde fixation and sucrose immersion. Brains were sliced into 30 mm free floating sections and antibody binding was visualized using diaminobenzidine or Texas Red conjugated antibodies (Vector Labs and Molecular Probes). Gene transcript analysis was performed using student t-tests and 1-way ANOVA with Bonferonni post tests comparing ipsilateral hemispheres from lines A and B with those of control animals (n=3-5 per group).

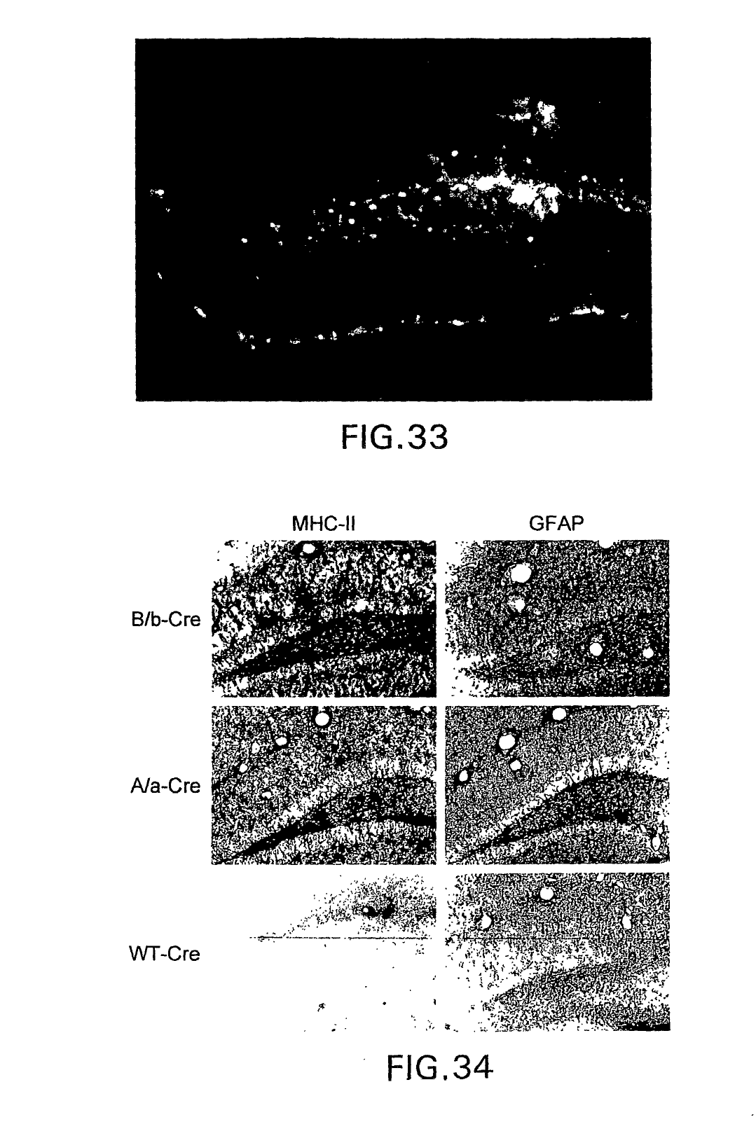

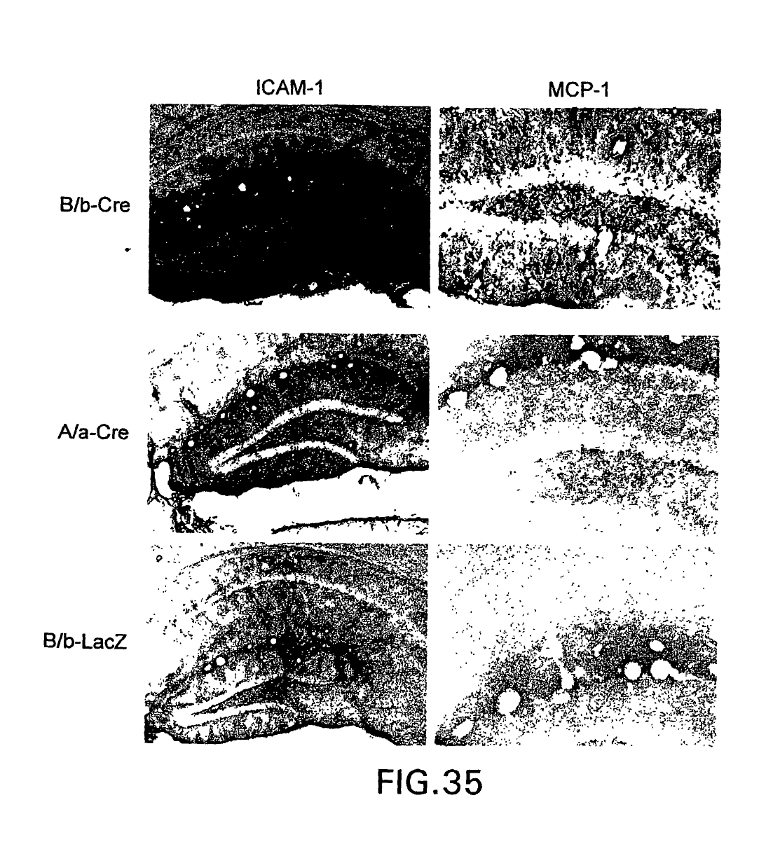

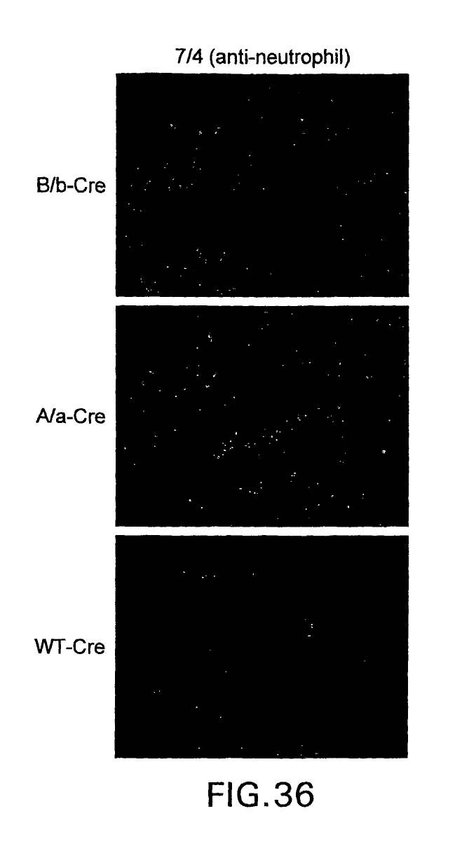

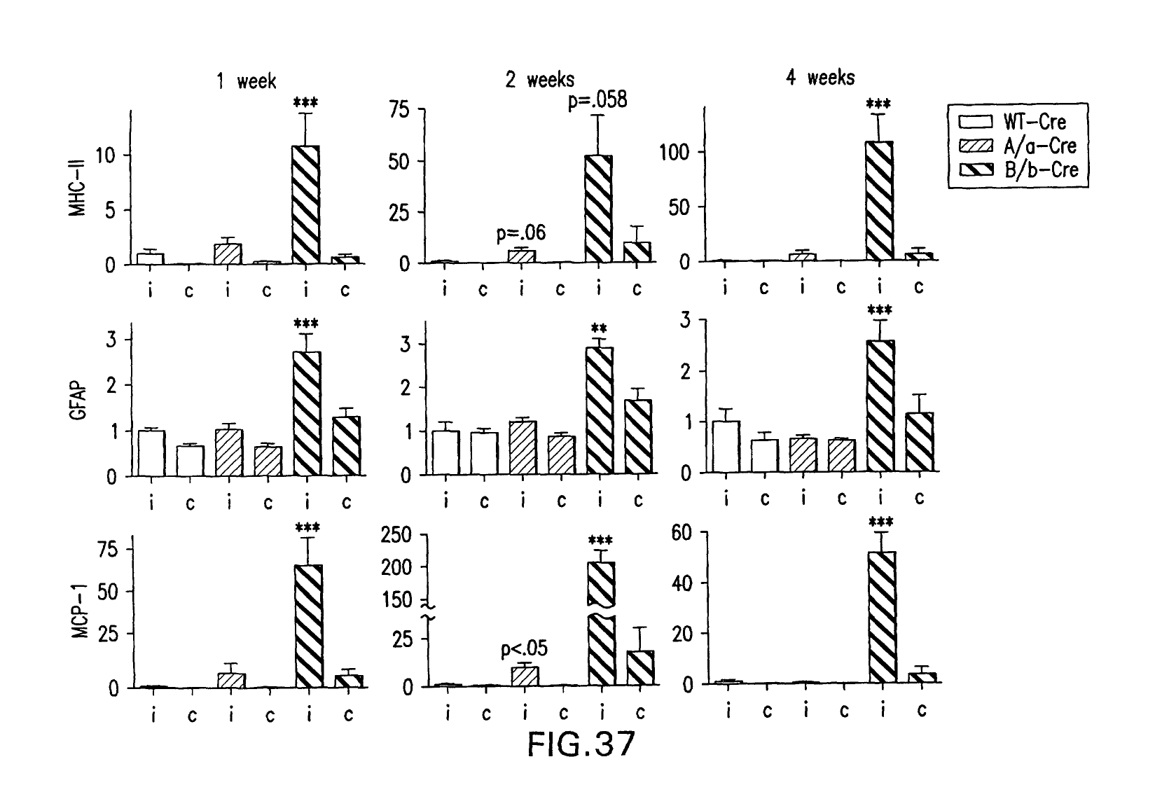

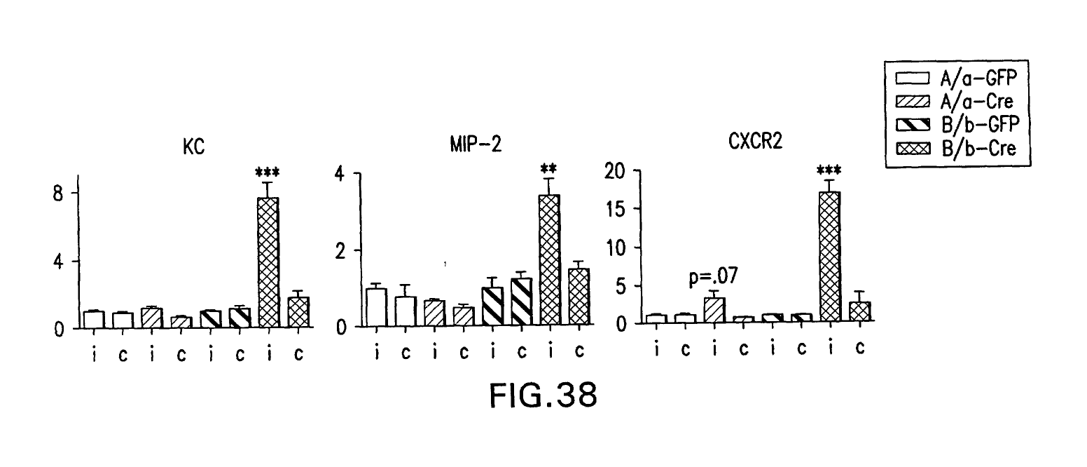

FIG. 33 shows GFP expression in the mouse hippocampus 1 week following FIV-GFP injection. Hippocampal human IL-1β expression leads to a robust neuroinflammatory response consisting of glial activation (FIG. 34) and induction of cytokines (FIG. 35) and chemokines (FIG. 38). IL-1, is a potent driving force for neutrophil recruitment to the hippocampus, and likely involves induction of the ELR+CXC chemokines. Two-weeks following FIV-Cre injection there were numerous neutrophils recruited to the hippocampal parenchyma (B/b>>A/a) as evidenced by 7/4 antibody staining (FIG. 36). The two heterozygous IL-1β-XAT transgenic lines (A/a and B/b) have distinct phenotypes following gene induction (FIG. 37).

### Example 7

### Glial Cell Activation in Peripheral Pain

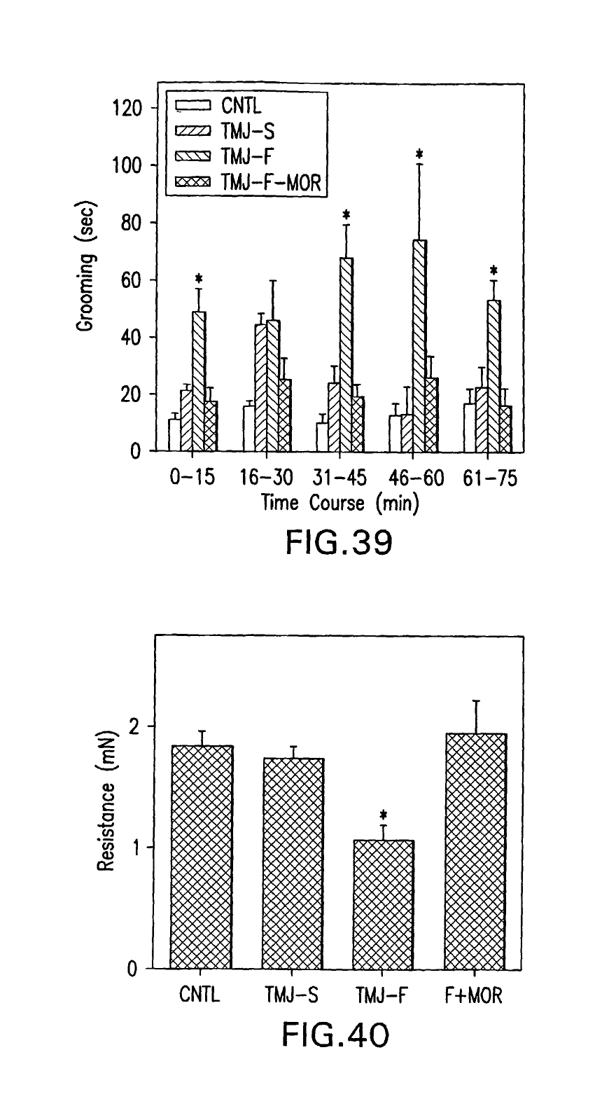

Orofacial grooming is significantly increased in response to the application of painful stimulus (formalin) into the TMJ and normalized following systemic administration of morphine (FIG. 39). Resistance to jaw opening is significantly decreased in response to the application of painful stimulus (formalin) into the TMJ and normalized following systemic administration of morphine (FIG. 40).

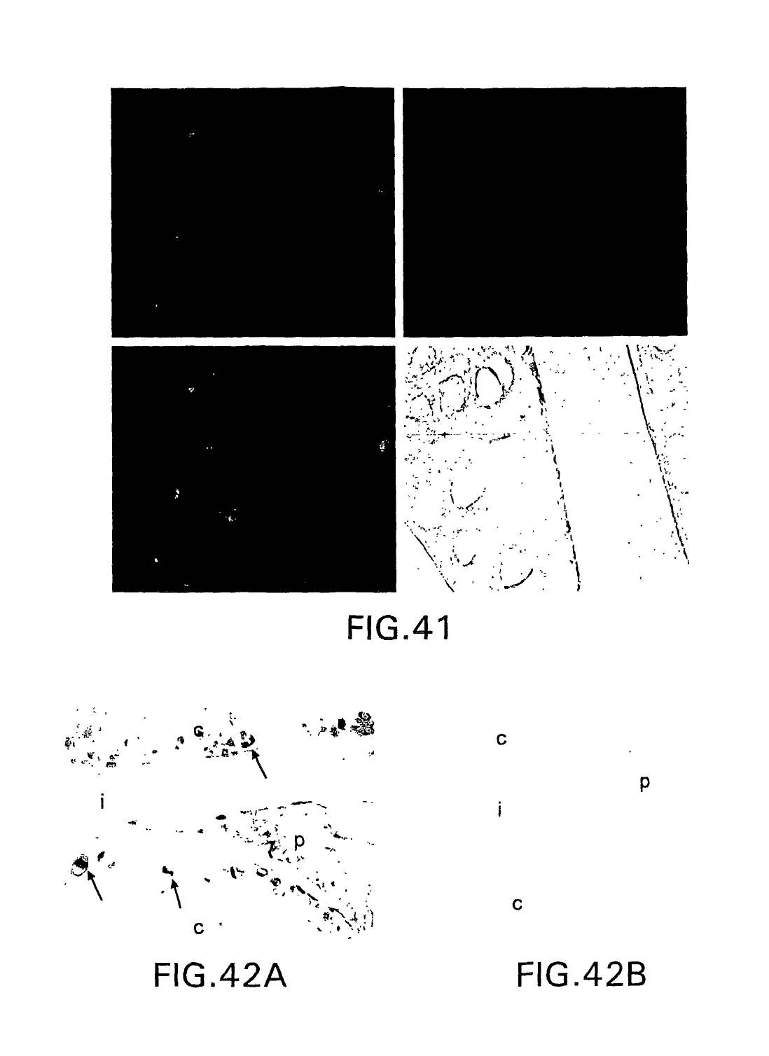

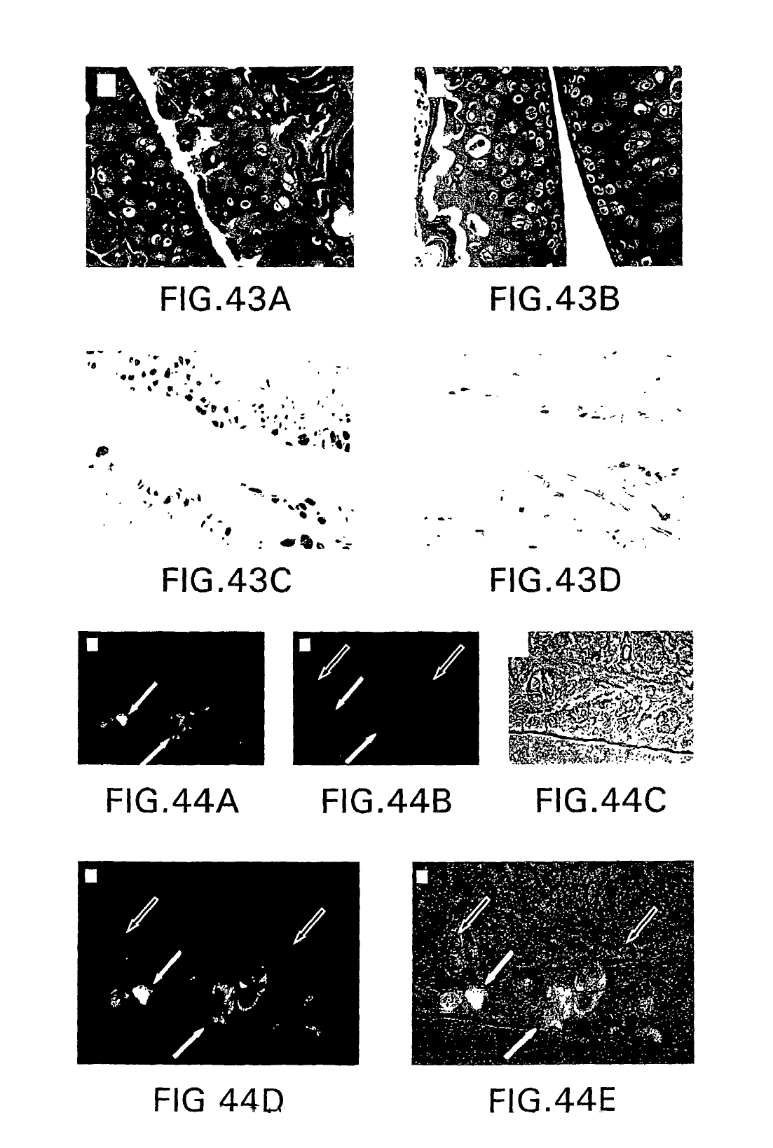

FIV(Cre) injection in the knee of Col1-IL1β^{XAT} mice resulted in transgene induction (FIG. 41) and chronic expression of hIL-1β (FIG. 42), which results in arthritic changes in the knee joint (FIG. 43).

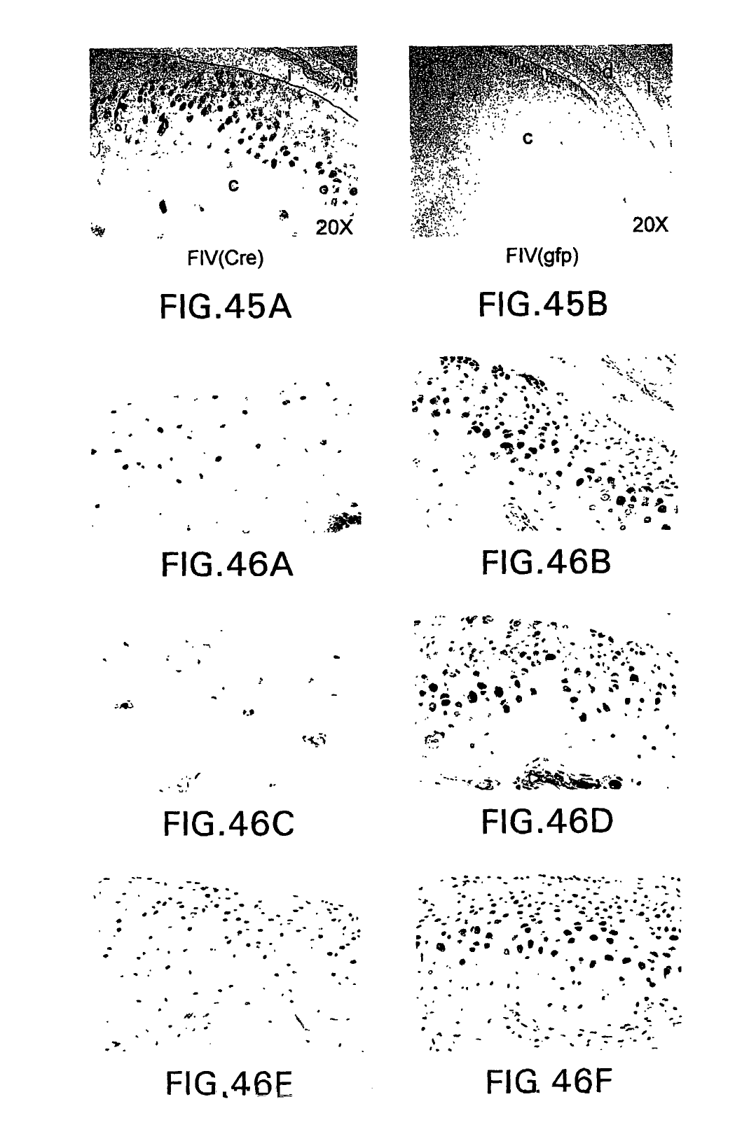

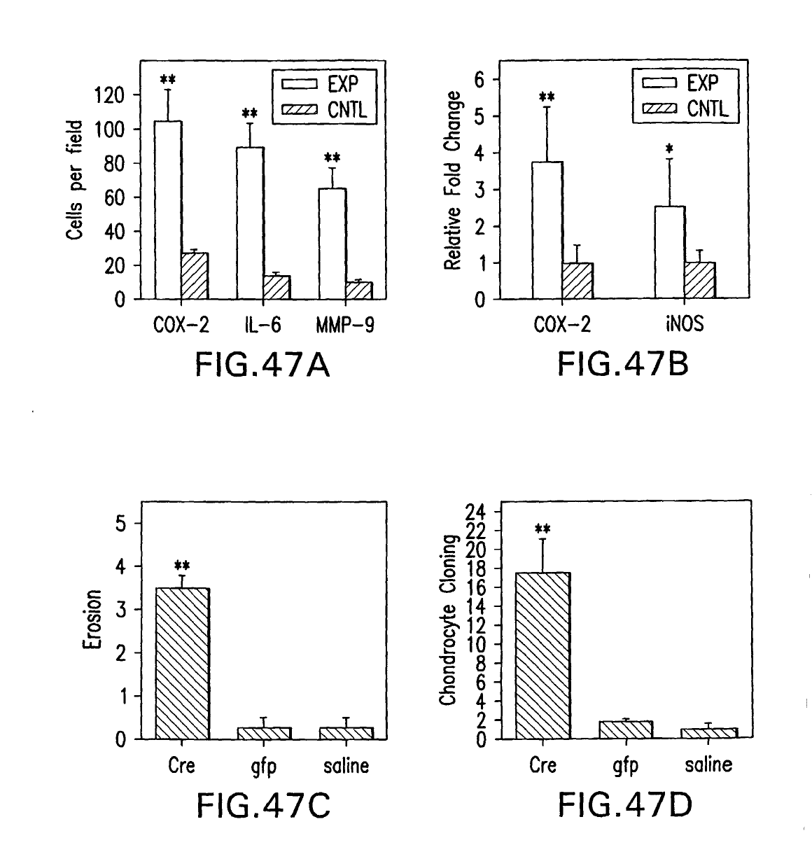

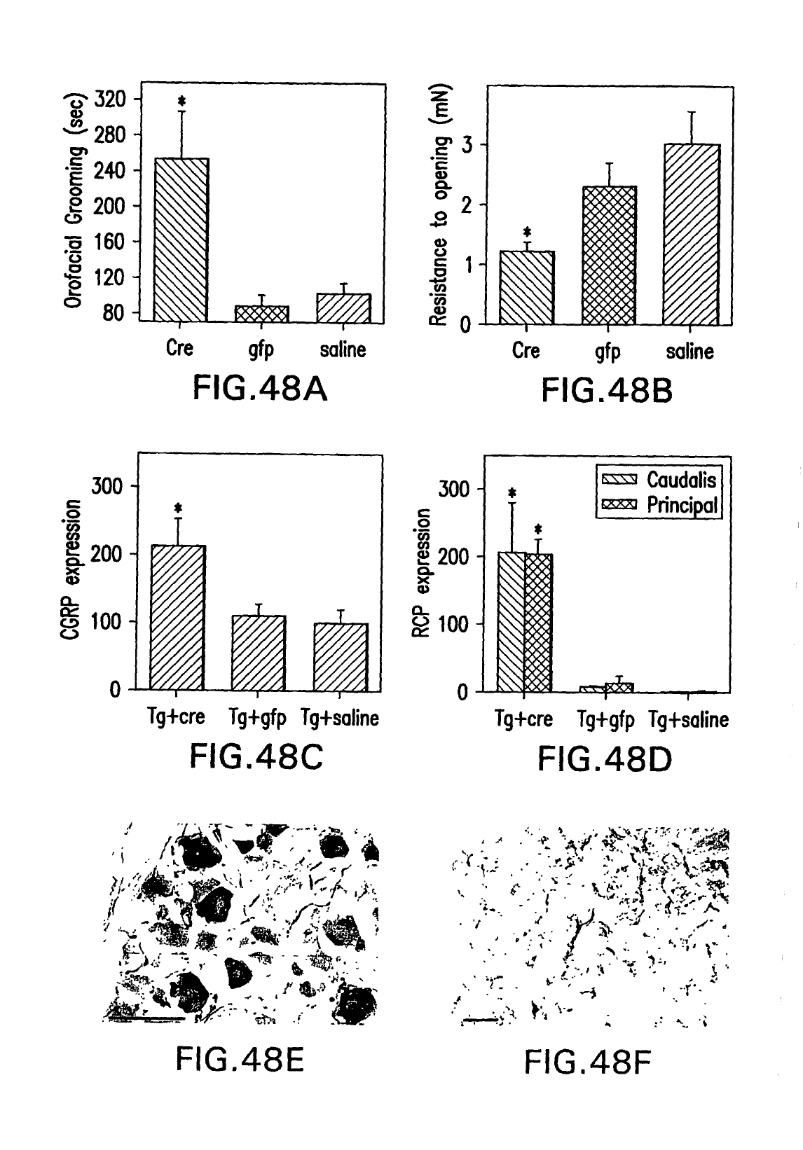

FIV(Cre) injection activates Col1-IL1β^{XAT} gene expression in the TMJ of transgenic mice (FIGS. 44 and 45). As shown in FIG. 46, COL1-IL1βXAT activation in the TMJ induces the expression of inflammatory mediators. Induction of IL-6 in the proliferative zone of the articular surface, as well as (FIGS. 46C-D) increased COX-2 expression. Moreover, MMP-9 (gelatinase B) was also found increased in the experimental mice compared to controls (FIGS. 46E-F) as assessed by immunohistochemistry. Induction of IL-6, COX-2 and MMP-9 indicates the presence of inflammation in the TMJ of adult activated transgenic mice (40×). As further shown in FIG. 47, COL1-IL1βXAT activation in the TMJ induces arthritic changes in the TMJ. Further, Col1-IL1βXAT activation in the adult TMJ results in orofacial pain and joint dysfunction (FIG. 48).

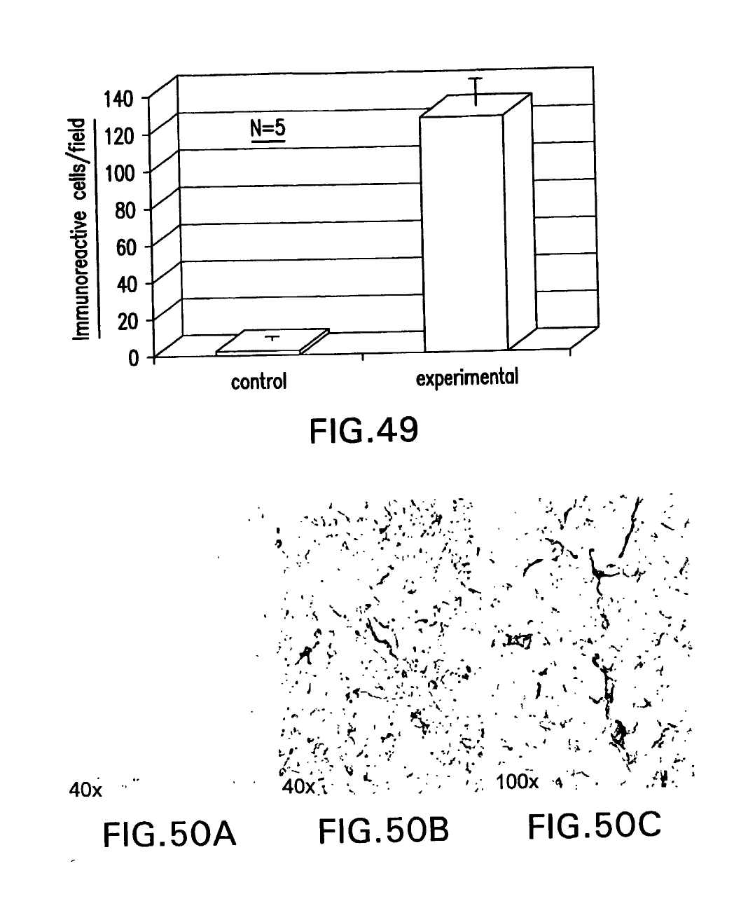

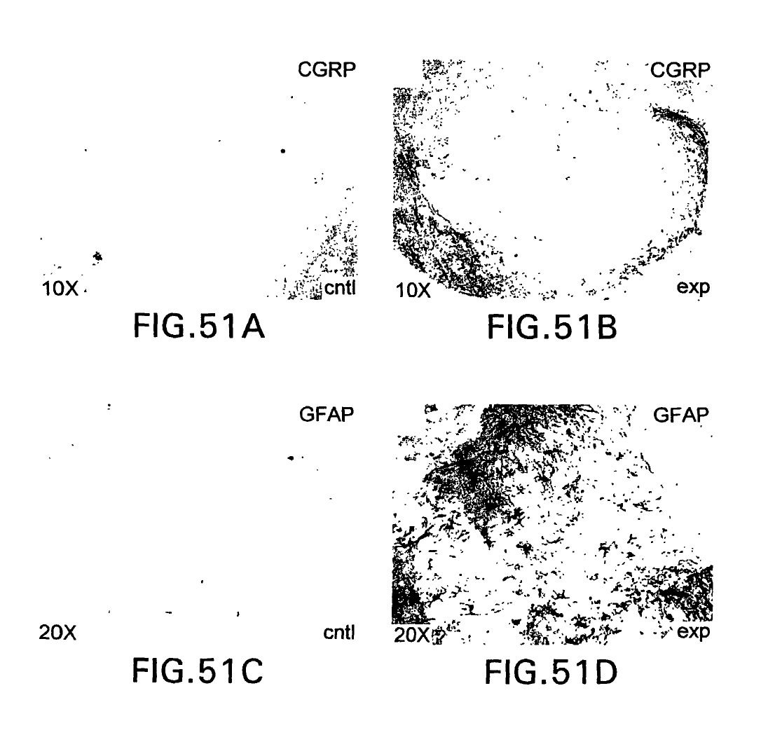

Interestingly, murine IL-1β is induced in the brain stem of mice suffering from chronic TMJ arthritis. Eight weeks following viral transduction, the level of murine IL-1β expression was found significantly increased at the level of the main sensory nuclear of their brain stem compared to FIV(gfp)-injected (control) mice (FIG. 49). Further, there was astrocyte activation (as assessed by GFAP IHC) in the brain stem of Col1-IL1βXAT mice exhibiting TMJ arthritis and pain (FIG. 50). As shown in FIG. 51, IL-1β injection into the cisterna magna induces neuronal excitation and astrocyte activation.

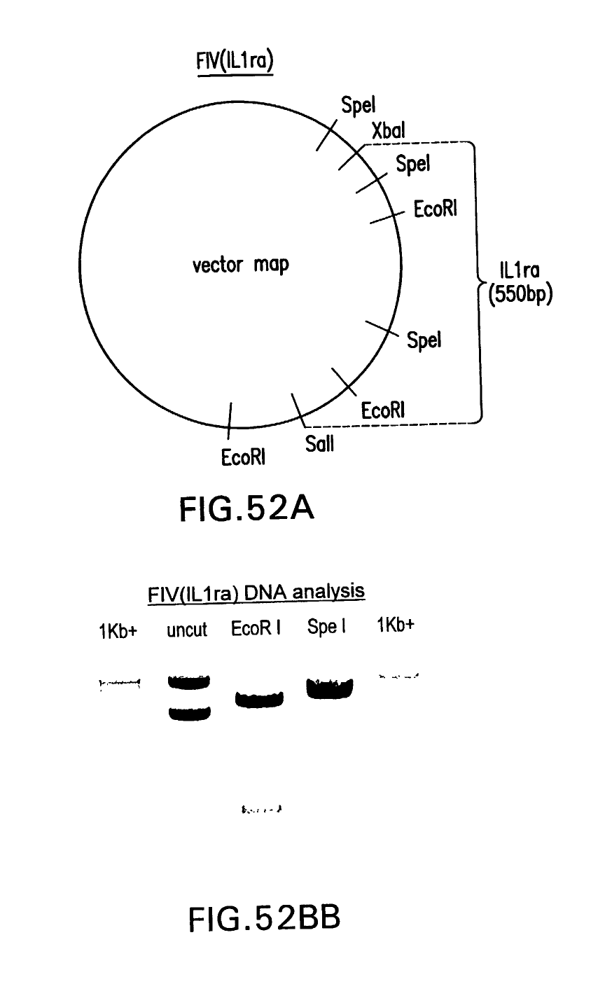

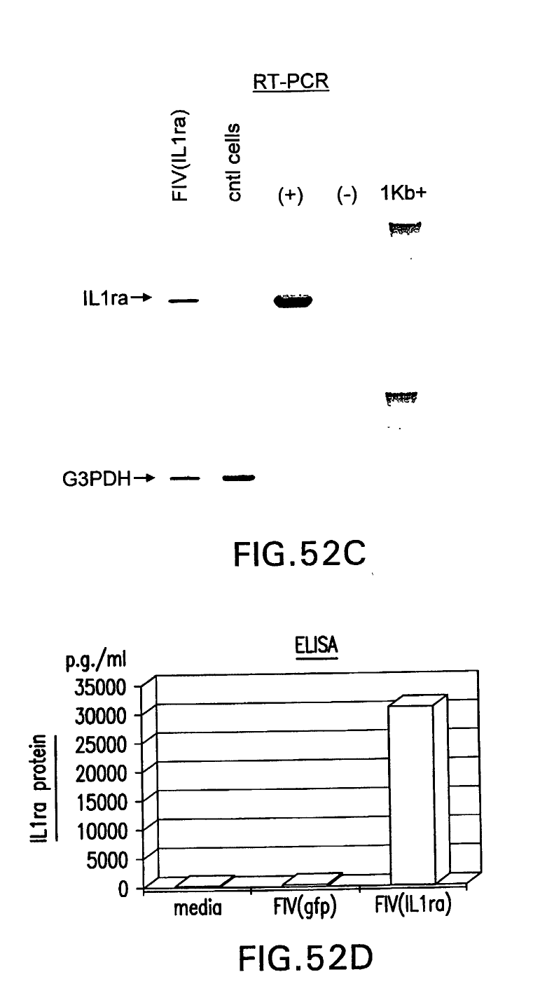

As shown in FIG. 52, FIV(IL1ra) successfully transduces cells with a gene expressing IL-1ra receptor antagonist. Infection of cells by FIV(IL1ra) resulted in therapeutic IL1ra levels (>30 μg/mL).

### F. REFERENCES

Abbott F V, Franklin K B J, Conel B (1986). The stress of a novel environment reduces formalin pain: possible role of serotonin. Eur J Pharmacol 126: 126-41.
Abramowitz M and Stegun I A (1964). Handbook of Mathematical Functions, Applied Mathematics Series, vol. 55 (Washington: National Bureau of Standards; reprinted 1968 by Dover Publications, New York).
Adamo C T, Mailhot J M, Smith A K, Borke J L (2001) Connexin 43 expression in oral derived human osteoblasts after transforming growth factor-beta and PGE2 exposure. J Oral Implantol 27: 25-31
Agarwal S, Long P, Gassner R, Piesco N P, Buckley M J (2001). Cyclic tensile strain suppresses catabolic effects of interleukin-1 beta in fibrochondrocytes from the temporomandibular joint. Arthr Rheum 44: 608-17.
Ahmadzadeh N, Shingu M, Nobunaga M (1990). The effect of recombinant tumor necrosis factor-alpha on superoxide and metalloproteinase production by synovial cell and chondrocytes. Clin Exp Rheumatol 8:387-91.
Akiyama, H., S. Barger, S. Barnum, B. Bradt, J. Bauer, G. M. Cole, N. R. Cooper, P. Eikelenboom, M. Emmerling, B. L. Fiebich, C. E. Finch, S. Frautschy, W. S. Griffin, H. Hampel, M. Hull, G. Landreth, L. Lue, R. Mrak, I. R. Mackenzie, P. L. McGeer, M. K. O'Banion, J. Pachter, G. Pasinetti, C. Plata-Salaman, J. Rogers, R. Rydel, Y. Shen, W. Streit, R. Strohmeyer, I. Tooyoma, F. L. Van Muiswinkel, R. Veerhuis, D. Walker, S. Webster, B. Wegrzyniak, G. Wenk and T. Wyss-Coray. inflammation and Alzheimer's disease. Neurobiol Aging (2000) 21:383-421.
Alisky, J. M., S. M. Hughes, S. L. Sauter, D. Jolly, T. W. J. Dubensky, P. D. Staber, J. A. Chiorini and B. L. Davidson. Transduction of murine cerebellar neurons with recombinant FIV and AAV5 vectors. NeuroReport (2000) 11:2669-2673.
Anderson G D, Hauser S D, McGarity K L, Bremer M E, Isakson P C and Gregory S A (1996). Selective inhibition of cyclooxygenase (COX)-2 reverses inflammation and expression of COX-2 and interleukin 6 in rat adjuvant arthritis. J Clin Invest 97:2672-79.
Arend, W. P. The balance between IL-1 and IL-1RA in disease. Cytokine & Growth Factor Rev. (2002) 13:323-340.
Balkhi K M, Tallents R H, Hutta J, Murphy W, Proskin H (1993). Electromyographic Evaluation of the Reproducibility of the Activity of the Anterior Temporalis and Masseter with Deliberate Unilateral Chewing. J Orofacial Pain 7:89-97.
Basu, A., J. K. Krady, M. O'Malley, S. D. Styren, S. T. DeKosky and S. W. Levinson. The type 1 interleukin-1 receptor is essential for the efficient activation of microglia and the induction of multiple proinflammatory mediators in response to brain injury. J. Neurosci. (2002) 22:6071-6082.
Bellinger D L, Felten D L, Lorton D. Brouxhon S M (2001). Effects of interleukin-2 on the expression of corticotropin-releasing hormone in nerves and lymphoid cells in secondary lymphoid organs from the Fischer 344 rat. J Neuroimmunol 119:37-50.
Björkdahl, O., P. Åkerblad, A. Gjörloff-Wingren, T. Leanderson and M. Dohlsten. Lymphoid hyperplasia in transgenic mice over-expressing a secreted form of the human interleukin-1b gene product. Immunology (1999) 96:128-137.
Blömer, U., L. Naldini, T. Kafri, D. Trono, I. M. Verma and F. H. Gage. Highly efficient and sustained gene transfer in adult neurons with a lentivirus vector. J. Virol. (1997) 71:6641-6649.
Brenner, M., W. C. Kisseberth, Y. Su, F. Besnard and A. Messing. GFAP promoter directs astrocyte-specific expression in transgenic animals. J. Neurosci. (1994) 14:1030-1037.
Breyer R M, Bagdassarian C K, Myers S A and Breyer M D (2001). Prostanoid receptors: subtypes and signaling. Ann Rev Pharmacol Toxicol 41:661-90.
Brooks A I, Halternan M W, Federoff H J (1999). Focal hippocampal gain of NGF function elicits specific septal cholinergic reorganization. Neuroreport. 10:337-44.
Brooks A I, Muhkerjee B, Panahian N, Cory-Slechta D, Federoff H J (1997). Nerve growth factor somatic mosaicism produced by herpes virus-directed expression of cre recombinase. Nat Biotech 15(1):57-62
Brooks, A. I., B. Muhkerjee, N. Panahian, D. Cory-Slechta and H. J. Federoff. Nerve growth factor somatic mosaicism produced by herpes-simplex virus-directed expression of cre recombinase. Nature Biotech. (1997) 15:57-62.
Brooks, A. I., C. S. Stein, S. M. Hughes, J. Heth, P. M. J. McCray, S. L. Sauter, J. C. Johnston, D. A. Cory-Slechta, H. J. Federoff and B. L. Davidson. Functional correction of established central nervous system deficits in an animal model of lysosomal storage disease with feline immunodeficiency virus-based vectors. Proc. Natl. Acad. Sci. USA (2002) 99:5760-5762.
Brooks, A. I., D. A. Cory-Slechta and H. J. Federoff. Gene-experience interaction alters the cholinergic septohippocampal pathway of mice. Proc. Natl. Acad. Sci. USA (2000) 97:13378-13383.
Brooks, A. I., M. W. Halternan and H. J. Federoff. Focal hippocampal gain of NGF function elicits specific septal cholinergic reorganization. NeuroReport (1999) 10:337-344.
Brooks, A. I., M. W. Halternan, C. A. Chadwick, B. L. Davidson, M. Haak-Frendscho, C. Radel, C. Porter and H. J. Federoff. Reproducible and efficient murine CNS gene delivery using a microprocessor-controlled injector. J. Neurosci. Meth. (1998) 80:137-147.
Brouxhon S M, Prasad A V, Joseph S A, Felten D L, Bellinger D L (1998). Localization of CRF in primary and secondary lymphoid organs of the rat. Brain Beh Immun 12: 107-22.
Brugg, B., Y. L. Dubreuil, G. Huber, E. E. Wollman, N. Delhaye-Bouchard and J. Mariana. Inflammatory processes induce 13-amyloid precursor protein changes in mouse brain. Proc. Natl. Acad. Sci. USA (1995) 92:3032-3035.
Bullough P G, DiCarlo E F (1990). Subchondral avascular necrosis: a common cause of arthritis. Ann Rheum Dis 49:412-20
Burkhardt H, Schwingel M, Menninger H, Macartney H W, Tschesche H (1986). Oxygen radicals as effectors of cartilage destruction. Direct degradative effect on matrix components and indirect action via activation of latent collagenase from polymorphonuclear leukocytes. Arthritis Rheum 29:379-87.
Burns, J. C., T. Friedmann, W. Driever, M. Burrascano and J. K. Yee. Vesicular stomatitis virus G glycoprotein pseudotyped retroviral vectors: concentration to very high titer and efficient gene transfer into mammalian and nonmammalian cells. Proc. Natl. Acad. Sci. USA 90:8033-8037. 1993
Calvelou P, Dallel R, Orliaguet T, Woda A (1995). The orofacial formalin test in rats: effects of different formalin concentrations. Pain 62:295-301.
Campbell, I. L., C. R. Abraham, E. Masliah, P. Kemper, J. D. Inglis, M. B. A. Oldstone and L. Mucke. Neurologic disease induced in transgenic mice by cerebral overexpression of interleukin 6. Proc. Natl. Acad. Sci. USA (1993) 90:10061-10065
Carleson J, Alstergren P, Appelgren A, Appelgren B, Kopp S, Theodorsson E, et al. (1996). A model for the study of experimentally induced temporomandibular arthritis in rats: the effect of human recombinant interleukin-1 alpha on neuropeptide like immunoreactivity. J Orofac Pain 10:9-14.
Chandrasekharan N V, Dai H, Roos K L, Evanson N K, Tomsik J, Elton T S, Simmons D L (2002). COX-3, a cyclooxygenase-1 variant inhibited by acetaminophen and other analgesic/antipyretic drugs: cloning, structure, and expression. Proc Natl Acad Sci USA. 99:13926-31.
Chang J S, Gillman S C, Lewis A J (1986). Interleukin 1 activates phospholipase A2 in rabbit chondrocytes: a possible signal for IL-1 action. J Immunol 136:1283-87.
Chang, J. W., P. D. Coleman and M. K. O'Banion. Prostaglandin G/H synthase-2 (cyclooxygenase-2) mRNA expression is decreased in Alzheimer's disease. Neurobiol. Aging (1996) 17:801-808.
Choi H S, Lee H J, Juan C Y, Ju J S, Park J S, Ahn D K (2003). Central cyclooxygenase-2 participates in interleukin-1b-induced hyperalgesia in the orofacial formalin test of freely moving rats. Neurosci Letters 352: 187-.
Cotman, C. W., A. J. Tenner and B. J. Cummings. 3-amyloid converts an acute phase injury response to chronic injury responses. Neurobiol. Aging (1996) 17:723-731.
Crombie I K, Croft P R, Linton S J, LeResche L, Von Korff M (eds). Epidemiology of Pain: A report of the Task Force on epidemiology of the International Association for the Study of Pain. Seattle: IASP, 1999.
Daly T M, Vogler C, Levy B, Haskins M E, Sands M S (1999) Neonatal gene transfer leads to widespread correction of pathology in a murine model of lysosomal disease. Proc Natl Acad Sci USA 96: 2296-3000.
de Bont L G, Boering G, Liem R S, Eulderink F, Westesson P L (1986). Osteoarthritis and internal derangement of the temporomandibular joint: a light microscopic study. J Oral & Maxillofac Surg 44:634-43.
Dubuisson D, Dennis S. The formalin test: A quantitative study of the analgesic effects of morphine, meperidine, and brainstem stimulation in rats and cats. Pain 1977; 4: 161-74.
Dijkgraaf L C, de Bont L G, Boering G, Liem R S (1995). The structure, biochemistry, and metabolism of osteoarthritic cartilage: a review of the literature. J Oral Maxillofac Surg 53:1182-92.
Dijkgraaf L C. Liem R S. de Bont L G (1997). Synovial membrane involvement in osteoarthritic temporomandibular joints: a light microscopic study. Oral Surg Oral Med Oral Pathol Oral Radiol & Endodont 83:373-86.
Dijkgraaf L C. Spijkervet F K. de Bont L G (1999). Arthroscopic findings in osteoarthritic temporomandibular joints. J Oral & Maxillofac Surg 57:255-68.
Dinchuk J E, Liu R Q, Trzaskos J M (2003) COX-3: in the wrong frame in mind. Immunol Lett. 86:121.
Du, Y., R. C. Dodel, B. J. Eastwood, K. R. Bales, F. Gao, F. Lohmuller, U. Muller, A. Kurz, R. Zimmer, R. M. Evans, A. Hake, T. Gasser, W. H. Oertel, W. S. Griffin, S. M. Paul and M. R. Farlow. Association of an interleukin 1 alpha polymorphism with Alzheimer's disease. [see comments]. Neurology (2000) 55:480-483.
Fernandes J C, Caron J P, Martel-Pelletier J, Javanovic D, Mineau F, Tardif G, Otterness I G, Pelletier J P (1997). Effects of tenidap on the progression of osteoarthritic lesions in a canine experimental model. Suppression of metalloprotease and interleukin-1 activity. Arthritis & Rheum 40:284-94.
Fiorucci S, Meli R, Bucci M, Cirino G (2001) Dual inhibitors of cycloxygenase and 5-lipoxygenase. A new avenue in anti-inflammatory therapy? Biochem Pharmacol 62:1433-8.
Fitzgerald G A and Patrono C (2001). The coxibs, selective inhibitors of cyclooxygenase-2. N Engl J Med 345:433-42.
Frautschy, S. A., F. Yang, M. Irrizarry, B. Hyman, T. C. Saido, K. Hsiao and G. M. Cole. Microglial response to amyloid plaques in APPsw transgenic mice. Am. J. Pathol. (1998) 152:307-317.
Friedlander, R. M., V. Gagliardini, N. Hara, K. B. Fink, W. Li, G. MacDonald, M. C. Fishman, A. H. Greenberg, M. A. Moskowitz and J. Yuan. Expression of a dominant negative mutant o interleukin-1b converting enzyme in transgenic mice prevents neuronal cell death induced by trophic factor withdrawal and ischemic brain injury. J. Exp. Med. (1997) 185:933-940.
Frisella, W. A., L. H. O'Connor, C. A. Vogler, M. Roberts, S. Walkley, B. Levy, T. M. Daly and M. S. Sands. Intracranial injection of recombinant adeno-associated virus improves cognitive function in a murine model of mucopolysaccharidosis type VII. Mol. Therapy. (2001) 3:351-358.
Futaki N, Arai I, Hamasaka Y, Takahashi S, Higuchi S and Otomo S (1993). Selective inhibition of NS-398 on prostanoid production in inflamed tissue in rat carrageenan-air-pouch inflammation. J Pharmacol 45:753-55.
Gillman S C, Chang J, Zeigler P R, Uhl J, Mochan E (1988). Interleukin 1 activates phospholipase M in human synovial cells. Arthritis Rheum 31:126-30.
Gilroy D W, Colville-Nash P R, McMaster S, Sawatzky D A, Willoughby D A, Lawrence T (2003). Inducible cyclooxygenase-derived 15-deoxy(Delta)12-14PGJ2 brings about acute inflammatory resolution in rat pleurisy by inducing neutrophil and macrophage apoptosis. FASEB 17:2269-71.
Gilroy D W, Colville-Nash P R, Willis D, Chivers J, Paul-Clark M J, Willoughby D A (1999). Inducible cyclooxygenase may have anti-inflammatory properties. Nat Med 5:698-701.
Gjörloff-Wingren, A., O. Björkdahl, T. Labuda, L. Björk, U. Andersson, U. Gullberg, G. Hedlund, H.-O, Sjögren, T. Kalland, B. Widegren and M. Dohlsten. Fusion of a signal sequence to the interleukin-1b gene directs the protein from cytoplasmic accumulation to extracellular release. Cell. Immunol. (1996) 169:226-237.
Goldgaber, D., H. H. Harris, T. Hla, T. Maciag, R. J. Donnelly, J. S. Jacobsen, M. P. Vitek and D. C. Gajdusek. Interleukin 1 regulates synthesis of amyloid β-protein precursor mRNA in human endothelial cells. Proc. Natl. Acad. Sci. USA (1989) 86:7606-7610.
Gray, C. W. and A. J. Patel. Regulation of β-amyloid precursor protein isoform mRNAs by transforming growth factor-β1 and interleukin-1β in astroctyes. Mol. Brain. Res. (1993) 19:251-256.
Greenwald R A, Moy W W (1997). Inhibition of collagen gelation by action of the superoxide radical. Arthritis Rheum 22:251-59.
Griffin, W. S., J. A. Nicoll, L. M. Grimaldi, J. G. Sheng and R. E. Mrak. The pervasiveness of interleukin-1 in alzheimer pathogenesis: a role for specific polymorphisms in disease risk. Exp Gerontol (2000) 35:481-487.
Griffin, W. S., J. G. Sheng, M. C. Royston, S. M. Gentleman, J. E. McKenzie, D. I. Graham, G. W. Roberts and R. E. Mrak. Glial-neuronal interactions in Alzheimer's disease: the potential role of a ‘cytokine cycle’ in disease progression. Brain Pathol. (1998) 8:65-72.
Griffin, W. S. T., J. G. Sheng and R. E. Mrak. Inflammatory pathways: implications in Alzheimer's disease, in: (Ed.) Wasco, W., R. E. Tanzi, Molecular Mechanisms of Dementia, Humana Press, Inc., Totowa, N.J., (1997) pp. 169-176.
Griffin, W. S. T., J. G. Sheng, G. W. Roberts and R. E. Mrak. Interleukin-1 expression in different plaque types in Alzheimer's disease: significance in plaque evolution. J. Neuropath. Exp. Neurol. (1995) 54:276-281.
Griffin, W. S. T., L. C. Stanley, C. Ling, L. White, V. MacLeod, L. J. Perrot, C. L. White, III and C. Araoz. Brain interleukin 1 and S-100 immunoreactivity are elevated in Down syndrome and Alzheimer disease. Proc. Natl. Acad. Sci. USA (1989) 86:7611-7615.
Grilli, M., F. Goffi, M. Memo and P. Spano. Interleukin-1β and glutamate activate the NF-kappaB/Re1 binding site form the regulatory region of the amyloid precursor protein gene in primary neuronal cultures. J. Biol. Chem. (1996) 271:15002-15007.
Grimaldi, L., V. Casadei, C. Ferri, F. Veglia, F. Licastro, G. Annoni, I. Biunno, G. De Bellis, S. Sorbi, C. Mariani, N. Canal, W. Griffin and M. Franceschi. Association of early-onset Alzheimer's disease with an interleukin-1 alpha gene polymorphism. Ann. Neurol. (2000) 47:361-365.
Han R, Tsui S and Smith T J (2002). Up-regulation of prostaglandin E2 synthesis by interleukin-1β in human orbital fibroblasts involves coordinate induction of prostaglandin H synthase-2 and glutathione-dependent prostaglandin E2 synthase expression. J Biol Chem 277:163555-64.
Hart R P, Shadiack A M, Jonakait G M (1991). Substance P gene expression is regulated by interleukin-1 in cultured sympathetic ganglia. J Neurosci Res 29:282-86.
Havenga M J, Vogels R, Braakman E, Kroos N, Valerio D, Hagenbeek A, van Es H H (1998). Second gene expression in bicistronic constructs using short synthetic intercistrons and viral IRES sequences. Gene. 222:319-27.
Helkimo E, Carlsson G E, Yehuda C (1975). Bite force in patients with functional disturbances of the masticatory system. J Oral Rehabil 2:397-406.
Helminen H J, Kiraly K, Pelttari A, Tammi M, Vandenberg P, Pereira R, et al. (1993). An inbred line of transgenic mice expressing an internally deleted gene for type 1 procollagen (COL2A1). J Clin Invest 92:582-95.
Hoshino T. Senior Thesis (1998), Division of Orthodontics, Eastman Department of Dentistry, University of Rochester School of Medicine and Dentistry.
Rochester, N.Y.
Hsiao, K., P. Chapman, S, Nilsen, C. Eckman, Y. Harigaya, S. Younkin, F. Yang and G. Cole. Correlative memory deficits, A13 elevation and amyloid plaques in transgenic mice. Science (1996) 274:99-102.
Huang, D., R. Pirskanen, P. Hjelmstrom and A. K. Lefvert. Polymorphisms in IL-1 beta and IL-1 receptor antagonist genes are associated with myasthenia gravis. J. Neuroimmunol. (1998) 81:76-81.
Hutchins B, Patel H, Spears R (2002). Attenuation of pro-inflammatory neuropeptide levels produced by a cyclooxygenase-2 inhibitor in an animal model of chronic temporomandibular joint inflammation. J Orofac Pain 16:312-6.
Ichikawa H, Wakisaka S, Matsuo S, et al. (1989). Peptidergic innervation of the temporomandibular disk in the rat. Experientia 45:303-08.
Irwin, M. H., W. D. Pogozelski, and C A Pinkert (2002). PCR optimization for detection of transgene integration, p. 475-484. In C. A. Pinkert (ed.), Transgenic animal technology: a laboratory handbook. 2nd ed. Academic Press, Inc., San Diego.
Israel H A, Diamond B E, Saed-Nejad F, Ratcliffe A (1997). Correlation between arthroscopic diagnosis of osteoarthritis and synovitis of the human temporomandibular joint and keratan sulfate levels in the synovial fluid. J Oral Maxillofacial Surg 55:210-7.
Israel H A, Saed-Nejad F, Ratcliffe A (1991). Early diagnosis of osteoarthrosis of the temporomandibular joint: correlation between arthroscopic diagnosis and keratan sulfate levels in the synovial fluid. J Oral Maxillofacial Surg 49:708-11.
Jakobsson P J, Thorén S, Morgenstern R, Samuelsson B (1999). Identification of human prostaglandin E synthase: a microsomal, glutathione-dependent, inducible enzyme, constituting a potential novel drug target. Proc Natl Acad Sci USA 96:7220-25.
Jantzen P T, Connor K E, DiCarlo G, Wenk G L, Wallace J L, Rojiani A M, Coppola D, Morgan D, Gordon M N (2002). Microglial Activation and b-Amyloid Deposit Reduction Caused by a Nitric Oxide-Releasing Nonsteroidal Anti-Inflammatory Drug in Amyloid Precursor Protein Plus Presenilin-1 Transgenic Mice. J Neurosci 22:2246-.
Jiao Y, Ma X, Zhang Z (2001). Interleukin-1 increase nitric oxide synthesis through up-regulation of inducible nitric-oxide synthase by rabbit mandibular condylar cartilage cells in vitro. Chin J Stomatol 36:345-7.
Johansson A-S, Isacson G, Isberg A, et al. (1986) Distribution of substance P-like immunoreactive nerve fibers in temporomandibular joint soft tissues of monkey. Scand J Dent Res 94:225-30.
Jonakait G M, Schotland S (1990). Conditioned medium from activated splenocytes increases substance P in sympathetic ganglia. J Neurosci Res 26:24-30.
Jonakait G M, Schotland S, Hart R P (1991). Effects of lymphokines on substance P in injured ganglia of the peripheral nervous system. Ann NY Acad Sci 632:19-30.
Jonakait G M, Schotland S, Hart R P (1991). Interleukin-1 specifically increases substance P in injured sympathetic ganglia. Ann NY Acad Sci 594:222-30.
Jovanovic D V, Fernandes J C, Martel-Pelletier J, Jolicoeur F C, Reboul P, Laufer S, Tries S, Pelletier J P (2001). In vivo dual inhibition of cyclooxygenase and lipoxygenase by ML-3000 reduces the progression of experimental osteoarthritis: suppression of collagenase 1 and interleukin-1 beta synthesis. Arthr Rheumaton 44:2320-30.
Jow R W and Clark G T (1989). Endurance and recovery from a sustained isometric contraction of human jaw elevating muscles. Arch Oral Biol 34: 857-62.
Kang Y, Stein C S, Heth J A, Sinn P L, Penisten A K, Staber P D, Ratliff K L, Shen H, Barker C K, Martins I, Sharkey C M, Sanders D A, McCray P B Jr., Davidson B L (2002). In vivo gene transfer using a nonprimate lentiviral vector pseudotyped with ross river virus glycoproteins. J Virol 76 9378-88.
Kawai Y, Kubota, Okabe E (2000). Reactive oxygen species participation in experimentally induced arthritis of the temporomandibular joint in rats. J Dent Res 79:1489-95.
Kawakami M, Okabe E (1998). Superoxide anion radical-triggered Ca+2 release from cardiac sarcoplasmic reticulum through ryanodine receptor Ca+2 channel. Mol Pharmacol 53:497-503.
Kehl L J, Trempea T M and Hargreaves K M (2000). A new animal model for assessing mechanisms and management of muscle hyperalgesia. Pain 85:333-43.
Kido M A, Kiyoshima T, Kondo T, et al. (1993) Distribution of substance P and calcitonin gene-related peptide-like immunoreactive nerve fibers in the rat temporomandibular joint. J Dent Res 72:592-98.
Kimmich, G. A., J. Roussie, M. Manglapus and J. Randles. Characterization of Na+-coupled glutamate/aspartate transport by a rat brain astrocyte line expressing GLAST and EAAC1. J. Membr. Biol. (2001) 182:17-30.
Kirtikara K, S G Morham, R Raghow, S J Laulederkind, T Kanekura, S Goorhaand, L R Ballou (1998). Compensatory prostaglandin E2 biosynthesis in cyclooxygenase 1 or 2 null cells. J Exp Med 187:517-23.
Kis B, Snipes J A, Isse T, Nagy K, Busija D W. (2003) Putative cyclooxygenase-3 expression in rat brain cells. J Cereb Blood Flow Metab. 23: 1287-92.
Kitanaka J, Hashimoto H, Gotoh M, Kondo K, Sakata K, Hirasawa Y, Sawada M, Suzumura A, Marunouchi T, Matsuda T and Baba A. (1996) Expression pattern of messenger RNAs for prostanoid receptors in glial cell cultures. Brain Res 707:282-87.
Kornman, K. S., A. Crane, H. Y. Wang, F. S. di Giovine, M. G. Newman, F. W. Pirk, T. G. Wilson, Jr., F. L. Higginbottom and G. W. Duff. The interleukin-1 genotype as a severity factor in adult periodontal disease. J. Clin. Periodontol. (1997) 24:72-77.
Krebsbach P H, Harrison J R, Lichtler A C, Woody C O, Rowe D W, Kream B E (1993). Transgenic expression of COL1A1-chloramphenicol acetyltransferase fusion genes in bone: differential utilization of promoter elements in vivo and in cultured cells. Mol Cell Biol 13: 5168-74.
Kubota E, Kubota T, Matsumoto J, Shibata T, Murakami Kl (1998). Synovial fluid cytokines and proteinases as markers of temporomandibular joint disease. J Oral Maxillofac Surg 56:192-98.
Kyrkanides S, Kambylafkas P, Miller J H, Tallents R H (2004). Non-primate lentiviral vector administration in the TMJ. J Dental Res 83: 65-70.
Kyrkanides S, Miller J H, Bowers W A, Federoff H J (2003). Transcriptional and post-translational regulation of Cre recombinase by RU486 as the basis for an enhanced inducible expression system. Mol Ther 8: 790-95.
Kyrkanides S, Miller J H, Federoff H J (2003). Systemic FIV vector administration: Transduction of CNS immune cells and Purkinje neurons. Mol Brain Res 119:1-9.
Kyrkanides S, Moore A H, Olschowka J A, Williams J P, Hansen J T, O'Banion M K (2002). COX-2 modulates inflammation related genes in CNS radiation injury. Brain Res. Mol Brain Res 104: 159-69.
Kyrkanides S, O'Banion M K, Subtelny J D (2000). Non-steroidal anti-inflammatory drugs in orthodontic tooth movement: Metalloproteinase activity and collagen synthesis by endothelial cells. Am J Orthod Dentofac Orthop 118: 203-09.
Kyrkanides S, Olschowka J A, Whitley P, O'Banion M K (2001). Enhanced glial activation and expression of specific CNS inflammation-related molecules in aged versus young rats following cortical stab injury. J Neuroimmunol 119: 269-77.
Kyrkanides S, Olschowka J A, Williams J P, Hansen J T, O'Banion M K (1999). TNFα & IL-1β mediate ICAM-1 induction via microglia-astrocyte interaction in CNS radiation injury. J Neuroimmunol 95:95-106.
Kyrkanides S, Tallents R H, Macher D J, Olschowka J A, Stevens S Y (2002). Temporomandibular joint nociception: effects of capsaicin on substance P-like immunoreactivity in the rabbit brain stem. J Orofac Pain 16:229-35.
Kyrkanides, S., J. A. Olschowka, J. P. Williams, J. T. Hansen and M. K. O'Banion. TNFα and IL-1β mediate intercellular adhesion molecule-1 induction via microglia-astrocyte interaction in CNS injury. J. Neuroimmunol. (1999) 95:95-106.
Lane N E (1997). Pain management in osteoarthritis: the role of COX-2 inhibitors. J Rheumatol 24 Suppl. 49:20-24.
Langenbach R, Morham S G, Tiano H F, Loftin C D, Ghanayem B I, Chulada P C, Mahler J F, Lee C A, Goulding E H, Kluckman K D, Kim H S, Smithies 0 (1995). Prostaglandin Synthase 1 Gene Disruption in Mice Reduces Arachidonic Acid-Induced Inflammation and Indomethacin-Induced Gastric Ulceration. Cell 83:483-92.
Lavigne P, Shi Q, Jolicoeur F C, Pelletier, Martel-Pelletier J, Feman des J C (2002). Modulation of IL-1 beta, IL-6, TNF-alpha and PGE(2) by pharmacological agents in explants of membranes from failed total hip replacement. Osteoarthritis & Cartilage 10:898-904.
Lehmann, J. M., J. M. Lenhard, B. B. Oliver, G. M. Ringold and S. A. Kleiwer (1997). Peroxisome proliferator-activated receptors alpha and gamma are activated by indomethacin and other non-steroidal anti-inflammatory drugs. J Biol Chem 272:3406-10.
Lee Y C, Lai H L, Sun C N, Chien C L, Chem Y. Identification of nuclear factor 1 (NF1) as a transcriptional modulator of rat A(2A) adenosine receptor. Brain Res Mol Brain Res. 2003 111(1-2):61-73.
Li, Y., L. Liu, S. W. Barger and W. S. Griffin. Interleukin-1 mediates pathological effects of microglia on tau phosphorylation and on synaptophysin synthesis in cortical neurons through a p38-MAPK pathway. J. Neurosci. (2003) 23:1605-1611.
Lipsky P E and Isakson P C (1997). Outcome of specific COX-2 inhibition in rheumatoid arthritis. J Rheumatol 24:9-14.
Lipton J A, Ship J A, Larach-Luchini S, Merskey H (1993). Estimated prevalence and distribution of reported orofacial pain in the United States. JADA 124:115-21.
Liu F, Malaval L, Aubin J E (1997). The mature osteoblasts phenotype is characterized by extensive plasticity. Exp Cell Res 232: 97-105.
Liu, C., Y. Bai, D. Ganea and R. P. Hart. Species-specific activity of rat recombinant interleukin-1 beta. J. Interferon Cytokine Res. (1995) 15:985-992.
Loddick, S. A. and N. J. Rothwell. Neuroprotective effects of human recombinant interleukin-1 receptor antagonist in focal cerebral ischaemia in the rat. J. Cereb. Blood Flow Metab. (1996) 16:932-940.
Lue, L. F., L. Brachova, W. H. Civin and J. Rogers. Inflammation, A beta deposition, and neurofibrillary tangle formation as correlates of Alzheimer's disease neurodegeneration. J. Neuropathol. Exp. Neurol. (1996) 55:1083-1088.
Lund J P, Donga R, Widmer C G, Stohler C S (1991). The pain adaptation model: A discussion of the relationship between chronic musculoskeletal pain and motor activity. Can J Physiol Pharmcol 69:683-694.
Macher D J, Westesson P L, Brooks S L Hicks D and Tallents R H (1992). Temporomandibular joint surgically created disc displacement causes arthrosis in the rabbit. Oral Surg Oral Med Oral Pathol 73:645-49.
Mackenzie, I. R. A. and D. G. Munoz. Nonsteroidal anti-inflammatory drug use and Alzheimer-type pathology in aging. Neurology (1998) 50:986-990.
Maguire-Zeiss K A, Bowers W J, Federoff H J (2002). Somatic mosaic approaches and the aging brain. Neurobiol Aging 23:977-84.
Malmberg A B and Yaksh T L (1995). Cyclooxygenase inhibition and the spinal release of prostaglandin E2 and amino acids evoked by paw formalin injection: a microdialysis study in unanesthetized rats. J Neurosci 15:2768-76.
Mancini J A, Blood K, Guay J, Gordon R, Claveau D, Chan C C and Riendeau R (2001). Cloning, expression, and up-regulation of inducible rat prostaglandin e synthase during lipopolysaccharide-induced pyresis and adjuvant-induced arthritis. J Biol Chem 276:4469-75.
Martel-Pelletier J, Pelletier J P, Fahmi H (2003). Cyclooxygenase-2 and prostaglandins in articular tissues. Semin Arthritis Rheum 33:155-67.
Masaki M, Matsushita M and Wakitani K. Inhibitory effects of JTE-522, a novel prostaglandin H synthase-2 inhibitor, on adjuvant-induced arthritis and bone changes in rats. Inflamm Res 47:187-92.
Matsubara T, Ziff M (1986). Increased superoxide anion release from human endothelial cells in response to cytokines. J Immunol 137:3295-98.
McCord J M (1974). Free radicals and inflammation: protection of synovial fluid by superoxide dismutase. Science 185:529-31.
McDowell, T. L., J. A. Symons, R. Ploski, O. Forre and G. W. Duff. A genetic association between juvenile rheumatoid arthritis and a novel interleukin-1 alpha polymorphism. Arthritis Rheum. (1995) 38:221-228.
Milam S B, Magnuson V L, Steffensen B, Chen D, Klebe R J (1991). Il-1 bata and prostaglandins regulate integrin mRMA expression. J Cell Physiol 149:173-83.
Milam S B, Schmitz J P (1995). Molecular biology of temporomandibular joint disorders: proposed mechanisms of disease. J Oral Maxillofacial Surg 53:1448-54.
Minami, M., K. Kuraishi, K. Yabuuchi, K. Yamazaki and M. Satoh. Induction of interleukin-1 mRNA in rat brain transient forebrain ischaemia. J. Neurochem. (1992) 58:390-392.
Mizukawa H, Okabe E (1997). Inhibition by singlet molecular oxygen of the vascular reactivity in rabbit mesenteric artery. Br J Pharmacol 121:63-70.
Molin C (1972). Vertical isometric muscle forces of the mandible. A comparative study of subjects with and without manifest mandibular pain dysfunction syndrome. Acta Odontol Scand 30:485-99.
Moore A H, Olschowka J A, O'Banion M K (2003). Intraparenchymal administration of interleukin-1b induces cyclooxygenase-2-mediated expression of membrane- and cytosolic-associated prostaglandin E synthases in mouse brain. In Press.
Morham S G, Langenbach R, Loftin C D, Tiano H F, Vouloumanos N, Jennette J C, Mahler J F, Kluckman K D, Ledford A, Lee C A, Smithies 0 (1995). Prostaglandin Synthase 2 Gene Disruption Causes Severe Renal Pathology in the Mouse. Cell 83:472-82.
Mueller-Decker K, Hirschner W, Marks F, Fuerstenberger G (2002). The Effects of Cyclooxygenase Isozyme Inhibition on Incisional Wound Healing in Mouse Skin. J Invest Dermatol 119: 1189-95.
Murakami M, Naraba H, Tanioka T, Semmyo N, Nakatani Y, Kojima F, Ikeda T, Fueki M, Ueno A, Oh-ishi S and Kudo 1 (2000). Regulation of prostaglandin E2 biosynthesis by inducible membrane-associated prostaglandin E2 synthase that acts in concert with cyclooxygenase-2. J Biol Chem 275:32783-92.
Murphy, G. M., Jr., J. D. Claassen, J. J. DeVoss, N. Pascoe, J. Taylor, J. R. Tinklenberg and J. A. Yesavage. Rate of cognitive decline in AD is accelerated by the interleukin-1 alpha-889*1 allele. Neurology (2001) 56:1595-1597.
Murray R and Spiegel S (1988). Theory and Problems of Statistics, 2nd ed. Schaum's Outline Series, McGraw-Hill Publishing, New York, Chapter 5, p110-21.
Myers S L, Flusser D, Brandt K D, Heck D A (1992). Prevalence of cartilage shards in synovium and their association with synovitis in patients with early and endstage osteoarthritis. J Rheumatol 19:1247-51.
Nagy A, Gertsenstein M, Vintersten K, and Behringer R. (2003) Manipulating the Mouse Embryo: A Laboratory Manual. 3rd ed., Cold Spring Harbor Laboratory Press, Cold Spring Harbor, N.Y.
Narumiya A and FitzGerald G A (2001). Genetic and pharmacological analysis of prostanoid receptor function. J Clin Invest 108:25-30.
Narumiya S, Sugimoto Y, Ushikuni F (1999). Prostanoid receptors: structures, properties and functions. Physiol. Rev. 79:1193-1226.
Ngo L, Jay G (2002). Analysis of transgene expression, p. 486-513. In CA Pinkert (ed.), Transgenic animal technology: a laboratory handbook. 2nd ed. Academic Press, Inc., San Diego.
Nicoll, J., R. Mrak, D. Graham, J. Stewart, G. Wilcock, S. MacGowan, M. Esiri, L. Murray, D. Dewar, S. Love, T. Moss and W. Griffin. Association of interleukin-1 gene polymorphisms with Alzheimer's disease. Ann. Neurol. (2000) 47:365-368.
Norisue M, Todoki K, Okabe E (1997). Inhibition by hydroxyl radicals of calcitonin gene-related peptide-mediated neurogenic vasorelaxation in isolated canine lingual artery. J Pharmacol Exp Ther 280:492-500.
Norsdenskiold U M and Grimby G (1993). Grip force in patients with rheumatoid arthritis and fibromyalgia and in healthy subjects: a study with the Grippit instrument. Scand J Rheumatol 22: 14-9.
O'Banion M K (1999). Cyclooxygenase-2: molecular biology, pharmacology, and neurobiology. Crit. Rev Neurobiol 13: 45-82.
O'Banion M K, Sadowski H B, Winn V, Young D A (1991). A serum- and glucocorticoid-regulated 4-kilobase mRNA encodes a cyclooxygenase-related protein. J Biol Chem 266: 23261-7.
O'Banion M K, Winn V D, Young D A (1992). cDNA cloning and functional activity of a glucocorticoid-regulated inflammatory cyclooxygenase. Proc Natl Acad Sci U.S.A. 89:4888-92.
O'Banion, M. K., J. C. Dusel, J. W. Chang, M. D. Kaplan and P. D. Coleman. Interleukin-1β induces prostaglandin G/H synthase-2 (cyclooxygenase-2) in primary murine astrocyte cultures. J. Neurochem. (1996) 66:2532-2540.
O'Byrne E M, Blancuzzi V J, Wilson D E, et al. (1990). Increased intra-articular substance P and prostaglandin E2 following injection of interleukin-1 in rabbits. Int J Tissue React 12:11-4.
Ochi T, Ohkubo Y, Mutoh S (2003). Role of cyclooxygenase-2, but not cyclooxygenase-1, on type II collagen-induced arthritis in DBA/1J mice. Biochem Pharmacol 66:1055-60.
Oddo S, Caccamo A, Shepherd J D, Murphy M P, Golde T E, Kayed R, Metherate R, Mattson M P, Akbari Y, LaFerla F M. Triple-transgenic model of Alzheimer's disease with plaques and tangles: intracellular Abeta and synaptic dysfunction. Neuron. 2003 Jul. 31; 39(3):409-21.
Ogura N, To be M, Sakamaki H, Kujiraoka H, Akiba M, Abiko Y, Nagura H (2002). Interleukin-1 beta induces interleukin-6 mRNA expression and protein production in synovial cells from human temporomandibular joint. J Oral Pathol Med 31:353-60.
Olschowka, J. A., W. J. Bowers, S. D. Hurley, M. A. Mastrangelo and H. J. Federoff. Helper-free HSV-1 amplicons elicit a markedly less robust innate immune response in the CNS. Mol. Therapy. (2003) 7:218-227.
Overbeek P A (2002). DNA microinjection and transgenic animal production, p. 72-112. In C. A. Pinkert (Ed.), Transgenic animal technology: a laboratory handbook. 2nd ed. Academic Press, Inc., San Diego.
Paesani D A, Tallents R H, Murphy W C and Proskin H M (1994). Evaluation of the reproducibility of rest activity of the anterior temporal and masseter muscles in asymptomatic and symptomatic temporomandibular subjects. J Orofacial Pain 8:402-06.
Pfeifer A, Brandon E P, Kootstra Neeltje, Gage F H, Verma I M (2001). Delivery of the Cre recombinase by a self-deleting lentiviral vector: Efficient gene targeting in vivo. Proc Natl Acad Sci U.S.A. 98: 11450-5.
Pinkert C. A. (2002) Transgenic Animal Technology: A Laboratory Handbook 2nd ed., Academic Press, San Diego.
Pinkert C A (2003). Transgenic animal technology: Alternatives in genotyping and phenotyping. Comp Med 53:116-29.
Pociot, F., J. Molvig, L. Wogensen, H. Worsaae and J. Nerup. A TaqI polymorphism in the human interleukin-1 beta (IL-1 beta) gene correlates with IL-1 beta secretion in vitro. Eur. J. Clin. Invest. (1992) 22:396-402.
Poeschla, E. M., F. Wong-Staal and D. J. Looney. Efficient transduction of nondividing human cells by feline immunodeficiency virus lentiviral vectors. Nature Med. (1998) 4:354-357.
Polites H G, Pinkert C A (2002). DNA microinjection and transgenic animal production, p. 15-70. In C. A. Pinkert (ed.), Transgenic animal technology: a laboratory handbook. 2nd ed. Academic Press, Inc., San Diego.
Portanova J P, Zhang Y, Anderson G D, Hauser S D, Masferrer J L, Seibert K, Gregory S A, Isakson P C. (1996) Selective neutralization of prostaglandin E2 blocks inflammation, hyperalgesia, and interleukin 6 production in vivo. J. Exp. Med. 184:883-91.
Ratcliffe A, Billingham M E, Saed-Nejad F, Muir H, Hardingham T E (1992). Increased release of matrix components from articular cartilage in experimental canine osteoarthritis. J Ortho Res 10:350-8.
Ratcliffe A, Doherty M, Maini R N, Hardingham T E (1998). Increased concentrations of proteoglycan components in the synovial fluids of patients with acute but not chronic joint disease. Ann Rheum Dis 47:826-32.
Revell P A, Mayston V, Lalor P, Mapp P (1988). The synovial membrane in osteoarthritis: a histological study including the characterisation of the cellular infiltrate present in inflammatory osteoarthritis using monoclonal antibodies. Ann Rheum Dis 47:300-7.
Ricote M, Li A C, Willson T M, Kelly C J and Glass C K (1998). The peroxisome proliferator-activated receptor-g is a negative regulator of macrophage activation. Nature 391:79-82.
Ritchlin C T, Haas-Smith S A, Li P, Hicks D G, Schwarz E M (2003). Mechanisms of TNFa- and RANKL-mediated osteoclastogenesis and bone resorption in psoriatic arthritis. J Clin Investig 111:821-31.
Roberts C R, Roughley P J, Mort J S (1989). Degradation of human proteoglycan aggregate induced by hydrogen peroxide. Protein fragmentation, amino acid modification and hyaluronic acid cleavage. Biochem J 259:805-11.
Rogers, J., S. Webster, L.-F. Lue, L. Brachova, W. H. Civin, M. Emmerling, B. Shivers, D. Walker and P. McGeer. Inflammation and Alzheimer's disease pathogenesis. Neurobiol. Aging (1996) 17:681-686.
Rogers, J. T., L. M. Leiter, J. McPhee, C. M. Cahill, S. Zhan, H. Potter and L. N. Nilsson. Translation of the Alzheimer amyloid precursor protein mRNA is up-regulated by interleukin-1 through 5′-untranslated region sequences. J. Biol. Chem. (1999) 274:6421-6431.
Rothwell, N. J. and G. N. Luheshi. Interleukin 1 in the brain: biology, pathology and therapeutic target. Trends Neurosci. (2000) 23:618-625.
Salvemini D, Misko T P, Masferrer J L (1993). Nitric oxide activates cycloxygenase enzymes. Proc Natl Acad Sci 90:7240-44.
Schwarz E M, Looney R J, O'Keefe R J (2000). Anti-TNF-alpha therapy as a clinical intervention for periprosthetic osteolysis. Arthr Res 2:165-8.
Sciacca, F. L., C. Ferri, K. Vandenbroeck, F. Veglia, C. Gobbi, F. Martinelli, D. Franciotta, M. Zaffaroni, M. Marrosu, G. Martino, V. Martinelli, G. Comi, N. Canal and L. M. Grimaldi. Relevance of interleukin 1 receptor antagonist intron 2 polymorphism in Italian MS patients. Neurology (1999) 52:1896-1898.
Scott-Burden T, Bosley J P, Rosenstrauch D, Henderson K D, Clubb F J, Eichstaedt H C, Eya K, Gregoric I, Myers T J, Radovancevic B, Frazier O H (2002). Use of autologous auricular chondrocytes for lining artificial surfaces: A feasibility study. Ann Thorac Surg 73: 1528-33.
Sessle B J, Hu J W (1991). Mechanisms of pain arising from articular tissues. Can J Physiol Pharmacol 69: 617 626.
Shaftel S S, Olschowka J A, Hurley S D, Moore A H, O'Banion M K. COX-3: a splice variant of cyclooxygenase-1 in mouse neural tissue and cells. Brain Res Mol Brain Res. 2003 Nov. 26; 119(2):213-5. Erratum in: Brain Res Mol Brain Res. 2004 Apr. 7; 123(1-2):136
Sheng, J. G., R. E. Mrak and W. S. Griffin. Glial-neuronal interactions in Alzheimer disease: progressive association of IL-1 alpha+microglia and S100 beta+astrocytes with neurofibrillary tangle stages. J. Neuropathol. Exp. Neurol. (1997) 56:285-290.
Sheng, J. G., S. G. Zhu, R. A. Jones, W. S. Griffin and R. E. Mrak. Interleukin-1 promotes expression and phosphorylation of neurofilament and tau proteins in vivo. Exp. Neurol. (2000) 163:388-391.
Siqueira-Junior J M, Peters R R, Brum-Femandes A J, Ribeiro-do-Valle R M (2003). Effects of valeryl salicylate, a COX-1 inhibitor, on models of acute inflammation in mice. Pharmacol Res 48:437-43.
Smith, W L, DeWitt D L, Garavito R M (2000). cyclooxygenase: structural, cellular, and molecular biology. Annu Rev Biochem 69:145-82.
Srinivas S, Watanabe T, Lin C-S, Williams C, Tanabe Y, Jessell T, Costaniti F (2002). Cre reporter strains produced by targeted insertion of EYFP and ECFP into the ROSA26 locus. BMC Develop Biol 1:4-11.
St Pierre, B. A., J. E. Merrill and J. M. Dopp. Effects of cytokines on CNS cells: glia, in: (Ed.) Ransohoff, R. M., E. N. Beveniste, Cytokines and the CNS, CRC Press, Boca Raton, (1996) pp. 151-168.
Stalder, A. K., Carson, M. J., Pagenstecher, A., Asensio, V. C., Kincaid, C., Benedict, M., Powell, H. C., Masliah, E., Campbell, I. L. Late-onset chronic inflammatory encephalopathy in immune-competent and severe combined immune-deficient (SCID) mice with astrocyte-targeted expression of tumor necrosis factor. Am J Pathol, 1998 153(3):767-83.
Stegenga B, de Bont L G, Boering G (1989). Osteoarthrosis as the cause of craniomandibular pain and dysfunction: a unifying concept. J Oral Maxillofacial Surg 47:249-56.
Stegenga B, de Bont L G, Boering G, Van Willigen J D (1991). Tissue responses to degenerative changes in the temporomandibular joint: a review. J Oral Maxillofacial Surg 49:1079-88.
Sternberg N, Hamilton D (1981). Bacteriophage P1 site-specific recombination. Recombination between loxP sites. J Mol Biol 150: 467-86.
Stichtenoth D O, Thoren S, Bian H, Peters-Golden M, Jakobsson P J and Crofford L (2001). Microsomal prostaglandin E synthase is regulated by proinflammatory cytokines in primary rheumatoid synovial cells. J. Immunol. (2001) 167:469-74.
Stohler C S (1999). Craniofacial pain and motor function: pathogenesis, clinical correlates, and implications. Crit. Rev Oral Biol Med 10:504-18.
Stohler C S, Ashton-Miller J A, Carlson D S (1998). The effect of muscle pain from the mandibular joint and muscles on the masticatory muscle behavior in man. Arch Oral Biol 33:175-82.
Stohler C S, Yamada Y, Ash M M (1985). Antagonistic muscle stiffness and associated reflex behavior in the pain-dysfunction state. Helv Odontol Acta 29:13-20.
Sugimoto Y, Narumiya S and Ichikawa A (2000). Distribution and function of prostanoid receptors: studies from knockout mice. Prog Lipid Res 39:289-314.
Surnii H, Inoue H, Onoue J, Mori A, Oda T, Tsubokura T (1996). Superoxide dismutase activity in arthropathy: its role and measurement in the joints. Hiroshima J Med Sci 45:51-55.
Suzuki T, Segami N, Nisimura M, Nojima T (2002). Co-expression of interleukin-1 beta and tumor necrosis factor alpha in synovial tissues and synovial fluids of temporomandibular joint with internal derangement: comparison with histological grading of synovial inflammation. J of Oral Pathology Med 31:549-57.
Tallents R H, Macher D J, Rivoli P, Scapino R, Puzas J E, Katzberg R W (1990). An animal model for meniscus displacement in the rabbit. J Craniomandib Disord Facial Oral Pain 4:233-40.
Tanaka A, Hase S, Miyazawa T, Ohno R, Takeuchi K (2002). Role of cyclooxygenase (COX)-1 and COX-2 inhibition in nonsteroidal anti-inflammatory drug-induced intestinal damage in rats: relation to various pathogenic events. J Pharmacol Exp Ther 303: 1248-54.
Tanioka T, Nakatani Y, Semmyo N, Murakami M and Kudo 1 (2000). Molecular identification of cytosolic prostaglandin E2 synthase that is functionally coupled with cyclooxygenase-1 in immediate prostaglandin E2 biosynthesis. J Biol Chem 275:32775-82.
Tanzi, R. E. Alzheimer's disease risk and the interleukin-1 genes. Ann Neurol (2000) 47:283-285.
Tawara T, Shingu M, Nobunaga M, Naono T (1991). Effects of recombinant human IL-1b on production of prostaglandin E2, leukotriene B4, NAG, and superoxide by human synovial cells and chondrocytes. Inflammation 15:145-57.
Tehiranian, R., S. Andell-Jonsson, S. M. Beni, I. Yatsiv, E. Shohami, T. B. Bartfai, J. KLundkvist and K. Iverfeldt. Improved recovery and delayed cytokine induction after closed head injury in mice with central overexpression of the secreted isoform of the interleukin-1 receptor antagonist. J. Neurotrauma (2002) 19:939-951.
Tiku M L, Liesch J B, Robertson F M (1990). Production of hydrogen peroxide by rabbit articular chondrocytes. Enhancement by cytokines. J Ihmunol 145:690-96.
Tilley S L, Coffman T M and Koller B H (2001). Mixed messages: modulation of inflammation and immune response by prostaglandins and thromboxanes. J Clin Invest 108:15-23
Tinkle B T, Jay G (2002). Analysis of transgene integration, p. 459-474. In C A Pinkert (ed.), Transgenic animal technology: a laboratory handbook. 2nd ed. Academic Press, Inc., San Diego.
Vane J R, Bakhle Y S, Botting R M (1998). Cyclooxygenases 1 and 2. Annu. Rev. Pharmcol. Toxicol. 38:97-120.
Wada S, Okabe E (1997). Susceptibility of caffeine and INS(1,4,5)P3 induced contractions to oxidants in permeabilized vascular smooth muscle. Eur J Pharmacol 320:51-59.
Wilhehmi G, Faust R (1976). Suitability of the C57 black mouse as an experimental animal for the study of skeletal changes due to ageing, with special reference to osteo-arthrosis and its response to tribenoside. Pharmacol 14:289-96.
Wingren A G, Bjorkdahl O, Labuda T, Bjork L, Andersson U, Gullberg U, Hedlund G, Sjogren H O, Kalland T, Widegren B, Dohlsten M (1996). Fusion of a signal sequence to the interleukin-1 beta gene directs the protein from cytoplasmic accumulation to extracellular release. Cell Immunol 169:226-37.
Woodruff T, Blake D R, Freeman J, Andrews F J, Salt P, Lunec J (1986). Is chronic synovitis an example of reperfusion injury? Ann Rheum Dis 45:608-11.
Yaksh T L, Dirig D M, Conway C M, Svensson C, Luo Z D, Isakson P C (2001). The acute antihyperalgesic action of nonsteroidal, anti-inflammatory drugs and release of spinal prostaglandin E2 is mediated by the inhibition of constitutive spinal cyclooxygenase-2 (COX-2) but not COX-1. J Neurosci 21:5847-53.
Yamamoto Y, Yin M J, Lin K M and Gaynor R B (1999). Sulindac inhibits activation of the NF—kB pathway. J Biol Chem 274:27307-314.
Yamasaki, Y., N. Matsuura, H. Shozuhara, H. Onodera, Y. Itoyama and K. Kogure. Interleukin-1 as a pathogenetic mediator of ischemic brain damage in rats. Stroke (1995) 26:676-681.
Yang, Y., W. W. Quitschke and G. J. Brewer. Upregulation of amyloid precursor protein gene promoter in rat primary hippocampal neurons by phorbol ester, IL-1, and retinoic acid, but not by reactive oxygen species. Mol. Brain. Res. (1998) 60:40-49.
Yermakova, A. V., J. Rollins, L. M. Callahan, J. Rogers and M. K. O'Banion. Cyclooxygenase-1 in human Alzheimer's and control brain: quantitative analysis of expression by microglia and CA3 hippocampal neurons. J. Neuropathol. Exp. Neurol. (1999) 58:1135-1146.
Yin M J, Yamamoto Y and Gaynor R B (1998). The anti-inflammatory agents aspirin and salicylate inhibit the activity of IkB kinase-β. Nature 396:77-80.
Yoshida H, Fukumura Y, Fujita S, Nishida M, Iizuka T (2002). The expression of cyclooxygenase-2 in human temporomandibular joint samples: an immunohistochemical study. J Oral Rehab 29:1146-52.
Zhang J, S Goorha, R Raghowand, L R Ballou (2002). The tissue-specific, compensatory expression of cyclooxygenase-1 and -2 in transgenic mice. Prostagland Other Lipid Mediat 67:121-35.
Zhu J, Musco M L, Grace M J (1999). Three-color flow cytometry analysis of tricistronic expression of eBFP, eGFP, and eYFP using EMCV-IRES linkages. Cytometry 37: 51-9.
Zhu X, Conklin D, Eisenach J C (2003). Cyclooxygenase-1 in the spinal cord plays an important role in postoperative pain. Pain 104:15-23.

What is claimed is:

1. A composition comprising a nucleic acid sequence comprising an inactivating element, wherein the inactivating element is flanked by recombination sites, a positive transcription regulator sequence, a sequence encoding an inflammation element, and a poly A tail.

2. The composition of claim 1, wherein the nucleic acid further comprises a secretion signal sequence.

3. The composition of claim 2, wherein the nucleic acid further comprises a sequence encoding a marker.

4. The composition of claim 3, wherein the nucleic acid further comprises an IRES sequence between the inflammatory element and the marker.

5. The composition of claim 1, wherein the inflammatory element encodes COX-2.

6. The composition of claim 5, wherein the nucleic acid encoding COX-2 comprises human COX-2.

7. The composition of claim 4, wherein inflammatory element encodes IL-1ra.

8. The composition of claim 7, wherein the nucleic acid encoding IL-ra comprises human IL-ra.

9. The composition of claim 4, wherein the inflammatory element encodes IL-1β.

10. The composition of claim 9, wherein the nucleic acid encoding IL-1β comprises human IL-1β.

11. The composition of claim 4, wherein the inactivating element comprises a termination sequence.

12. The composition of claim 4, wherein the inactivating element comprises a frame shift mutation in a known coding sequence.

13. The composition of claim 4, wherein the positive transcription regulator sequence comprises a CMV promoter.

14. The composition of claim 4, wherein the wherein the positive transcription regulator sequence comprises a COLL1 promoter.

15. The composition of claim 4, wherein the wherein the positive transcription regulator sequence comprises a GFAP promoter.

16. The composition of claim 4, wherein the marker sequence comprises nucleic acids encoding β-galactosidase (lacZ).

17. The composition of claim 4, wherein the marker sequence comprises nucleic acids encoding a fluorochrome.

18. The composition of claim 17, wherein the fluorochrome comprises green fluorescent protein (GFP).

19. A composition comprising a vector, wherein the vector comprises the nucleic acid of claim 4.

20. A composition comprising a cell, wherein the cell comprises the composition of claim 4.

21. A composition comprising a cell, wherein the cell comprises the composition of claim 19.

22. A transgenic animal comprising the composition of claim 4.

23. The transgenic animal of claim 22, wherein the animal comprises the composition of claim 4 in a germline cell.

24. The transgenic animal of claim claim 22, further comprising Cre.

25. The transgenic animal of claim 22, wherein delivery of Cre recombinase to cells within the animal will result in the expression of the inflammatory element within those cells.

26. The transgenic animal of claim 22, wherein delivery of Cre recombinase to the circulation of the animal will result in the expression of the inflammatory element within cells in the brain.

27. The transgenic animal of claim 22, wherein delivery of Cre recombinase to cells within the joint of the animal will result in the expression of the inflammatory element within cells in the brain.

28. The transgenic animal of claim 22, wherein delivery of Cre recombinase to cells within the joint of the animal will result in neuroinflammation.

29. A method of making an excision activated transgenic animal with temporally conditional expression of an inflammatory mediator, comprising administering the composition of claim 4 to a cell, wherein the cell will form the animal.

30. A method of screening/testing the effectiveness of an anti-inflammatory compound on the treatment of inflammatory disorders, comprising administering the compound to the animal of claim 22.

31. The methods of claim 30, wherein inflammation is induced by targeted expression of COX-2.

32. The methods of claim 30, wherein inflammation is induced by targeted expression of IL-1β.

33. The methods of claim 30, wherein inflammation is induced by targeted expression of IL-1ra.

34. A transgenic animal comprising an inflammation element which is selectively expressed in a target tissue.

35. The animal of claim 34, wherein the tissues is a nerve cell, bone cell, or cartilage cell.


SEQUENCE LISTING (76 sequences)

SEQ ID NO: 1 - DNA, 21 bp, Artificial Sequence
  Description of Artificial Sequence/note = synthetic construct
aggagatggctgctgagttgg

SEQ ID NO: 2 - DNA, 21 bp, Artificial Sequence
  Description of Artificial Sequence/note = synthetic construct
aatctgactttctgagttgcc

SEQ ID NO: 3 - DNA, 22 bp, Artificial Sequence
  Description of Artificial Sequence/note = synthetic construct
gcagcctctgttccacatacac

SEQ ID NO: 4 - DNA, 21 bp, Artificial Sequence
  Description of Artificial Sequence/note = synthetic construct
aatctgactttctgagttgcc

SEQ ID NO: 5 - DNA, 20 bp, Artificial Sequence
  Description of Artificial Sequence/note = synthetic construct
acacactctatcactggcac

SEQ ID NO: 6 - DNA, 21 bp, Artificial Sequence
  Description of Artificial Sequence/note = synthetic construct
agattgttgtcagtatctgcc

SEQ ID NO: 7 - DNA, 20 bp, Artificial Sequence
  Description of Artificial Sequence/note = synthetic construct
acgcgtcaccttaatatgcg

SEQ ID NO: 8 - DNA, 21 bp, Artificial Sequence
  Description of Artificial Sequence/note = synthetic construct
agattgttgtcagtatctgcc

SEQ ID NO: 9 - DNA, 24 bp, Artificial Sequence
  Description of Artificial Sequence/note = synthetic construct
gcacctgtacgatcactgaactgc

SEQ ID NO: 10 - DNA, 25 bp, Artificial Sequence
  Description of Artificial Sequence/note = synthetic construct
ctttaggaagacacaaattgcatgg

SEQ ID NO: 11 - DNA, 22 bp, Artificial Sequence
  Description of Artificial Sequence/note = synthetic construct
atggaaatctgcagaggcctcc

SEQ ID NO: 12 - DNA, 25 bp, Artificial Sequence
  Description of Artificial Sequence/note = synthetic construct
ctttaggaagacacaaattgcatgg

SEQ ID NO: 13 - DNA, 22 bp, Artificial Sequence
  Description of Artificial Sequence/note = synthetic construct
atggaaatctgcagaggcctcc

SEQ ID NO: 14 - DNA, 28 bp, Artificial Sequence
  Description of Artificial Sequence/note = synthetic construct
ctactcgtcctcctggaagtagaatttg

SEQ ID NO: 15 - DNA, 21 bp, Artificial Sequence
  Description of Artificial Sequence/note = synthetic construct
tttttccagttccgtttatcc

SEQ ID NO: 16 - DNA, 20 bp, Artificial Sequence
  Description of Artificial Sequence/note = synthetic construct
tttatcgccaatccacatct

SEQ ID NO: 17 - DNA, 24 bp, Artificial Sequence
  Description of Artificial Sequence/note = synthetic construct
gagaaccaagcaacgacaaaatac

SEQ ID NO: 18 - DNA, 25 bp, Artificial Sequence
  Description of Artificial Sequence/note = synthetic construct
gcattagaaacagtccagcccatac

SEQ ID NO: 19 - DNA, 20 bp, Artificial Sequence
  Description of Artificial Sequence/note = synthetic construct
ccgtggggaatgtatgagca

SEQ ID NO: 20 - DNA, 22 bp, Artificial Sequence
  Description of Artificial Sequence/note = synthetic construct
ccaggtcctcgcttatgatctg

SEQ ID NO: 21 - DNA, 20 bp, Artificial Sequence
  Description of Artificial Sequence/note = synthetic construct
accacagtccatgccatcac

SEQ ID NO: 22 - DNA, 20 bp, Artificial Sequence
  Description of Artificial Sequence/note = synthetic construct
tccaccaccctgttgctgta

SEQ ID NO: 23 - DNA, 25 bp, Artificial Sequence
  Description of Artificial Sequence/note = synthetic construct
ctgaccactcgaccaggttctgggt

SEQ ID NO: 24 - DNA, 29 bp, Artificial Sequence
  Description of Artificial Sequence/note = synthetic construct
gtggataacccctcccccagcctagacaa

SEQ ID NO: 25 - DNA, 28 bp, Artificial Sequence
  Description of Artificial Sequence/note = synthetic construct
aagcggccaaagcctggagggtggaaca

SEQ ID NO: 26 - DNA, 655 bp, Artificial Sequence
  Description of Artificial Sequence/note = synthetic construct
cgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaatt

SEQ ID NO: 27 - DNA, 655 bp, Artificial Sequence; identical to SEQ ID NO: 26
  Description of Artificial Sequence/note = synthetic construct

SEQ ID NO: 28 - DNA, 2069 bp, Artificial Sequence
  Description of Artificial Sequence/note = synthetic construct
aattccatacctgcttgatccacatatgaactacagggggacatgatgaggtccagtctaaaggtcactggcaacctctctcaagatctccctcactatgccattattcaggattggggaagatgtggctggagcctaaggggctcttcccttccctatggtgggactcattaggagaacctcagcaagcagtccactgtaagctcaaacaaataccatgtcgctggtatggagtaaggctgttgctatgacaggaactcaggggtcttaactggcttgagcgctgggagggggcagcagccaggccttgtctgtaagctgaagacctggcagtgctgagctggtcaccccccaggacctccttttgtgcccaacgagtgactcaccttgggcatagacataatggtcaggggtgggcacgcagcctgcttcccgctgtgctccaggcctccttcgatgctttccgagaagtctattgagctgggagcttgtactgcacccggggctgacatcctggcatcctgggataaaagcagcccacggggctgcccttgccatatgcctcactggcggcagagaacaaggctctattcagcaagtgccctggagtagacaccagaagcccaagcatgggcagaggaaggcaggggttggggggagcagagctgtctgtgttccagaagcccaaggacacagatggctaaggcgcctgggagggacctgagtggaagagatagatgggcctgaagtctcaagcagcaacagcctcctccccgccattggtgagggtggggtttggtttcccggacctacatatccctcagaggcctggtgtgtaggaatttaaaggaggtaaatctcctgagagaatcaggggtacccaggaagacggggtgttacagaaagactccagcatgcacagccaactcactcaaaactactctgtcaggggctgccgggggccaggctcggggtggggggtgggggggcaaagagaagctggaccagggagaaatggcccactaggctggatatgaggccacagaggggctcaggaaggaagcctgctgtcttaccctattaggatctgcgtgcataccttctgctgtgcactctaaacacacagccagaggctcaagttgaccctggagtcacagagagggctccaaccttagccctccactcctgaactccaggaatgagaagatagagttggagcgattcaggggagaggactctgttgagaatgggggtcacaggaaactgtaatataggttgatcccggaggaagggaataggttcttcaagttcctagcatctcacaggccccagagaaggacagagtggggtggtcctggcttaacaggctctaagaactggaagctgattaccccaccaagctgtcactctctgtctctgtctctgtctctgtgtgtgcgcgctcgtgcacacttatcacacaaatgttcatgtgtgtgcacatagatgagttgacaccagaggtcaacctcaggcactgttgccttggttttctgagagagcatttctctctggacctggaactcgccaattagtgagagccaggaagtctgctgattttcactgcccagcactggagtttacaagtatgcactgtcaacccaggccttttgtattcattctgcagctagaacttgggtgggtcttcatgcttgacaggcaagcaatttatggactaagctgtttcctcggccctctcttgacccatttaccagaaagggggttccttgatcaatggcgaacgcaggctggtgtcccaagaaagccttgactctgggtacagtgacctcagtggggtgagaggagttctccccttagctgggctggggcccagctccaccccctcaggctattcaatgggggtgcttccaggaagtcaggggcagatttagtccaacccgttcctccataaaggccctgacatcccaggagccagcagaggcagggcaggatggagcggagacgcatcacctctgcgcgccgctcctatgcctccgagacggtggtcaggggcctcggtcctagtcgac

SEQ ID NO: 29 - DNA, 3633 bp, Artificial Sequence
  Description of Artificial Sequence/note = synthetic construct
tctagaatatagaagccaaggatttcaagggtttccttttctctcttcttctttttttttctttttctttttcctgagatggagtttccttttgtagccctgactgtcctggaattcactctatagaccaggctagcctcacacttagtgatctgcctgcctctgcctcttgggtgcctcaggattcaaggcatgaaccaccactacccgaccagggatttcttacacacttctgactggactaaccaggaaagcagagagggagacaggaagaaaatgctcagaaggaaggagtaggattggaggtgagctgggggaacccagactgagccgtgcagaagacaaggaagaagaaagccacccacacacctaggatccacccacagattttgctctgggtacccctgtctggagactgtagggctttgtgatggagggtggggtagtcttcatgccccgtgccctttactccagacctaaatgcccacccccacatacagctgctcgctctctctctcccctgcccttctcccaagagaccagttctccatccctggtctgcagccaaggctgggggcagaagaactttctggaggatttgagtgagaaaagcaagagagcctcaagtagggactggaacctctgggaagggagtgcagaggagacccgggtatgtgccctacctggtacatttatacctgggcagcctctgctcctgttccagacttcagagcccagacgggtcctctccctccctcatgaggggaaacatttggggaaatttggagagagacagaactcagagctcagcactttcctctttctgtttttcttcttgaggaattttttcccccaactgctgatgactttaccattcttgggggtgggggggtggagattctggcttttgctccccctacactccaagtgccggacaaagccctacattccacaagaagccagggcttcagagtttcctaaagatgaggtggcgtggcgagtctcctccctctcccagctccaactccccctcccccagtctccagccctagcctggccagggaggccccgccaggctgggaggagaccccaagcacattcttcctctcgctgtcatgctgcagaaattaaagacacatctctgagctgggtacccgccaatcgtttcaagttgagaagtggcagaggaggtcccgagcttcagctcatgccacgtgtaaaggaagcttggaacccactgcccacaactcctggggcaaaaacctggagtcagacatggggtgaaggctgtcacacggcacagacacgtcaagcaccccccccaattctagtagtctcctagcctccaccagaaccccagacccttgatgtggcagtcaccagtccacacctgttaggctcttgtctcttcttccagatgagcctggggggcgtgggggtgctagatcaggagcagggaaaagtagctttggataagtgcttttcccaatacaaaacccaacaaagagtgggcagatcacactgtgtagtgcttcgtggaaccctaccctagacaactgccttgaacacctattccctctgatgtacaccatccccgtccactgttagggagtgggcatcctttggaactgaccactgtggaaggcaggactttactgagttccggaactaccatctcagcttctcagccccagccttaccctacaggcactggcataggcgggggcagatcctgggccacaagtcactgccacatggttgggataattgatgaagtcctgtccttccattgctgtctccagttctgcttctctggaaactctatatttttccctttaattatagcctctgcagtctccctctgccaccccacccgcaccgcttagcctaactgcccacggccagcgacgtggctccctccccttctgctcccttggtcttttttatttttttttctttgccttcgttgcacaaaactagctcagggagggcgtgaagggggggggagcaatggaatcttggatggtttggaggaggcgggactccttgcttccacgtttacagctctgaagacggctgtgggggaagtgatacaggacgtctatgggccctgagaggagacccctatgcttccctgccacccacacagtttaacaaaatgaagttcctaagtagagtgggggtcaggcagagcacctttgcagggttgatgggagcccagggaaagaaaggacactgtcttttagggacacatttaaatataagccacttttcttgggggacgacaaatgaccctttcctgattgcagaggtggggaacaatggctgagattttcagcaaagaagcgaggacatgaggagtagccttcaaataaagtcactcagctaccaaaaacaagtttctgccacacaccgagttacctaggtgtccccagaccagatccaagtacagtaaggaaagcaggttctctacagagagaacacggctctatggccaatgccttctacctgctctttctggattgatactgctacctaagagggcctctaaccaattcctggctgtagccacagctgacacaagacctttttctaagacatccctggtcacaggcctcctgtagcaaattccagccctgggatggaggtggtcaggaaagagtttatacaagaagacccaggccacagctttaaggactcagaaacccccctgcccacacggctgcccatcataacgcagaaggtttcttctggaaggacaaggatgtcaaacttctccccaagcctaatcctcagagatgtctccctctgttacacctggggctggagaaaggtgggtctttcatggagccacattcatggcagaacagatagccaccccactcctttcaaacaaccacatatctgactcttagtatctgtgaagagatgtctaatttgttcccaaatattcctaccctgcatacctgggcccacaccatgaggtattctcctccctctaacagtcacatctgcttagctgcctggttcttcggatttggagagatgcttgcctaacttattcttccttaggtcttcccaaggatgccagaaagactatgagacatggccaagaggaccttttcccaattgtgcctgacactgaaccctttgtaatgttccccaactcagattcccaattctacatccttctgatttgaggtcccagaaggaaagtgcaaggggcatcccctacccacaatcagtatatcgaggcccagccacactcagtgatagcacctctggcccatgtagatctgggggacaagggtggcagaattgcaaaggggggagggggctgggtggactcctttcccttcctttccctcctcccccctcttcgttccaaattgggggccgggccaggcagttctgattggctgggggccgggctgctggctccccctctccaagaggcagggttcctcccagccctcctccatcaggatggtataaaaggggcccaggccagtcgtcggagcagacgggagtttcacctccggacggagcaggaggcacacggagtgaggccacgcatgagccgaagctaaccccccaccccagccgcaaagagtctacatgtctagggtctag

SEQ ID NO: 30 - DNA, 1404 bp, Artificial Sequence
  Description of Artificial Sequence/note = synthetic construct
tggtggtggtggacaactaggaaactctggcgctttctcctcccctcacaaaactgagtccagctggagccgcctccagactctctggccagggcctcagagtggtcaacagtccctggccagcgttgctctctccaggctaagggcacccactcccctggagattcctgaacctgggccaggaagagccgaattagacaagtgtctccaatccggctgcgtgcggattttgttgcggtgtccctcggttgtctgcagttcctttagtcccttccctggcctgccccttacacctccacacaggtccccctctgtgtaggaatacaccagaccctctcttagccacacacacctccagtcccccgtctacctagatttttttcatagctagttggatgggggatgggttagggaggctgggtttgcgagcctccaggtgggagttcaccgacaggtactccgcaaaggagctggaaggcaggtctggaaaactgtcccccagatttaggattctgggcagcttccatcagcttatactttggctcccccgcccctaaactccccatccccacctcctttctcccgttacttcgtcctccctcgcctttccagcctcgagtctaaagctccatgcttatgcctctgcaaacaaccccctcccttctaaccccagcagaactccgaggaaaggggccggaggccctcttctcgcctgtggttagagggggcagtgtggcagtcccaagtgggggcgaccggaggccgtctcggtgccccgcccgatcaggccactgggcacatcgggggcgggaagcgggctcaccaaaggggcgactggccttggcaggtgtgggctctggtccgacctgggcaggctccgggggcggggtctcaggttacaacgccacggggggctgggggcggcccgcggtttggttggtttgccagcctttggagcgaccgggagcatataaccggagcctctgctgggagaagcgcagggcgccgctgggctgccgggtctcctgcctcctcctcctgctccgagagcctcctgcatgagggcgaggtagagacccggacccgctccggtctctgccgcctcgccgagcttcgcccgggccaaggctctgcgggcctcgcggtgagccatgattcgcctcggggctccccagtcgctggtgctgctgacgctgctcatcgccacggtcctacaatgtcagggccaggatgcccgtaggtcgcccaccacccctgcctgcttccctgacttgcgacccttctcttcttccctccgtccgagttaggcgccaagtcctaggcgcgtagtgcacaggagaacactgatcctaatcctaattctgctagtgaggagttctgtcgcagcatcctcagtcagagtcg

SEQ ID NO: 31 - DNA, 464 bp, Artificial Sequence
  Description of Artificial Sequence/note = synthetic construct
gcacctgtacgatcactgaactgcacgctccgggactcacagcaaaaaagcttggtgatgtctggtccatatgaactgaaagctctccacctccagggacaggatatggagcaacaagtggtgttctccatgtcctttgtacaaggagaagaaagtaatgacaaaatacctgtggccttgggcctcaaggaaaagaatctgtacctgtcctgcgtgttgaaagatgataagcccactctacagctggagagtgtagatcccaaaaattacccaaagaagaagatggaaaagcgatttgtcttcaacaagatagaaatcaataacaagctggaatttgagtctgcccagttccccaactggtacatcagcacctctcaagcagaaaacatgcccgtcttcctgggagggaccaaaggcggccaggatataactgacttcaccatgcaatttgtgtcttcctaaag

SEQ ID NO: 32 - DNA, 534 bp, Artificial Sequence
  Description of Artificial Sequence/note = synthetic construct
atggaaatctgcagaggcctccgcagtcacctaatcactctcctcctcttcctgttccattcagagacgatctgccgaccctctgggagaaaatccagcaagatgcaagccttcagaatctgggatgttaaccagaagaccttctatctgaggaacaaccaactagttgctggatacttgcaaggaccaaatgtcaatttagaagaaaagatagatgtggtacccattgagcctcatgctctgttcttgggaatccatggagggaagatgtgcctgtcctgtgtcaagtctggtgatgagaccagactccagctggaggcagttaacatcactgacctgagcgagaacagaaagcaggacaagcgcttcgccttcatccgctcagacagcggccccaccaccagttttgagtctgccgcctgccccggttggttcctctgcacagcgatggaagctgaccagcccgtcagcctcaccaatatgcctgacgaaggcgtcatggtcaccaaattctacttccaggaggacgagtag

SEQ ID NO: 33 - DNA, 4465 bp, Artificial Sequence
  Description of Artificial Sequence/note = synthetic construct
caattgtcatacgacttgcagtgagcgtcaggagcacgtccaggaactcctcagcagcgcctccttcagctccacagccagacgccctcagacagcaaagcctacccccgcgccgcgccctgcccgccgctcggatgctcgcccgcgccctgctgctgtgcgcggtcctggcgctcagccatacagcaaatccttgctgttcccacccatgtcaaaaccgaggtgtatgtatgagtgtgggatttgaccagtataagtgcgattgtacccggacaggattctatggagaaaactgctcaacaccggaatttttgacaagaataaaattatttctgaaacccactccaaacacagtgcactacatacttacccacttcaagggattttggaacgttgtgaataacattcccttccttcgaaatgcaattatgagttatgtcttgacatccagatcacatttgattgacagtccaccaacttacaatgctgactatggctacaaaagctgggaagccttctctaacctctcctattatactagagcccttcctcctgtgcctgatgattgcccgactcccttgggtgtcaaaggtaaaaagcagcttcctgattcaaatgagattgtggaaaaattgcttctaagaagaaagttcatccctgatccccagggctcaaacatgatgtttgcattctttgcccagcacttcacgcatcagtttttcaagacagatcataagcgagggccagctttcaccaacgggctgggccatggggtggacttaaatcatatttacggtgaaactctggctagacagcgtaaactgcgccttttcaaggatggaaaaatgaaatatcagataattgatggagagatgtatcctcccacagtcaaagatactcaggcagagatgatctaccctcctcaagtccctgagcatctacggtttgctgtggggcaggaggtctttggtctggtgcctggtctgatgatgtatgccacaatctggctgcgggaacacaacagagtatgcgatgtgcttaaacaggagcatcctgaatggggtgatgagcagttgttccagacaagcaggctaatactgataggagagactattaagattgtgattgaagattatgtgcaacacttgagtggctatcacttcaaactgaaatttgacccagaactacttttcaacaaacaattccagtaccaaaatcgtattgctgctgaatttaacaccctctatcactggcatccccttctgcctgacacctttcaaattcatgaccagaaatacaactatcaacagtttatctacaacaactctatattgctggaacatggaattacccagtttgttgaatcattcaccaggcaaattgctggcagggttgctggtggtaggaatgttccacccgcagtacagaaagtatcacaggcttccattgaccagagcaggcagatgaaataccagtcttttaatgagtaccgcaaacgctttatgctgaagccctatgaatcatttgaagaacttacaggagaaaaggaaatgtctgcagagttggaagcactctatggtgacatcgatgctgtggagctgtatcctgcccttctggtagaaaagcctcggccagatgccatctttggtgaaaccatggtagaagttggagcaccattctccttgaaaggacttatgggtaatgttatatgttctcctgcctactggaagccaagcacttttggtggagaagtgggttttcaaatcatcaacactgcctcaattcagtctctcatctgcaataacgtgaagggctgtccctttacttcattcagtgttccagatccagagctcattaaaacagtcaccatcaatgcaagttcttcccgctccggactagatgatatcaatcccacagtactactaaaagaacgttcgactgaactgtagaagtctaatgatcatatttatttatttatatgaaccatgtctattaatttaattatttaataatatttatattaaactccttatgttacttaacatcttctgtaacagaagtcagtactcctgttgcggagaaaggagtcatacttgtgaagacttttatgtcactactctaaagattttgctgttgctgttaagtttggaaaacagtttttattctgttttataaaccagagagaaatgagttttgacgtctttttacttgaatttcaacttatattataagaacgaaagtaaagatgtttgaatacttaaacactatcacaagatggcaaaatgctgaaagtttttacactgtcgatgtttccaatgcatcttccatgatgcattagaagtaactaatgtttgaaattttaaagtacttttggttatttttctgtcatcaaacaaaaacaggtatcagtgcattattaaatgaatatttaaattagacattaccagtaatttcatgtctactttttaaaatcagcaatgaaacaataatttgaaatttctaaattcatagggtagaatcacctgtaaaagcttgtttgatttcttaaagttattaaacttgtacatataccaaaaagaagctgtcttggatttaaatctgtaaaatcagatgaaattttactacaattgcttgttaaaatattttataagtgatgttcctttttcaccaagagtataaacctttttagtgtgactgttaaaacttccttttaaatcaaaatgccaaatttattaaggtggtggagccactgcagtgttatctcaaaataagaatattttgttgagatattccagaatttgtttatatggctggtaacatgtaaaatctatatcagcaaaagggtctacctttaaaataagcaataacaaagaagaaaaccaaattattgttcaaatttaggtttaaacttttgaagcaaacttttttttatccttgtgcactgcaggcctggtactcagattttgctatgaggttaatgaagtaccaagctgtgcttgaataacgatatgttttctcagattttctgttgtacagtttaatttagcagtccatatcacattgcaaaagtagcaatgacctcataaaatacctcttcaaaatgcttaaattcatttcacacattaattttatctcagtcttgaagccaattcagtaggtgcattggaatcaagcctggctacctgcatgctgttccttttcttttcttcttttagccattttgctaagagacacagtcttctcatcacttcgtttctcctattttgttttactagttttaagatcagagttcactttctttggactctgcctatattttcttacctgaacttttgcaagttttcaggtaaacctcagctcaggactgctatttagctcctcttaagaagattaaaagagaaaaaaaaaggcccttttaaaaatagtatacacttattttaagtgaaaagcagagaattttatttatagctaattttagctatctgtaaccaagatggatgcaaagaggctagtgcctcagagagaactgtacggggtttgtgactggaaaaagttacgttcccattctaattaatgccctttcttatttaaaaacaaaaccaaatgatatctaagtagttctcagcaataataataatgacgataatacttcttttccacatctcattgtcactgacatttaatggtactgtatattacttaatttattgaagattattatttatgtcttattaggacactatggttataaactgtgtttaagcctacaatcattgatttttttttgttatgtcacaatcagtatattttctttggggttacctctctgaatattatgtaaacaatccaaagaaatgattgtattaagatttgtgaataaatttttagaaatctgattggcatattgagatatttaaggttgaatgtttgtccttaggataggcctatgtgctagcccacaaagaatattgtctcattagcctgaatgtgccataagactgaccttttaaaatgttttgagggatctgtggatgcttcgttaatttgttcagccacaatttattgagaaaatattctgtgtcaagcactgtgggttttaatatttttaaatcaaacgctgattacagataatagtatttatataaataattgaaaaaaattttcttttgggaagagggagaaaatgaaataaatatcattaaagataactcaggagaatcttctttacaattttacgtttagaatgtttaaggttaagaaagaaatagtcaatatgcttgtataaaacactgttcactgttttttttaaaaaaaaaacttgatttgttattaacattgatctgctgacaaaacctgggaatttgggttgtgtatgcgaatgtttcagtgcctcagacaaatgtgtatttaacttatgtaaaagataagtctggaaataaatgtctgtttatttttgtactattta

SEQ ID NO: 34 - DNA, 2610 bp, Artificial Sequence
  Description of Artificial Sequence/note = synthetic construct
gaattctaccgggtaggggaggcgcttttcccaaggcagtctggagcatgcgctttagcagccccgctggcacttggcgctacacaagtggcctctggcctcgcacacattccacatccaccggtagcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcgaaggtcctcccgaggcccggcattctcgcacgcttcaaaagcgcacgtctgccgcgctgttctcctcttcctcatctccgggcctttcgacctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatccgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagcttgcagatctgcgactctagaggatctgcgactctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgcgactctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgcgactctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccccatcaagctgatccggaaccctt

SEQ ID NO: 35 - DNA, 75 bp, Artificial Sequence
  Description of Artificial Sequence/note = synthetic construct
atggaaatctgcagaggcctccgcagtcacctaatcactctcctcctcttcctgttccattcagagacgatctgc

SEQ ID NO: 36 - DNA, 24 bp, Artificial Sequence
  Description of Artificial Sequence/note = synthetic construct
gcacctgtacgatcactgaactgc

SEQ ID NO: 37 - DNA, 25 bp, Artificial Sequence
  Description of Artificial Sequence/note = synthetic construct
ctttaggaagacacaaattgcatgg

SEQ ID NO: 38 - DNA, 36 bp, Artificial Sequence
  Description of Artificial Sequence/note = synthetic construct
ttccattcagagacgatctgcgcaaatgtacgatca

SEQ ID NO: 39 - DNA, 43 bp, Artificial Sequence
  Description of Artificial Sequence/note = synthetic construct
tcacctaatcactctcctcctcttcctgttccattcagagacg

SEQ ID NO: 40 - DNA, 43 bp, Artificial Sequence
  Description of Artificial Sequence/note = synthetic construct
atggaaatctgcagaggcctccgcagtcacctaatcactctcc

SEQ ID NO: 41 - DNA, 22 bp, Artificial Sequence
  Description of Artificial Sequence/note = synthetic construct
atggaaatctgcagaggcctcc

SEQ ID NO: 42 - DNA, 28 bp, Artificial Sequence
  Description of Artificial Sequence/note = synthetic construct
ctactcgtcctcctggaagtagaatttg

SEQ ID NO: 43 - DNA, 21 bp, Artificial Sequence
  Description of Artificial Sequence/note = synthetic construct
aacccgttcctccataaaggc

SEQ ID NO: 44 - DNA, 539 bp, Artificial Sequence
  Description of Artificial Sequence/note = synthetic construct
atggaaatctgcagaggcctccgcagtcacctaatcactctcctcctcttcctgttccattcagagacgatctgcgcacctgtacgatcactgaactgcacgctccgggactcacagcaaaaaagcttggtgatgtctggtccatatgaactgaaagctctccacctccagggacaggatatggagcaacaagtggtgttctccatgtcctttgtacaaggagaagaaagtaatgacaaaatacctgtggccttgggcctcaaggaaaagaatctgtacctgtcctgcgtgttgaaagatgataagcccactctacagctggagagtgtagatcccaaaaattacccaaagaagaagatggaaaagcgatttgtcttcaacaagatagaaatcaataacaagctggaatttgagtctgcccagttccccaactggtacatcagcacctctcaagcagaaaacatgcccgtcttcctgggagggaccaaaggcggccaggatataactgacttcaccatgcaatttgtgtcttcctaaag

SEQ ID NO: 45 - DNA, 534 bp, Artificial Sequence; identical to SEQ ID NO: 32
  Description of Artificial Sequence/note = synthetic construct

SEQ ID NO: 46 - DNA, 34 bp, Artificial Sequence
  Description of Artificial Sequence/note = synthetic construct
ataacttcgtataatgtatgctatacgaagttat

SEQ ID NO: 47 - DNA, 34 bp, Artificial Sequence
  Description of Artificial Sequence/note = synthetic construct
ataacttcgtatagcatacattatacgaagttat

SEQ ID NO: 48 - DNA, 581 bp, Artificial Sequence
  Description of Artificial Sequence/note = synthetic construct
aattccgcccctctccctcccccccccctaacgttactggccgaagccgcttggaataaggccggtgtgcgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataa

SEQ ID NO: 49 - DNA, 1278 bp, Artificial Sequence
  Description of Artificial Sequence/note = synthetic construct
tcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggggtttatatcttcccttctctgttcctccgcagccagccatg

SEQ ID NO: 50 - DNA, 1278 bp, Artificial Sequence
  Description of Artificial Sequence/note = synthetic construct
tcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgcgcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgttgtaccggcggggtttatatcttcccttctctgttcctccgcagccagccatg

SEQ ID NO: 51 - DNA, 1729 bp, Artificial Sequence
  Description of Artificial Sequence/note = synthetic construct
gaattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattc

SEQ ID NO: 52 - DNA, 366 bp, Artificial Sequence
  Description of Artificial Sequence/note = synthetic construct
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggt

SEQ ID NO: 53 - DNA, 1295 bp, Artificial Sequence
  Description of Artificial Sequence/note = synthetic construct
ccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattc

SEQ ID NO: 54 - DNA, 1278 bp, Artificial Sequence; identical to SEQ ID NO: 50
  Description of Artificial Sequence/note = synthetic construct

SEQ ID NO: 55 - DNA, 229 bp, Artificial Sequence
  Description of Artificial Sequence/note = synthetic construct
gtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctc

SEQ ID NO: 56 - DNA, 281 bp, Artificial Sequence
  Description of Artificial Sequence/note = synthetic construct
tggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcag

SEQ ID NO: 57 - DNA, 282 bp, Artificial Sequence
  Description of Artificial Sequence/note = synthetic construct
attatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctc

SEQ ID NO: 58 - DNA, 512 bp, Artificial Sequence
  Description of Artificial Sequence/note = synthetic construct
ttgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctc

SEQ ID NO: 59 - DNA, 308 bp, Artificial Sequence
  Description of Artificial Sequence/note = synthetic construct
tcggcgaagcctcgcgcggccggccaggacgaggagcgccactaggttgaacatccgcacgagccgccgggccaggtctcggacgggctctcgagactcgatctcgtgcatgtcggcggtccgcggtgaggttatagaccatctgctaggcgggtccggggagacaggcacattactggcctcggcgcccagcctaggcgtgtctagagctcgaccgcgcgtccggagcgccattcgaccggcgggtagcgagaagaacgccggagaccgcaggttataacaacgtcatgcataaattaagaatgggc

SEQ ID NO: 60 - DNA, 1848 bp, Artificial Sequence
  Description of Artificial Sequence/note = synthetic construct
ctgcagtgaataataaaatgtgtgtttgtccgaaatacgcgtttgagatttctgtcccgactaaattcatgtcgcgcgatagtggtgtttatcgccgatagagatggcgatattggaaaaatcgatatttgaaaatatggcatattgaaaatgtcgccgatgtgagtttctgtgtaactgatatcgccatttttccaaaagttgatttttgggcatacgcgatatctggcgatacgcttatatcgtttacgggggatggcgatagacgcctttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgcacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccagcgaatctcgggtacgtgttccggacatggggctcttctccggtagcggcggagcttctacatccagccctgctcccatcctcccactcatggtcctcggcagctccttgctcctaacagtggaggccagacttaggcacagcacgatgcccaccaccaccagtgtgcccacaaggccgtggcggtagggtatgtgtctgaaaatgagctc

SEQ ID NO: 61 - DNA, 1176 bp, Artificial Sequence
  Description of Artificial Sequence/note = synthetic construct
cccgggcccagcaccccaaggcggccaacgccaaaactctccctcctcctcttcctcaatctcgctctcgctctttttttttttcgcaaaaggaggggagagggggtaaaaaaatgctgcactgtgcggcgaagccggtgagtgagcggcgcggggccaatcagcgtgcgccgttccgaaagttgccttttatggctcgagcggccgcggcggcgccctataaaacccagcggcgcgacgcgccaccaccgccgagaccgcgtccgccccgcgagcacagagcctcgcctttgccgatccgccgcccgtccacacccgccgccaggtaagcccggccagccgaccggggcatgcggccgcggccccttcgcccgtgcagagccgccgtctgggccgcagcggggggcgcatggggggggaaccggaccgccgtggggggcgcgggagaagcccctgggcctccggagatgggggacaccccacgccagttcggaggcgcgaggccgcgctcgggaggcgcgctccgggggtgccgctctcggggcgggggcaaccggcggggtctttgtctgagccgggctcttgccaatggggatcgcagggtgggcgcggcgtagcccccgccaggcccggtgggggctggggcgccattgccggtgcgcgctggtcctttgggcgctaactgcgtgcgcgctgggaattggcgctaattgcgcgtgcgcgctgggactcaaggcgctaattgcgcgtgcgttctggggcccggggtgccgcggcctgggctggggcgaaggcgggctcggccggaaggggtggggtcgccgcggctcccgggcgcttgcgcgcacttcctgcccgagccgctggccgcccgagggtgtggccgctgcgtgcgcgcgcgccgacccggcgctgtttgaaccgggcggaggcggggctggcgcccggttgggagggggttggggcctggcttcctgccgcgcgccgcggggacgcctccgaccagtgtttgccttttatggtaataacgcggccggcccggcttcctttgtccccaatctgggcgcgcgccggcgccccctggcggcctaaggactcggcgcgccggaagtggccagggcgggggcgacttcggctcacagcgcgcccggctattctcgcagctcaccatggatg

SEQ ID NO: 62 - DNA, 49 bp, Artificial Sequence
  Description of Artificial Sequence/note = synthetic construct
cttctggcgtgtgaccggcggggtttatatcttcccttcccaagcttgg

SEQ ID NO: 63 - DNA, 66 bp, Artificial Sequence
  Description of Artificial Sequence/note = synthetic construct
cttctggcgtgtgaccggcggggtttatatcttcccttctctgttcctccgcagccccaagcttgg

SEQ ID NO: 64 - DNA, 68 bp, Artificial Sequence
  Description of Artificial Sequence/note = synthetic construct
cttctggcgtgtgaccggcggggtttatatcttcccttctctgttcctccgcagccagccaagcttgg

SEQ ID NO: 65 - DNA, 69 bp, Artificial Sequence
  Description of Artificial Sequence/note = synthetic construct
cttctggcgtgtgaccggcggggtttatatcttcccttctctgttcctccgcagccagccatggatgat

SEQ ID NO: 66 - DNA, 1278 bp, Artificial Sequence; identical to SEQ ID NO: 50
  Description of Artificial Sequence/note = synthetic construct

SEQ ID NO: 67 - DNA, 1176 bp, Artificial Sequence; identical to SEQ ID NO: 61
  Description of Artificial Sequence/note = synthetic construct

SEQ ID NO: 68 - DNA, 1345 bp, Artificial Sequence
  Description of Artificial Sequence/note = synthetic construct
tcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattcaagctt

SEQ ID NO: 69 - DNA, 684 bp, Artificial Sequence
  Description of Artificial Sequence/note = synthetic construct
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcgg

SEQ ID NO: 70 - DNA, 4550 bp, Artificial Sequence
  Description of Artificial Sequence/note = synthetic construct
ttaattcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaagggatctgtagggcgcagtagtccagggtttccttgatgatgtcatacttatcctgtcccttttttttccacagctcgcggttgaggacaaactcttcgcggtctttccagtggggatcgacggtatcgtagagtcgaggccgctctagaactagtggatccggaacccttaatataacttcgtataatgtatgctatacgaagttattaggtccctcgacctgcaggaattctaccgggtaggggaggcgcttttcccaaggcagtctggagcatgcgctttagcagccccgctggcacttggcgctacacaagtggcctctggcctcgcacacattccacatccaccggtagcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcgaaggtcctcccgaggcccggcattctcgcacgcttcaaaagcgcacgtctgccgcgctgttctcctcttcctcatctccgggcctttcgacctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatccgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagcttgcagatctgcgactctagaggatctgcgactctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgcgactctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgcgactctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccccatcaagctgatccggaacccttaatataacttcgtataatgtatgctatacgaagttattaggtccctcgacctgcagcccaagctagcttatcgataccgatggaaatctgcagaggcctccgcagtcacctaatcactctcctcctcttcctgttccattcagagacgatctgcgcacctgtacgatcactgaactgcacgctccgggactcacagcaaaaaagcttggtgatgtctggtccatatgaactgaaagctctccacctccagggacaggatatggagcaacaagtggtgttctccatgtcctttgtacaaggagaagaaagtaatgacaaaatacctgtggccttgggcctcaaggaaaagaatctgtacctgtcctgcgtgttgaaagatgataagcccactctacagctggagagtgtagatcccaaaaattacccaaagaagaagatggaaaagcgatttgtcttcaacaagatagaaatcaataacaagctggaatttgagtctgcccagttccccaactggtacatcagcacctctcaagcagaaaacatgcccgtcttcctgggagggaccaaaggcggccaggatataactgacttcaccatgcaatttgtgtcttcctaaagtcgaccacgttactggccgaagccgcttggaataaggccggtgtgcgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtacccc

SEQ ID NO: 71 - DNA, 22 bp, Artificial Sequence
  Description of Artificial Sequence/note = synthetic construct
tccaatttactgaccgtacacc

SEQ ID NO: 72 - DNA, 21 bp, Artificial Sequence
  Description of Artificial Sequence/note = synthetic construct
gcaacaccattttttctgacc

SEQ ID NO: 73 - DNA, 42 bp, Artificial Sequence
  Description of Artificial Sequence/note = synthetic construct
aatatcttaattaaatctctagatgcttcgcgatgtacgggc

SEQ ID NO: 74 - DNA, 38 bp, Artificial Sequence
  Description of Artificial Sequence/note = synthetic construct
tagtcatatatgatcttaattaaaagcttgggtctccc

SEQ ID NO: 75 - DNA, 38 bp, Artificial Sequence
  Description of Artificial Sequence/note = synthetic construct
attaattaatcgatgcggccgctctagattaattaata

SEQ ID NO: 76 - DNA, 38 bp, Artificial Sequence
  Description of Artificial Sequence/note = synthetic construct
taattaattagctacgccggcgagatctatttaattat